(19) United States
(12) Patent Application Publication
(10) Pub. No.: US 2023/0240954 A1
(43) Pub. Date: Aug. 3, 2023

(54) USE OF ANTHRAQUINONE DYES AND OF FLUORESCENT DYES FOR DYEING KERATIN FIBRES, DYEING PROCESS AND COMPOSITION

(71) Applicant: L'OREAL, Paris (FR)

(72) Inventors: Annabelle EUDELINE-LY, Aulnay-Sous-Bois (FR); Hervé DAVID, Aulnay-Sous-Bois (FR); Christian BLAISE, Aulnay-Sous-Bois (FR); Abel MESSAVUSSU, Aulnay-Sous-Bois (FR)

(21) Appl. No.: 16/468,860

(22) PCT Filed: Dec. 21, 2017
(86) PCT No.: PCT/EP2017/084192

§ 371 Date: Jun. 12, 2019

(30) Foreign Application Priority Data
Dec. 22, 2016 (FR) 1663123

(51) Int. Cl. A61K 8/35; A61K 8/46; A61Q 5/06; A61K 8/49

(52) U.S. Cl. CPC A61K 8/355; A61K 8/466; A61Q 5/065; A61K 8/4926; A61K 2800/434

(57) ABSTRACT

Use of anthraquinone dyes and of fluorescent dyes for dyeing keratin fibres, dyeing process and composition. The present invention relates to a process for dyeing keratin fibres, in particular human keratin fibres such as the hair, using a) one or more particular anthraquinone dyes and b) one or more fluorescent direct dyes; to a composition comprising a) and b). The present invention also relates to a multi-compartment device containing the compounds defined above.

The present invention relates to a process for dyeing keratin fibres, in particular human keratin fibres such as the hair, using a) one or more particular anthraquinone dyes and b) one or more fluorescent direct dyes; to a composition comprising a) and b). The present invention also relates to a multi-compartment device containing the compounds defined above.

Many people have sought for a long time to modify the colour of their hair and in particular to mask their grey hair.

It is especially known practice to dye keratin fibres, in particular human keratin fibres, with dye compositions containing oxidation dye precursors, which are generally known as oxidation bases. These oxidation bases are colourless or weakly coloured compounds, which, when combined with oxidizing products, may give rise to coloured compounds via a process of oxidative condensation.

The shades obtained with these oxidation bases may be modified by combining them with couplers or colour modifiers. The variety of molecules used as oxidation bases and couplers allows a wide range of colours to be obtained.

Another well-known method consists in obtaining semi-permanent dyeing by applying to the keratin fibres direct dyes, which are coloured and colouring molecules that have affinity for said fibres.

The direct dyes conventionally used are chosen from nitrobenzene, anthraquinone, nitropyridine, azo, xanthene, acridine, azine and triarylmethane direct dyes. The chemical species may be nonionic, anionic (acidic dyes) or cationic (basic dyes). The direct dyes may also be natural dyes.

Conventional direct dyeing processes consist in applying to keratin fibres dye compositions comprising direct dyes. After application, a leave-on time is observed so as to allow the dye molecules to penetrate by diffusion into the fibres. On conclusion of the process, the fibres are rinsed.

In contrast with oxidation dyeing, these direct dyeing processes have a tendency to better protect the integrity of the fibres. The resulting colourings are generally chromatic, but, however, are only semi-temporary. The nature of the interactions that bind the direct dyes to the keratin fibres and their desorption from the surface and/or the core of the fibre are responsible for their weak dyeing power.

Although a wide range of colours is currently accessible, it generally proves necessary to combine three dyes of complementary colours—trichromatic principle—in order to obtain a natural shade (see, for example, WO 95/15144 and WO 95/01772). This tripartite combination does not, however, show good persistence with respect to repeated shampooing. It generally, or even systematically, induces an unaesthetic changing of the colour, which the consumer finds dissuasive.

Another aim of the invention is thus to be able to dye light keratin fibres efficiently in chestnut-brown, dark chestnut-brown with a glint or even black, by mixing direct dyes, and preferably only two types of direct dye.

These colourings are furthermore not sufficiently fast in the face of external agents such as light or perspiration.

Thus, there is a real need to implement processes for the direct dyeing of keratin fibres, in particular of human keratin fibres such as the hair, which do not have the drawbacks mentioned above, i.e. which make it possible especially to lead to natural colourings that have good properties, especially in terms of chromaticity, power, intensity, sheen and selectivity, and which are persistent with respect to shampooing.

The Applicant has found, surprisingly, that a process for dyeing keratin fibres using a) one or more anthraquinone dyes chosen from those of formulae (I) and (II) and b) one or more fluorescent direct dyes makes it possible to achieve the objectives mentioned above; especially to lead to natural chestnut-brown, dark chestnut-brown with a glint or even black colourings, which are not only powerful and shiny, but also shampoo-resistant.

One subject of the present invention is especially a process for dyeing keratin fibres, in particular human keratin fibres such as the hair, comprising the application to said keratin fibres of ingredients a) and b) below:
(a) one or more anthraquinone dyes chosen from the compounds of formulae (I) and/or (II) below, the optical isomers thereof, the geometrical isomers thereof, the tautomers thereof, the solvates thereof, and mixtures thereof:

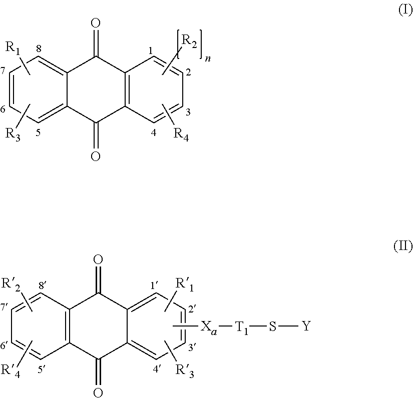

in which formula (I) or (II):
Xₐ, which may be identical or different, represents a bond, a heteroatom or a group chosen from an oxygen or sulfur atom, N(Rₐ), CO, SO, SO₂, or combinations thereof such as —O—CO—, —CO—O—, —NH—CO— or —CO—NH—, with Rₐ representing a hydrogen atom or a (C₁-C₆)alkyl group optionally substituted with one or more hydroxyl groups; preferably, Xₐ represent N(Rₐ) and particularly NH;
   Y represents: i) a hydrogen atom; ii) an alkali metal; iii) an alkaline-earth metal; iv) an ammonium group: N⁺R^{a}R^{b}R^{g}R^{d} or a phosphonium group: P⁺R^{a}R^{b}R^{g}R^{d} with R^{a}, R^{b}, R^{g} and R^{d}, which may be identical or different, representing a (C₁-C₄)alkyl group; or v) a thiol-function protecting group; or vi) the group (III) below:

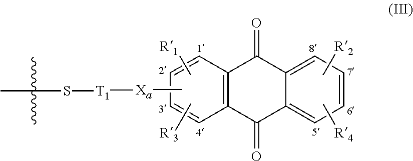

R₁, R₂, R₃, R₄, R′₁, R′₂, R′₃ and R′₄, which may be identical or different, represent an atom or a group chosen from:
   hydrogen;
   halogen such as bromine and chlorine,
   hydroxyl,
   C₁-C₄ alkoxy,
   hydroxysulfonyl (—SO₃H) or sulfonate (—SO₃⁻, M⁺), with M⁺ representing a cationic counterion, in particular an alkali metal, alkaline-earth metal or ammonium, such as Na⁺ or K⁺;
   optionally substituted C₁-C₆ alkyl,
   —NR₅R₆ in which R₅ and R₆, which may be identical or different, represent an atom or radical chosen from: i) hydrogen, ii) (C₁-C₄)alkylcarbonyl such as methylcarbonyl (—COCH₃), iii) arylsulfonyl such as phenylsulfonyl (—SO₂Ph), iv) Het-ALK-C(O)— with Het representing a heterocycloalkyl group which is optionally substituted, especially with one or more (C₁-C₄)alkyl groups and ALK represents a (C₁-C₆)alkylene group optionally substituted with one or more hydroxyl or (di)(hydroxy)(C₁-C₄)(alkyl)amino groups; Het-ALK-C(O)— is such as (piperidin-1-yl)acetyl, 2-methyl-2-(piperidin-1-yl)propanoyl, 2-(morpholin-4-yl)ethyl-β-alaninoyl, v) optionally substituted aryl, in particular phenyl optionally substituted with at least one radical chosen from a) C₁-C₆ alkyl, b) hydroxyl, c) hydroxysulfonyl, d) C₁-C₄ alkoxy, e) carboxyl (—COOH), f) (C₁-C₄)alkoxycarbonyl, g) amino, h) (di)(C₁-C₄)alkylamino, one of the alkyl radicals possibly being substituted with a hydroxyl or hydroxysulfonyl radical —SO₃H, or —OSO₃H, vi) optionally substituted aryl(C₁-C₄)alkyl, in particular benzyl optionally substituted with a (di)(C₁-C₄)(alkyl)amino group, viii) optionally substituted C₁-C₂₀ alkyl, optionally interrupted with one or more heteroatoms and/or with one or more groups comprising at least one heteroatom, preferably chosen from oxygen, nitrogen and sulfur, CO, SO, SO₂ or combinations thereof. when said alkyl radical is substituted, it is substituted with one or more atoms or groups chosen from: a) halogens, preferably one or more chlorine atoms, b) hydroxyl, c) (C₁-C₆)alkylcarbonylamino, in particular acylamino, d) 5- or 6-membered heterocycloalkyl such as tetrahydro-2H-pyran-4-amine, morpholino, piperidino or piperazino, e) (di)(C₁-C₄)(alkyl)amino, f) hydroxysulfonyl(C₁-C₄)alkylamino, or hydroxysulfonyloxy(C₁-C₄)alkylamino, g) (di)(hydroxy)(C₁-C₄)(alkyl)amino, h) 5- or 6-membered heteroaryl such as imidazole, and i) formylamino (—NHCOH);
   a group of formula (a) below:

—N(R₇)—X₁—W₁ (a)
in which formula (a):
R₇ represents a hydrogen or a C₁-C₄ alkyl radical,
X₁ represents a divalent radical chosen from C₁-C₂₀ alkylene optionally interrupted with one or more heteroatoms or groups chosen from oxygen, nitrogen and sulfur, CO, SO, SO₂, arylene such as phenylene, or combinations thereof; preferably, X₁ represents:
   (C₁-C₁₀)alkylene,
   —(C₁-C₁₀)alkylcarbonyl-,
   -carbonyl(C₁-C₁₀)alkyl-,
   —(C₁-C₁₀)alkylaminocarbonyl(C₁-C₁₀)alkyl-,
   —(C₁-C₁₀)alkylcarbonylamino(C₁-C₁₀)alkyl-,
   -phenyl(C₁-C₁₀)alkyl-,
   —(C₁-C₁₀)alkylphenyl-, or
   phenylene,
W₁ represents a cationic radical chosen from:

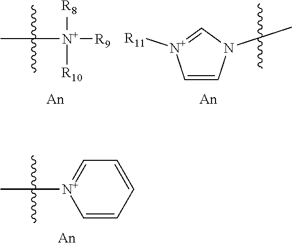

with R₈, R₉, R₁₀ and R₁₁, which may be identical or different, representing a C₁-C₆ alkyl group, a benzyl radical, a C₁-C₆ alkyl sulfonate radical; the radicals R₈ and R₉ may form, with the nitrogen atom to which they are attached, a saturated or unsaturated, optionally substituted 5- to 7-membered heterocycle, optionally comprising another non-nitrogen heteroatom, preferably an oxygen atom;
n is an integer ranging from 1 to 3; preferably, n is equal to 1 or 2, T₁ represents a linear or branched divalent hydrocarbon-based chain comprising from 1 to 20 carbon atoms, optionally interrupted with one or more heteroatoms or groups, or combinations thereof, chosen from oxygen, sulfur, N(R_{b}), C(O), —N⁺(R₈)(R₉)-An, optionally cationic and optionally substituted heteroaryl, such as imidazolium, An with R₈ and R₉, which may be identical or different, represent a C₁-C₆ alkyl radical; R_{b} representing a hydrogen atom or a (hydroxy)(C₁-C₄)alkyl group; preferably, said hydrocarbon-based chain is interrupted with one or more groups chosen from N(R_{b}), C(O), and a combination thereof such as —C(O)— N(R_{b})— or —N(R_{b})—C(O)—, and —N⁺(R₈)(R₉)-An, An is an organic or inorganic anionic counterion, which ensures the electrical neutrality of the dyes of formulae (I) and (II);
[symbol] being the part of the bond that is connected to the rest of the molecule; and
(b) one or more fluorescent dyes;
   it being understood that:
the anthraquinone dye(s) of formula (I) comprise at least one radical R₁, R₃ or R₄, other than a hydrogen atom, and the anthraquinone dye(s) of formula (II) comprise at least one radical R′₁, R′₂, R′₃ or R′₄ other than a hydrogen atom;
(a) the anthraquinone dye(s) of formula (I) or (II) and (b) the fluorescent dye(s) are applied to said keratin fibres together or sequentially; and
when the compounds of formula (I) or (II) are cationic and comprise a sulfonate group, then M⁺ and An may be absent to ensure the electrical neutrality of said molecule.

Another subject of the invention is a cosmetic composition comprising:
(a) one or more anthraquinone dyes chosen from the compounds of formulae (I) and (II) as defined previously; and
(b) one or more fluorescent dyes.

The combination of anthraquinone dye(s) of formula (I) or (II) and of fluorescent dye(s) makes it possible especially to obtain natural colourings that have good dyeing properties, especially in terms of chromaticity, power, intensity, sheen and selectivity.

Furthermore, the process and the composition according to the invention make it possible to dye light keratin materials efficiently in chestnut-brown, dark chestnut-brown with a glint or even black, by mixing direct dyes, and in particular only the ingredients a) and b) as defined previously as dyes, without the need to use additional (or complementary) dye(s) other than a) and b).

Moreover, the colourings obtained by means of the process and the composition according to the invention show good resistance to the various attacking factors to which the hair may be subjected, such as light, bad weather, washing and perspiration. They are in particular persistent with respect to shampooing, especially after at least three shampoo washes.

A subject of the present invention is also a multi-compartment device comprising a first compartment containing a composition comprising (a) one or more dyes of formula (I) or (II) as defined previously, and a second compartment containing a composition comprising (b) one or more fluorescent dyes as defined previously.

Another subject of the invention is the use of a) fluorescent dye(s) as defined previously, combined with b) one or more dyes of formula (I) or (II) as defined previously, for dyeing light keratin fibres, especially human keratin fibres such as the hair, in chestnut-brown, dark chestnut-brown or even black, without the need to use additional dye(s) other than a) or b).

Other subjects, characteristics, aspects and advantages of the invention will emerge even more clearly on reading the description and the examples that follow.

For the purposes of the present invention and unless otherwise indicated:
the “dyes” according to the invention absorb light in the visible range, i.e. at a wavelength λ_{abs} particularly between 400 and 700 nm inclusive;
a “thiol-protecting group” is a group known to those skilled in the art, for instance those described in the publications “Protective Groups in Organic Synthesis”, T. W. Greene, John Wiley & Sons ed., NY, 1981, pp. 193-217; “Protecting Groups”, P. Kocienski, Thieme, 3rd ed., 2005, chap. 5; and Ullmann's Encyclopedia, “Peptide Synthesis”, pp. 4-5, 2005 Wiley-VCH Verlag GmbH & Co. KGaA, Weinheim 10.1002/14356007.a19 157; preferably, the protected thiol function is such that Y represents a hydrogen atom; ii) an alkali metal; iii) an alkaline-earth metal; iv) an ammonium group: N⁺R^{a}R^{b}R^{g}R^{d} or a phosphonium group: P⁺R^{a}R^{b}R^{g}R^{d} with R^{a}, R^{b}, R^{g} and R^{d}, which may be identical or different, representing a hydrogen atom or a (C₁-C₄)alkyl group;
For the purposes of the present invention, the term “direct dye” means natural and/or synthetic dyes, which are soluble in the cosmetic medium, other than oxidation dyes; they are dyes which will diffuse superficially on the keratin fibres;
For the purposes of the present invention, the term “fluorescent dye” means a dye which is a coloured molecule (which absorbs visible light) and which itself imparts colour, and which, in contrast with a conventional dye, transforms the absorbed energy into light of a longer wavelength emitted in the visible part of the spectrum; in particular, the “fluorescent” dyes of the invention are capable of absorbing light in the visible range at a wavelength λ_{abs} of between 350 and 800 nm and of re-emitting in the visible range at a longer wavelength λₑₘ than that absorbed, of between 400 and 800 nm; the difference between the absorption and emission wavelengths, also known as the Stoke's shift, is between 1 nm and 100 nm. More preferentially, the fluorescent dyes of the invention are dyes that are capable of absorbing at a wavelength λ_{abs} of between 420 nm and 550 nm and of re-emitting in the visible range at a wavelength λₑₘ of between 470 and 600 nm;
the “fluorescent dyes” according to the present invention are to be differentiated from optical brighteners. Optical brighteners, also known as “brighteners” or “fluorescent brighteners” or “fluorescent brightening agents” or “fluorescent whitening agents or FWA” or “whiteners” or “fluorescent whiteners”, are colourless compounds, which do not impart a colour and are consequently not dyes since they do not absorb in the visible light range, but only absorb in the ultraviolet range (wavelength ranging from 200 to 400 nm) and transform the absorbed energy into fluorescent light of a longer wavelength emitted in the visible part of the spectrum in the blue range. The colour impression is then generated only by the purely fluorescent light that is predominantly blue (wavelength ranging from 400 to 500 nm).
the “blue-violet” dyes according to the invention are dyes which absorb light in the visible spectrum and which appear blue visually, i.e. which absorb light at an absorption wavelength λₘₐₓ greater than 550 nm and less than or equal to 700 nm, in particular λₘₐₓ between 560 nm and 700 nm, preferably in the blue range, i.e. λₘₐₓ between 580 and 650 nm;
the term “(hetero)aryl” means aryl and heteroaryl groups;
the “aryl” or “heteroaryl” radicals or the aryl or heteroaryl part of a radical may be substituted with at least one substituent borne by a carbon atom, chosen from:
   a C₁-C₆ and preferably C₁-C₄ alkyl radical optionally substituted with one or more radicals chosen from hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom;
   a hydroxyl or thiol group;
   a C₁-C₆ alkoxy or C₁-C₆ alkylthio radical;
   a (poly)hydroxy(C₂-C₆)alkoxy radical;
   an amino radical;
   a 5- or 6-membered heterocycloalkyl radical, preferentially morpholino, piperazino, piperidino or pyrolidino, which is optionally substituted with a (C₁-C₄) alkyl radical, preferentially methyl;
   a 5- or 6-membered heteroaryl radical, preferentially imidazolyl, optionally substituted with a (C₁-C₄)alkyl radical, preferentially methyl;
   an amino radical substituted with one or two identical or different C₁-C₆ alkyl radicals, optionally bearing at least:
      i) a hydroxyl group,
      ii) an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
      iii) a quaternary ammonium group —N⁺R′R″R′″, An⁻ for which R′, R″ and R′″, which may be identical or different, represent a C₁-C₄ alkyl group and An⁻ represents an anionic counterion,
      iv) or an optionally cationic 5- or 6-membered heteroaryl radical, preferentially imidazolium, optionally substituted with a (C₁-C₄)alkyl radical, preferentially methyl;
   an acylamino radical (—N(R)—C(O)—R′) in which the R radical is a hydrogen atom or a C₁-C₄ alkyl radical optionally bearing at least one hydroxyl group and the R′ radical is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)₂—N(R)—) in which the radical R represents a hydrogen atom or a C₁-C₄ alkyl radical optionally bearing at least one hydroxyl group and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical optionally bearing at least one hydroxyl group;
   a carboxylic radical in acid or salified (preferably with an alkali metal or a substituted or unsubstituted ammonium) form;
   a cyano group;
   a nitro or nitroso group;
   a polyhaloalkyl group, preferably trifluoromethyl;
the cyclic or heterocyclic part of a non-aromatic radical may be substituted with at least one substituent chosen from the following groups:
   hydroxyl;
   C₁-C₄ alkoxy, C₂-C₄ (poly)hydroxyalkoxy;
   C₁-C₄ alkyl;
   alkylcarbonylamino (R—C(O)—N(R′)—) in which the radical R′ is a hydrogen atom or a C₁-C₄ alkyl radical optionally bearing at least one hydroxyl group, and the radical R is a C₁-C₂ alkyl radical or an amino radical optionally substituted with one or two C₁-C₄ alkyl groups, which may be identical or different, themselves optionally bearing at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;
   alkylcarbonyloxy (R—C(O)—O—) in which the radical R is a C₁-C₄ alkyl radical or an amino group optionally substituted with one or two identical or different C₁-C₄ alkyl groups themselves optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
   alkoxycarbonyl (R—X—C(O)—) in which the radical R is a C₁-C₄ alkoxy radical, X is an oxygen atom or an amino group optionally substituted with a C₁-C₄ alkyl group itself optionally bearing at least one hydroxyl group, said alkyl radical possibly forming with the nitrogen atom to which it is attached a saturated or unsaturated, optionally substituted 5- to 7-membered heterocycle, optionally comprising at least one other nitrogen or non-nitrogen heteroatom;
a cyclic or heterocyclic radical, or a non-aromatic part of an aryl or heteroaryl radical, may also be substituted with one or more oxo groups;
an “aryl” radical represents a monocyclic or fused or non-fused polycyclic carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl;
a “cationic heteroaryl radical” is a heteroaryl group as defined above which comprises an endocyclic or exocyclic cationic group;
   when the charge is endocyclic, it is included in the electron delocalization via the mesomeric effect; for example, it is a pyridinium, imidazolium or indolinium group:

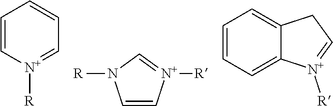

with R and R′ being a heteroaryl substituent as defined previously and particularly a (hydroxy)(C₁-C₈)alkyl group, such as methyl;
   when the charge is exocyclic, it is not included in the electron delocalization via the mesomeric effect; for example, it is an ammonium or phosphonium substituent R⁺, such as trimethylammonium, which is outside the heteroaryl, such as pyridyl, indolyl, imidazolyl or naphthalimidyl, in question:

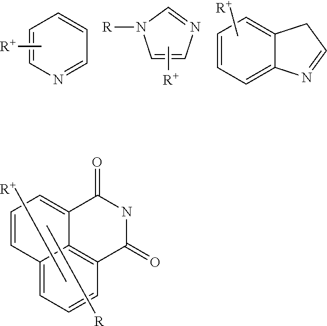

with R being a heteroaryl substituent as defined below and R⁺ an ammonium RₐR_{b}R_{c}N⁺—, phosphonium RₐR_{b}R_{c}P⁺— or ammonium RₐR_{b}R_{c}N⁺—(C₁-C₆)alkylamino, RₐR_{b}R_{c}N⁺—(C₁-C₆)alkyl or RₐR_{b}R_{c}N⁺—(C₁-C₆)alkoxy group with Rₐ, R_{b} and R_{c}, which may be identical or different, being a (C₁-C₈)alkyl group such as methyl;
a “heteroaryl radical” represents a 5- to 22-membered, monocyclic or fused or non-fused polycyclic group, comprising from 1 to 6 heteroatoms chosen from nitrogen, oxygen and sulfur, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridinyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthylyl and the ammonium salt thereof;
a “heterocyclic radical” is a radical which may contain one or two unsaturations, but is a monocyclic or fused or non-fused polycyclic, 5- to 22-membered non-aromatic radical comprising from 1 to 6 heteroatoms chosen from nitrogen, oxygen and sulfur;
a “heterocycloalkyl radical” is a heterocyclic radical comprising at least one saturated ring;
the term “divalent hydrocarbon-based radical” means a linear or branched, saturated or unsaturated chain, which may thus comprise one or more unsaturations, comprising from 1 to 20 carbon atoms, in particular between 2 and 14 carbon atoms, preferably between 2 and 6 carbon atoms;
an “alkyl” radical is a linear or branched C₁-C₁₀, in particular C₁-C₈, more particularly C₁-C₆ and preferably C₁-C₄ hydrocarbon-based radical;
the term “optionally substituted” applied to the alkyl radical implies that said alkyl radical may be substituted with one or more radicals chosen from the following radicals: i) hydroxyl, ii) C₁-C₄ alkoxy, iii) R—Z—C(X)—Y— with X, Y and Z representing an oxygen or sulfur atom or N(R′), or alternatively X and/or Z represent a bond, R and R′, which may be identical or different, represent a hydrogen atom or a (C₁-C₆)alkyl group, preferably, X represents an oxygen atom, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom; v) or a quaternary ammonium group N⁺R′R″R′″, An⁻ for which R′, R″ and R′″, which may be identical or different, represent a C₁-C₄ alkyl group, or alternatively —N⁺R′R″R′″ forms a heteroaryl such as imidazolium optionally substituted with a C₁-C₄ alkyl group and An⁻ represents the anionic counterion, vi) carboxyl C(O)H, vii) carboxylate C(O)O⁻, M⁺ with M⁺ representing a cationic counterion such as alkali metal or alkaline-earth metal, viii) sulfonic —SO₃H, ix) sulfonate —SO₃⁻, M⁺ with M⁺ as defined previously, and x) cyano;
an “alkoxy radical” is an alkyl-oxy radical for which the alkyl radical is a linear or branched C₁-C₈ and preferentially C₁-C₆ hydrocarbon-based radical;
when the alkoxy group is optionally substituted, this implies that the alkyl group is optionally substituted as defined above;
the term “organic or mineral acid salt” is more particularly intended to mean salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)—OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)—OH; xiv) triflic acid CF₃SO₃H; and xv) tetrafluoroboric acid HBF₄;
the term “anionic counterion” means an anion or an anionic group derived from an organic or mineral acid which counterbalances the cationic charge of the dye; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) carboxylates Alk-C(O)—OH with Alk representing a (C₁-C₆)alkyl group optionally substituted with one or more hydroxyl or carboxylate groups such as citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻, xiii) phosphates O═P(OH)₂—O⁻, O═P(O⁻)₂—OH O═P(O⁻)₃, HO—[P(O)(O⁻)]_{w}—P(O)(O⁻)₂ with w being an integer; xiv) acetate; xv) triflate; and xvi) borates such as tetrafluoroborate, and xvii) disulfate (O=)₂S(O⁻)₂ or SO₄²⁻ and monosulfate HSO₄⁻;
   the anionic counterion, derived from the organic or mineral acid salt, ensures the electrical neutrality of the molecule; thus, it is understood that when the anion comprises several anionic charges, then the same anion may serve for the electrical neutrality of several cationic groups in the same molecule or else may serve for the electrical neutrality of several molecules; for example, a dye which contains two cationic groups may contain either two “singly charged” anionic counterions or a “doubly charged” anionic counterion such as (O=)₂S(O⁻)₂ or O═P(O⁻)₂—OH;

In particular, the anionic counterions are chosen from halides such as chloride, bromide, fluoride or iodide; a hydroxide; a sulfate; a hydrogen sulfate; a linear or branched C₁-C₆ alkyl sulfate, such as the methylsulfate or ethylsulfate ion; carbonates and hydrogen carbonates; carboxylic acid salts such as formate, acetate, citrate, tartrate and oxalate; linear or branched C₁-C₆ alkylsulfonates, such as the methylsulfonate ion; arylsulfonates for which the aryl part, preferably phenyl, is optionally substituted with one or more C₁-C₄ alkyl radicals, for instance 4-tolylsulfonate; alkylsulfonates such as mesylate;
the term “chemical oxidizing agent” means any oxidizing agent other than atmospheric oxygen conventionally used in the field. Thus, mention may be made of hydrogen peroxide, urea peroxide, alkali metal bromates, persalts such as perborates and persulfates, and also enzymes, among which mention may be made of peroxidases, 2-electron oxidoreductases such as uricases, and 4-electron oxygenases such as laccases. Preferably, the chemical oxidizing agent is hydrogen peroxide.
Moreover, the addition salts that may be used in the context of the invention are in particular chosen from addition salts with a cosmetically acceptable base such as basifying agents as defined below, for instance alkali metal hydroxides, such as sodium hydroxide or potassium hydroxide, aqueous ammonia, amines or alkanolamines.
the expression “at least one” is equivalent to “one or more”;
the limits of a range of values are included in that range, in particular in the expressions “between” and “ranging from . . . to . . . ”; and
the expression “inclusive” for a range of values means that the limits of that range are included in the defined range.

(a) Anthraquinone Dyes

The process for dyeing keratin fibres according to the present invention comprises the application to said keratin fibres of (a) one or more anthraquinone dyes chosen from the compounds of formulae (I) and (II) as defined previously, the optical isomers thereof, the geometrical isomers thereof, the tautomers thereof, the solvates thereof, and mixtures thereof.

In particular, the dyes of formulae (I) and (II) are violet, blue or green.

According to a particular embodiment of the invention, a) the anthraquinone dye(s) of the invention are chosen from the dyes of formula (I) as defined previously.

According to a particular embodiment of the invention, a) the anthraquinone dye(s) of the invention are chosen from one or more dyes of formula (I) and n is equal to 0. According to another embodiment, n is equal to 1. Preferably, the substituents R₁, R₂, R₃ and R₄ are in positions 1, 4, 5 and 8.

According to a particular embodiment of the invention, the dyes are of formula (I), with R₁ and R₃ representing an atom or group chosen from i) hydroxyl, ii) arylamino or aryl(C₁-C₆)alkylamino with the aryl group representing an optionally substituted aryl group, in particular a phenyl group optionally substituted with one or more groups chosen from hydroxyl, (C₁-C₄)alkyl, carboxyl, (C₁-C₄)alkoxycarbonyl, (di)(hydroxy)(C₁-C₄)(alkyl)amino, tri(C₁-C₄)alkylammonium, (C₁-C₄)alkoxy, (di)hydroxysulfonyloxy(C₁-C₄)alkylamino, hydroxysulfonyl, iii) (di)(hydroxy)(C₁-C₆)(alkyl)amino, iv) (di)(C₁-C₄)(alkyl)amino(C₁-C₆)alkylamino, v) (C₁-C₄)alkylcarbonylamino(C₁-C₆)alkylamino, vi) arylsulfonylamino with the aryl group possibly being optionally substituted, aryl preferably representing a phenyl group, vii) (di)halo(C₁-C₄)alkylamino, viii) (di)(hydroxy)(C₁-C₄)alkoxy(C₁-C₄)alkylamino, ix) (di)tri(C₁-C₄)alkylammonium(C₁-C₆)alkylamino, x) heterocycloalkyl(C₁-C₆)alkylamino, heterocycloalkyl(C₁-C₄)alkylcarbonylamino, heterocycloalkyl(C₁-C₄)alkylamino(C₁-C₄)alkylcarbonylamino or heterocycloalkyl(C₁-C₄)alkylcarbonylamino(C₁-C₆)alkylamino, said heterocycloalkyl possibly being optionally cationic and/or substituted especially with one or more (C₁-C₄)alkyl or benzyl groups, xi) halo(C₁-C₄)alkylcarbonylamino(C₁-C₆)alkylamino, xii) hydroxysulfonyl, xiii) halo such as chlorine, xiv) heteroaryl(C₁-C₆)alkylamino, said heteroaryl possibly being optionally cationic and/or substituted especially with one or more (C₁-C₄)alkyl groups, xv) heteroaryl(C₁-C₄)alkylcarbonylamino(C₁-C₆)alkylamino, said heteroaryl possibly being optionally cationic and/or substituted especially with one or more (C₁-C₄)alkyl groups, xvi) R′R″N— with R′ and R″, which may be identical or different, representing a hydrogen, a (halo)(C₁-C₄)alkylcarbonylamino(C₁-C₆)alkyl group, a (halo)(C₁-C₄)alkylaminocarbonyl (C₁-C₆)alkyl group, (halo)(C₁-C₄)alkylcarbonyl group, a carboxy(C₁-C₆)alkyl group, the alkyl group possibly being substituted with one or more amino or hydroxyl groups, a (poly)hydroxy(C₁-C₆)alkyl group, xvii) (C₁-C₁₆)alkylaminocarbonylamino(C₁-C₆)alkylamino, xviii) formylamino(C₁-C₆)alkylamino, xix) (hydroxy)(C₁-C₆)alkylamino(C₁-C₆)alkylamino, xix) (poly)hydroxysulfonyl(C₁-C₆)alkylamino(C₁-C₆)alkylamino, xx) sulfonato(C₁-C₆)alkyl(di(C₁-C₄)alkyl)ammonium(C₁-C₆)alkylamino, xxi) hydroxysulfonyl(C₁-C₆)alkoxy(C₁-C₆)alkylamino, xxi) heteroaryl(C₁-C₄)alkylcarbonylamino, said heteroaryl possibly being optionally cationic and/or substituted especially with one or more (C₁-C₄)alkyl groups, xxii) heterocycloalkyl(C₁-C₄)alkylcarbonylamino, said heterocycloalkyl possibly being optionally cationic and/or substituted especially with one or more (C₁-C₄)alkyl or benzyl groups, xxiii) (di)(C₁-C₄)(alkyl)amino(C₁-C₆)alkylamino, the (C₁-C₆)alkyl group possibly being substituted with one or more hydroxyl groups, xxiv) heterocycloalkylamino(C₁-C₆)alkylamino or heterocycloalkylamino, said heterocycloalkyl possibly being optionally cationic and/or substituted especially with one or more (C₁-C₄)alkyl or benzyl groups, xxv) heteroarylalkylamino(C₁-C₆)alkylamino or heteroarylalkylamino, said heteroaryl possibly being optionally cationic and/or substituted especially with one or more (C₁-C₄)alkyl groups, xxvi) tri(C₁-C₆)alkylammonium(C₁-C₆)alkylamino, xxvii) (C₁-C₄)alkoxycarbonyl(C₁-C₆)alkyl(di(C₁-C₄)alkyl)ammonium(C₁-C₆)alkylamino, xxviii) carboxylato(C₁-C₆)alkyl(di(C₁-C₄)alkyl)ammonium(C₁-C₆)alkylamino, xxix) carboxy(C₁-C₆)alkylamino(C₁-C₆)alkylamino, xxx) aryl(C₁-C₄)alkyl(di(C₁-C₄)alkyl)ammonium(C₁-C₆)alkylamino, xxxi) sulfonic SO₃H or sulfonate SO₃—, M⁺ with M⁺ representing a cationic counterion, xxxii) (C₁-C₆)alkyl, xxxiii) hydroxysulfonyl(C₁-C₄)amino, xxxiv) phenylsulfonylamino.

Preferably, the dyes of formula (I) are such that n is equal to 1 and the groups R₂ and R₄ are in positions 1 and 4.

According to a particular embodiment of the invention, the dyes are of formula (I) with R₂ and R₄ representing a hydrogen atom.

According to a particular embodiment of the invention, the dyes are of formula (I) with R₂ and R₄, which may be identical or different, representing a hydrogen atom or a group as defined previously for R₁ and R₃ i) to xxxii), preferably chosen from:
hydroxyl,
(di)(C₁-C₄)(alkyl)amino,
(C₁-C₄)alkoxy, heterocycloalkyl(C₁-C₆)alkylamino, said heterocycloalkyl possibly being optionally cationic and/or substituted especially with one or more (C₁-C₄)alkyl or benzyl groups,
arylamino or aryl(C₁-C₆)alkylamino with the aryl group representing an optionally substituted aryl group, in particular a phenyl group optionally substituted with one or more groups chosen from (C₁-C₄)alkyl, (di)(hydroxy)(C₁-C₄)(alkyl)amino, (C₁-C₄)alkoxy and tri(C₁-C₄)alkylammonium,
aryl(C₁-C₄)alkyl(di(C₁-C₄)alkyl)ammonium(C₁-C₆)alkylamino,
(di)(C₁-C₄)(alkyl)amino(C₁-C₆)alkylamino, and
hydroxy(C₁-C₄)(alkyl)amino(C₁-C₆)alkylamino.

According to another particular embodiment of the invention, the dyes are of formula (I), with R₂ and R₃ representing a hydrogen atom, and R₁ and R₄ are preferably in positions 8 and 4, respectively, and R₁ and R₄ are as defined previously, in particular represent an atom or group as defined previously for R₁ and R₃ i) to xxxii), preferably R₁ and R₄ are chosen from:
halogen such as chlorine,
(di)(C₁-C₄)(alkyl)amino,
(C₁-C₄)alkylcarbonylamino,
heterocycloalkyl(C₁-C₆)alkylamino, heterocycloalkyl(C₁-C₄)alkylcarbonylamino or heterocycloalkyl(C₁-C₄)alkylamino(C₁-C₄)alkylcarbonylamino, said heterocycloalkyl may be optionally cationic and/or substituted especially with one or more (C₁-C₄)alkyl or benzyl groups,
tri(C₁-C₄)alkylammonium(C₁-C₆)alkylamino,
arylamino or aryl(C₁-C₆)alkylamino with the aryl group representing an optionally substituted aryl group, in particular a phenyl group optionally substituted with one or more groups chosen from (C₁-C₄)alkyl, (di)(hydroxy)(C₁-C₄)(alkyl)amino, (C₁-C₄)alkoxy and tri(C₁-C₄)alkylammonium,
(di)(C₁-C₄)(alkyl)amino(C₁-C₆)alkylamino, and
(C₁-C₄)alkylcarbonylamino(C₁-C₆)alkylamino.

According to another particular embodiment of the invention, the dyes are of formula (I), with R₃ and R₄ representing a hydrogen atom, and R₁ and R₂ are preferably in positions 8 and 1, respectively, and R₁ and R₂, are as defined previously, in particular represent an atom or group as defined previously for R₁ and R₃ i) to xxxii), preferably R₁ and R₂ are chosen from:
(di)(hydroxy)(C₁-C₄)(alkyl)amino,
(di)(hydroxy)(C₁-C₄)(alkyl)amino(C₁-C₆)alkylamino,
tri(C₁-C₆)alkylammonium(C₁-C₆)alkylammonium,
tri(C₁-C₆)alkylammonium(C₁-C₆)alkylamino,
heterocycloalkyl(C₁-C₆)alkylamino, said heterocycloalkyl possibly being optionally cationic and/or substituted especially with one or more (C₁-C₄) alkyl or benzyl groups and
arylamino or aryl(C₁-C₆)alkylamino with the aryl group representing an optionally substituted aryl group, in particular a phenyl group optionally substituted with one or more groups chosen from (C₁-C₄)alkyl, (di)(hydroxy)(C₁-C₄)(alkyl)amino, (C₁-C₄)alkoxy, tri(C₁-C₄)alkylammonium.

According to another particular embodiment of the invention, the dyes are of formula (I), with R₁ and R₃ representing a hydrogen atom, and n is equal to 2, R₂ and R₄ are preferably in positions 1, 2 and 4, respectively, and R₂ and R₄, are as defined previously, in particular represent an atom or group as defined previously for R₁ and R₃ i) to xxxii), preferably R₂ and R₄ are chosen from:
hydroxyl,
(C₁-C₆)alkyl,
(di)(hydroxy)(C₁-C₄)(alkyl)amino, said (C₁-C₄)alkyl possibly being optionally substituted with a hydroxyl,
SO₃H or SO₃-M⁺,
(di)(hydroxy)(C₁-C₄)(alkyl)amino(C₁-C₆)alkylamino,
tri(C₁-C₄)alkylammonium(C₁-C₆)alkylamino,
halo(C₁-C₄)alkylcarbonylamino(C₁-C₄)alkylamino,
heterocycloalkyl(C₁-C₆)alkylamino, said heterocycloalkyl possibly being optionally cationic and/or substituted especially with one or more (C₁-C₄)alkyl or benzyl groups, and
arylamino or aryl(C₁-C₆)alkylamino with the aryl group representing an optionally substituted aryl group, in particular a phenyl group optionally substituted with one or more groups chosen from (C₁-C₄)alkyl, (di)(hydroxy)(C₁-C₄)(alkyl)amino, (C₁-C₄)alkoxy, tri(C₁-C₄)alkylammonium.

According to another particular embodiment of the invention, the dyes are of formula (I), n is equal to 0, R₁, R₃ and R₄ are preferably in positions 8, 5 and 4, respectively, and are as defined previously, in particular represent an atom or group as defined previously for R₁ and R₃ i) to xxxii), R₁ and R₃ preferably representing a hydrogen atom.

According to one embodiment, n is equal to 0, R₃ and R₄ represent a hydrogen atom, and R₁ is as defined previously and is in position 7 or 8.

According to another particular embodiment of the invention, a) the anthraquinone dye(s) are chosen from the symmetrical dyes bearing a disulfide group of formula (II′):

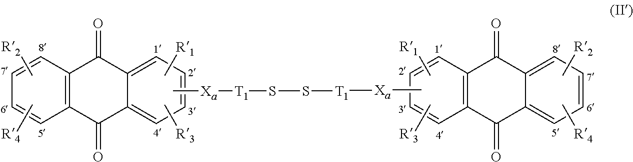

with R′₁, R′₂, R′₃ and R′₄, T₁ and Xₐ as defined previously, Xₐ, preferably represents a N(Rₐ) and particularly NH. More particularly, R′₂ and R′₄ represent a hydrogen atom and R′₁ and R′₃ are in particular as defined previously for R₁ and R₃ i) to xxxii); preferably, R′₃ and R′₄, which may be identical or different, represent a group chosen from (C₁-C₆)alkyl and (di)(hydroxy)(C₁-C₄)(alkyl)amino.

Preferably, T₁ represents a saturated linear divalent hydrocarbon-based chain comprising from 1 to 20 carbon atoms, preferably between 2 and 10 carbon atoms, optionally interrupted with one or more groups chosen from N(R_{b}), C(O), —N⁺(R₈)(R₉)-An, cationic heteroaryl such as imidazolium, An or combinations thereof, with Rₐ and R₉, which may be identical or different, represent a C₁-C₆ alkyl radical; R_{b} representing a hydrogen atom or a (C₁-C₄)alkyl group, preferably said hydrocarbon-based chain is interrupted with one or more groups chosen from —N⁺(R₈)(R₉)-An, N(R_{b}), C(O), and combinations thereof such as —C(O)—N(R_{b})— or —N(R_{b})—C(O)—, N⁺(R₈)(R₉)-An.

Preferably, T₁ represents a divalent group —(CH₂)ₙ-Tₐ-(CH₂)ₘ-T_{b}-(CH₂)ₚ— with Tₐ, and T_{b}, which may be identical or different, represent a bond or a group chosen from N(R_{b}), C(O), —N⁺(R₈)(R₉)-An, cationic heteroaryl such as imidazolium, An or combinations thereof, with R₈ and R₉, which may be identical or different, represent a C₁-C₆ alkyl radical; R_{b} representing a hydrogen atom or a (C₁-C₄)alkyl group, preferably —N⁺(R₈)(R₉)-An, —C(O)—N(R_{b})— or —N(R_{b})—C(O)—, n, m and p, which may be identical or different, represent an integer between 1 and 10 inclusive, with the sum n+m+p inclusively between 1 and 20, preferably between 2 and 10.

Preferentially, the groups R′₁ and R′₃ are in positions 2′ and 4′, and R′₂ and R′₄ are in positions 5′ and 8′.

More particularly, a) the anthraquinone dye(s) of the invention are chosen from the symmetrical dyes bearing a disulfide group of formula (II″):

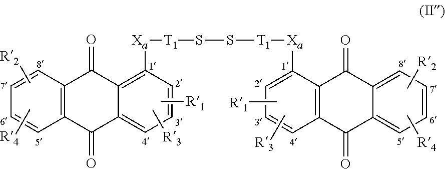

in which formula (II″) R′₁, R′₂, R′₃ and R′₄, T₁ and Xₐ are as defined previously for (II′).

According to a particular embodiment of the invention, a) the anthraquinone dye(s) of the invention are chosen from the dyes of formula (II) as defined previously in which Y represents a hydrogen atom or a thiol-function protecting group.

The anthraquinone dyes of the invention which contain a function SY of formula (II) may be in the covalent form —S—Y or the ionic form —S⁻Y⁺ depending on the nature of Y and the pH of the medium.

One particular embodiment concerns the anthraquinone thiol dyes of formula (II) bearing a function SY in which Y represents a hydrogen atom or an alkali metal. Advantageously, Y represents a hydrogen atom.

In accordance with another particular embodiment of the invention, in the abovementioned formula (II), Y is a protecting group known to those skilled in the art, for instance those described in the publications “Protective Groups in Organic Synthesis”, T. W. Greene, published by John Wiley & Sons, N Y, 1981, pages 193-217; “Protecting Groups”, P. Kocienski, Thieme, 3rd edition, 2005, chapter 5.

In particular, when Y represents a thiol-function protecting group, Y is chosen from the following radicals:
(C₁-C₄)alkylcarbonyl,
(C₁-C₄)alkylthiocarbonyl,
(C₁-C₄)alkoxycarbonyl,
(C₁-C₄)alkoxythiocarbonyl,
(C₁-C₄)alkylthiothiocarbonyl,
(di)(C₁-C₄)(alkyl)aminocarbonyl,
(di)(C₁-C₄)(alkyl)aminothiocarbonyl,
arylcarbonyl such as phenylcarbonyl,
aryloxycarbonyl,
aryl(C₁-C₄)alkoxycarbonyl,
(di)(C₁-C₄)(alkyl)aminocarbonyl such as dimethylaminocarbonyl;
(C₁-C₄)(alkyl)arylaminocarbonyl
carboxyl;
SO₃⁻; M⁺ with M⁺ representing an alkali metal such as sodium or potassium or alternatively M⁺ and M′ of formula (II) are absent;
optionally substituted aryl such as phenyl, dibenzosuberyl or 1,3,5-cycloheptatrienyl,
optionally substituted heteroaryl;
optionally cationic, optionally substituted heterocycloalkyl, the heterocycloalkyl group in particular represents a saturated or partially saturated 5-, 6- or 7-membered monocyclic group comprising from 1 to 4 heteroatoms chosen from oxygen, sulfur and nitrogen, such as di/tetrahydrofuranyl, di/tetrahydrothiophenyl, di/tetrahydropyrrolyl, di/tetrahydropyranyl, di/tetra/hexahydrothiopyranyl, dihydropyridyl, piperazinyl, piperidinyl, tetramethylpiperidyl, morpholinyl, di/tetra/hexahydroazepinyl, di/tetrahydropyrimidinyl, these groups being optionally substituted with one or more groups such as (C₁-C₄)alkyl, oxo or thioxo; or the heterocycle represents the following group:

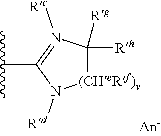

in which R′^{c}, R′^{d}, R′^{e}, R′^{f}, R′^{g} and R′^{h}, which may be identical or different, represent a hydrogen atom or a (C₁-C₄)alkyl group, or alternatively two groups R′^{g} with R′^{h}, and/or R′^{e} with R′^{f} form an oxo or thioxo group, or alternatively R′^{g} with R′^{e} together form a cycloalkyl; and v represents an integer between 1 and 3 inclusive; preferentially, R′^{c} to R′^{h} represent a hydrogen atom; and An represents a counterion;
isothiouronium —C(NR′^{c}R′^{d})═N⁺R′^{e}R′^{f}; An⁻ with R′^{c}, R′^{d}, R′^{e} and R′^{f}, which may be identical or different, represent a hydrogen atom or a (C₁-C₄)alkyl group; preferentially, R′^{c} to R′^{f} represent a hydrogen atom; and An⁻ represents a counterion;
isothiourea —C(NR′^{c}R′^{d})═NR′^{e}; with R′^{c}, R′^{d} and R′^{e} as defined previously;
optionally substituted (di)aryl(C₁-C₄)alkyl such as 9-anthracenylmethyl, phenylmethyl or diphenylmethyl optionally substituted with one or more groups in particular chosen from (C₁-C₄) alkyl, (C₁-C₄) alkoxy such as methoxy, hydroxyl, alkylcarbonyl or (di)(C₁-C₄)(alkyl)amino such as dimethylamino;
optionally substituted (di)heteroaryl(C₁-C₄)alkyl, the heteroaryl group in particular being a cationic or non-cationic, 5- or 6-membered monocyclic radical comprising from 1 to 4 heteroatoms chosen from nitrogen, oxygen and sulfur, such as pyrrolyl, furanyl, thiophenyl, pyridyl, pyridyl N-oxide groups such as 4-pyridyl or 2-pyridyl N-oxide, pyrylium, pyridinium or triazinyl, optionally substituted with one or more groups such as alkyl, particularly methyl; advantageously, the (di)heteroaryl(C₁-C₄)alkyl is (di)heteroarylmethyl or (di)heteroarylethyl;
CR¹R²R³ with R¹, R² and R³, which may be identical or different, representing a halogen atom or a group chosen from:
   (C₁-C₄)alkyl;
   (C₁-C₄)alkoxy;
   optionally substituted aryl such as phenyl optionally substituted with one or more groups, for instance (C₁-C₄)alkyl, (C₁-C₄)alkoxy or hydroxyl;
   optionally substituted heteroaryl such as thiophenyl, furanyl, pyrrolyl, pyranyl or pyridyl, optionally substituted with a (C₁-C₄)alkyl group;
   P(Z¹)R′¹R′²R′³ with R′¹ and R′², which may be identical or different, represent a hydroxyl, (C₁-C₄)alkoxy or alkyl group, R′³ represents a hydroxyl or (C₁-C₄)alkoxy group, and Z¹ represents an oxygen or sulfur atom;
sterically hindered ring such as the adamantyl group; and
optionally substituted alkoxy(C₁-C₄)alkyl such as methoxymethyl (MOM), ethoxyethyl (EOM) and isobutoxymethyl.

According to a particular embodiment, the thiol-protected anthraquinone dyes of formula (II) comprise a group Y chosen from i) aromatic cationic 5- or 6-membered monocyclic heteroaryl comprising from 1 to 4 heteroatoms chosen from oxygen, sulfur and nitrogen, such as oxazolium, isoxazolium, thiazolium, isothiazolium, 1,2,4-triazolium, 1,2,3-triazolium, 1,2,4-oxazolium, 1,2,4-thiadiazolium, pyrylium, pyridinium, pyrimidinium, pyrazinyl, pyrazinium, pyridazinium, triazinium, tetrazinium, oxazepinium, thiepinyl, thiepinium, imidazolium; ii) cationic 8- to 11-membered bicyclic heteroaryl such as indolinium, benzimidazolium, benzoxazolium, benzothiazolium, these monocyclic or bicyclic heteroaryl groups optionally being substituted with one or more groups such as alkyl, for instance methyl, or polyhalo(C₁-C₄)alkyl such as trifluoromethyl; iii) or the following heterocyclic:

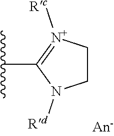

in which R′^{c} and R′^{d}, which may be identical or different, represent a hydrogen atom or a group (C₁-C₄)alkyl; preferentially, R′^{c} to R′^{d} represent a group (C₁-C₄)alkyl such as methyl; and An represents a counterion.

In particular, Y represents an alkali metal or a group chosen from oxazolium, isoxazolium, thiazolium, isothiazolium, 1,2,4-triazolium, 1,2,3-triazolium, 1,2,4-oxazolium, 1,2,4-thiadiazolium, pyrylium, pyridinium, pyrimidinium, pyrazinium, pyridazinium, triazinium and imidazolium, benzimidazolium, benzoxazolium, benzothiazolium, these groups being optionally substituted with one or more (C₁-C₄)alkyl groups, especially methyl.

In particular, Y represents an alkali metal or a protecting group such as:
(C₁-C₄)alkylcarbonyl, for instance methylcarbonyl or ethylcarbonyl;
arylcarbonyl such as phenylcarbonyl;
(C₁-C₄)alkoxycarbonyl;
aryloxycarbonyl,
aryl(C₁-C₄)alkoxycarbonyl,
(di)(C₁-C₄)(alkyl)aminocarbonyl such as dimethylaminocarbonyl;
(C₁-C₄)(alkyl)arylaminocarbonyl;
optionally substituted aryl such as phenyl;
5- or 6-membered monocyclic heteroaryl such as imidazolyl or pyridyl;
cationic 5- or 6-membered monocyclic heteroaryl such as pyrylium, pyridinium, pyrimidinium, pyrazinium, pyridazinium, triazinium or imidazolium; these groups being optionally substituted with one or more identical or different (C₁-C₄)alkyl groups such as methyl;
cationic 8- to 11-membered bicyclic heteroaryl such as benzimidazolium or benzoxazolium; these groups being optionally substituted with one or more identical or different (C₁-C₄)alkyl groups such as methyl;
cationic heterocycle having the following formula:

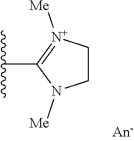 [SMILES: *C1=[N+](C)CCN1C]

isothiouronium —C(NH₂)═N⁺H₂; An⁻;
isothiourea —C(NH₂)═NH;
SO₃⁻; M⁺ with M⁺ representing an alkali metal such as sodium or potassium or alternatively M⁺ and M′ of formula (II) are absent.

Preferably, the anthraquinone dye(s) are chosen from the following compounds, the optical isomers thereof, the geometrical isomers thereof, the tautomers thereof, the solvates thereof, and mixtures thereof:

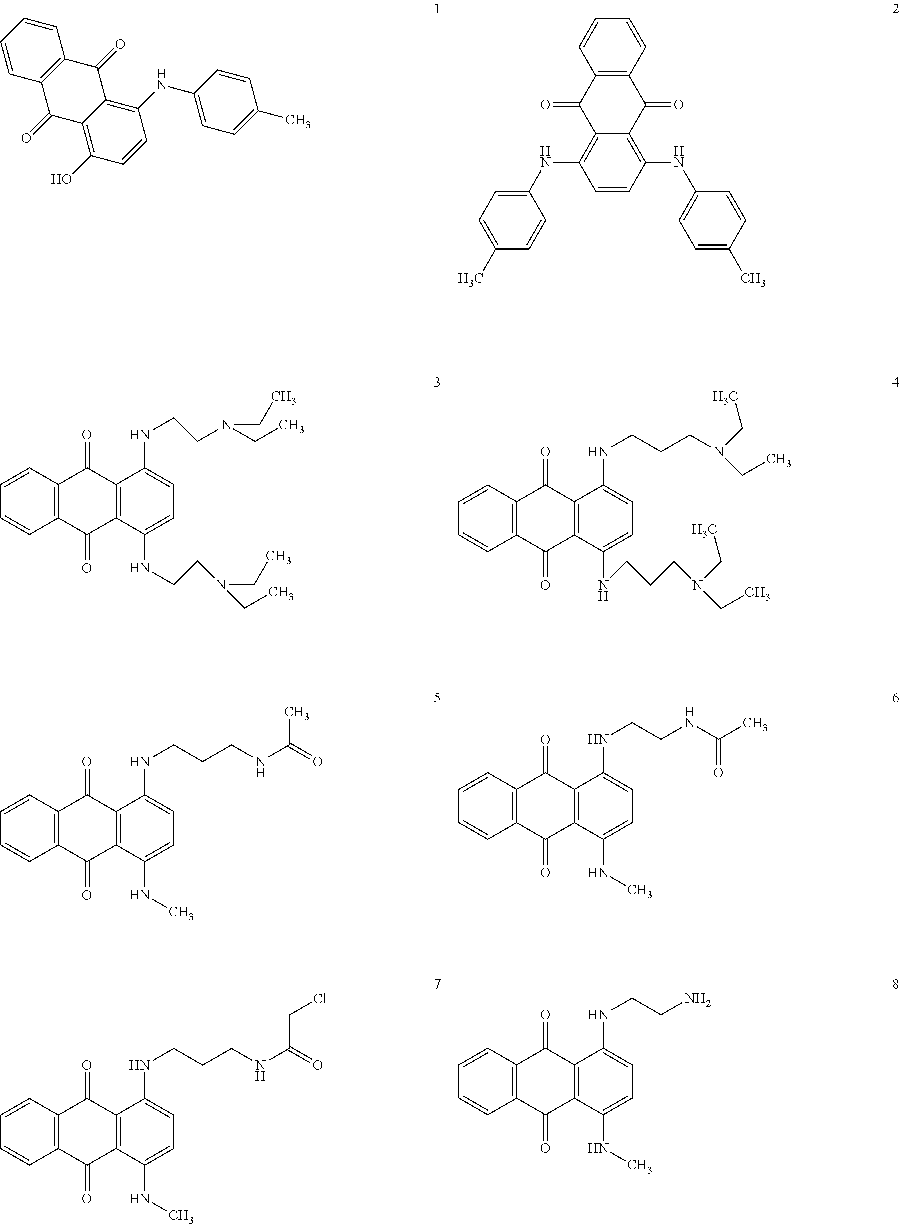

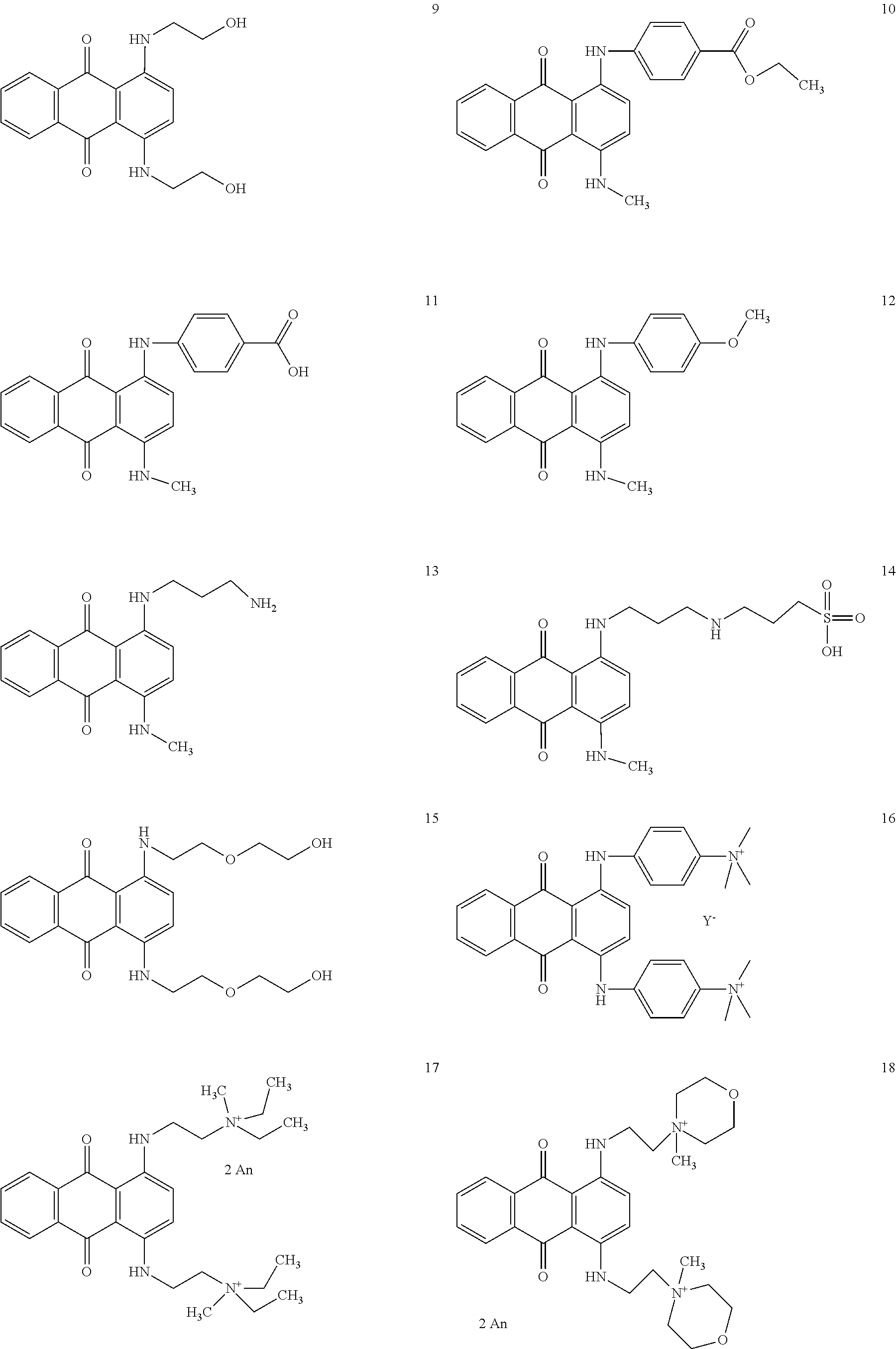

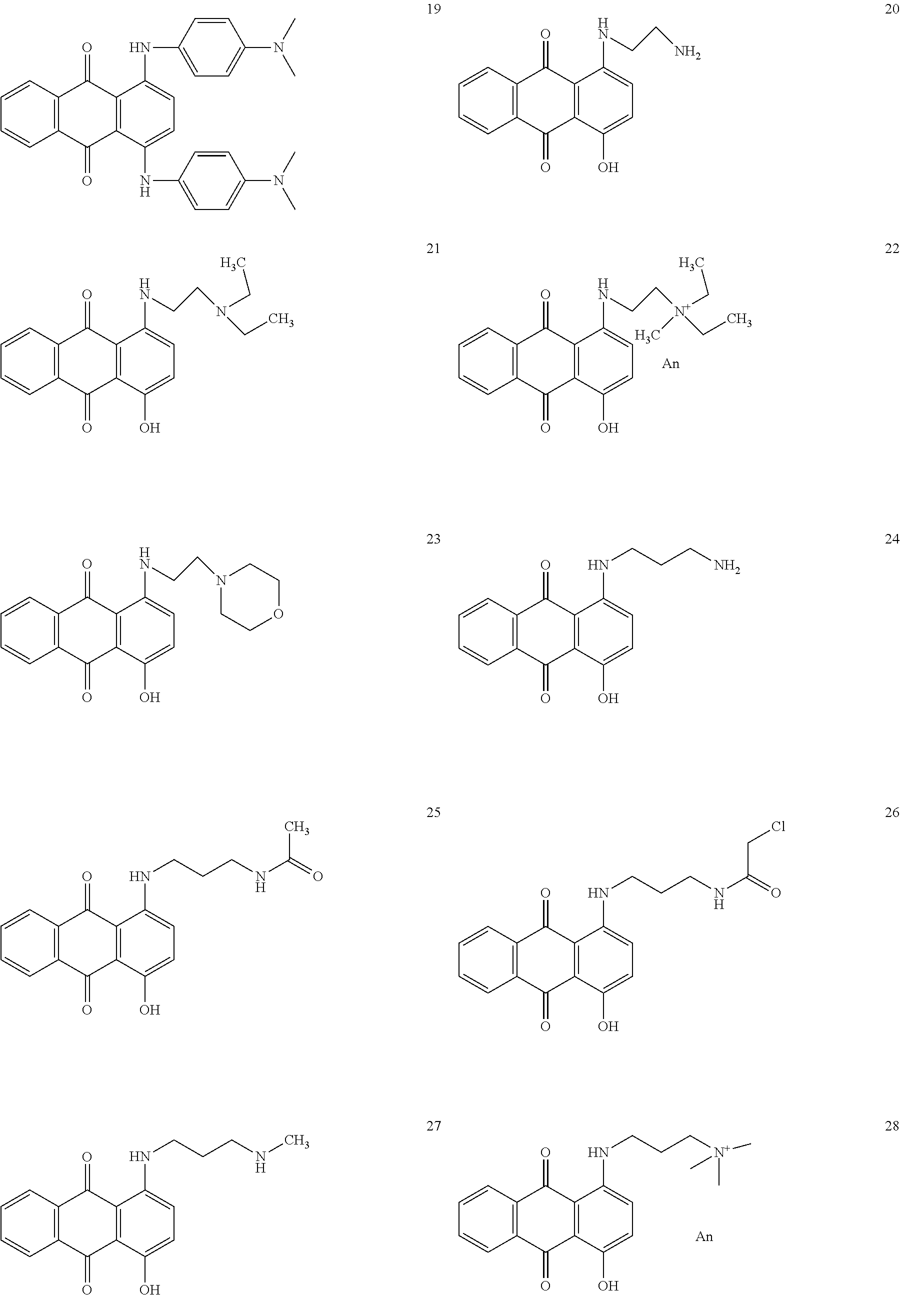

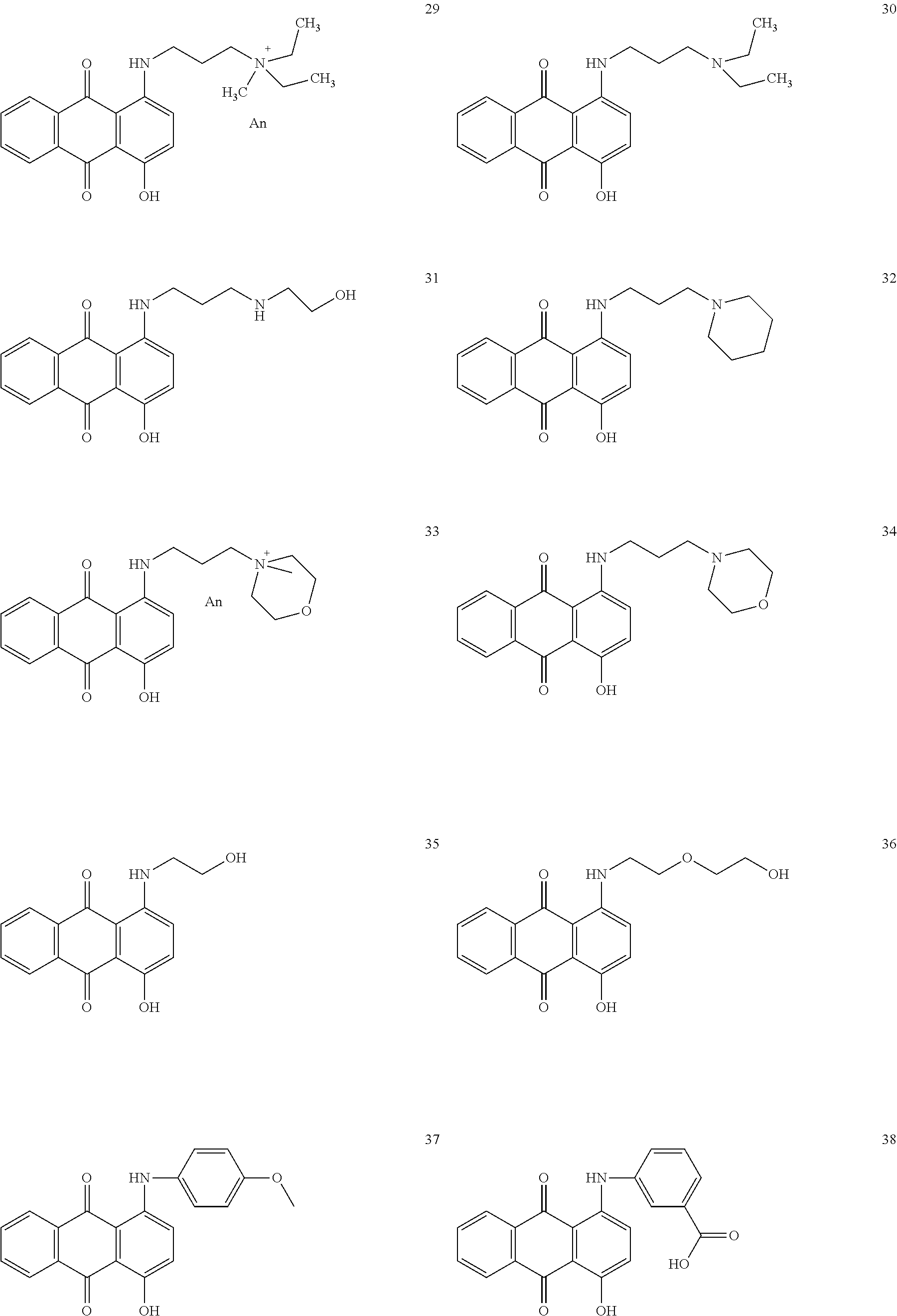

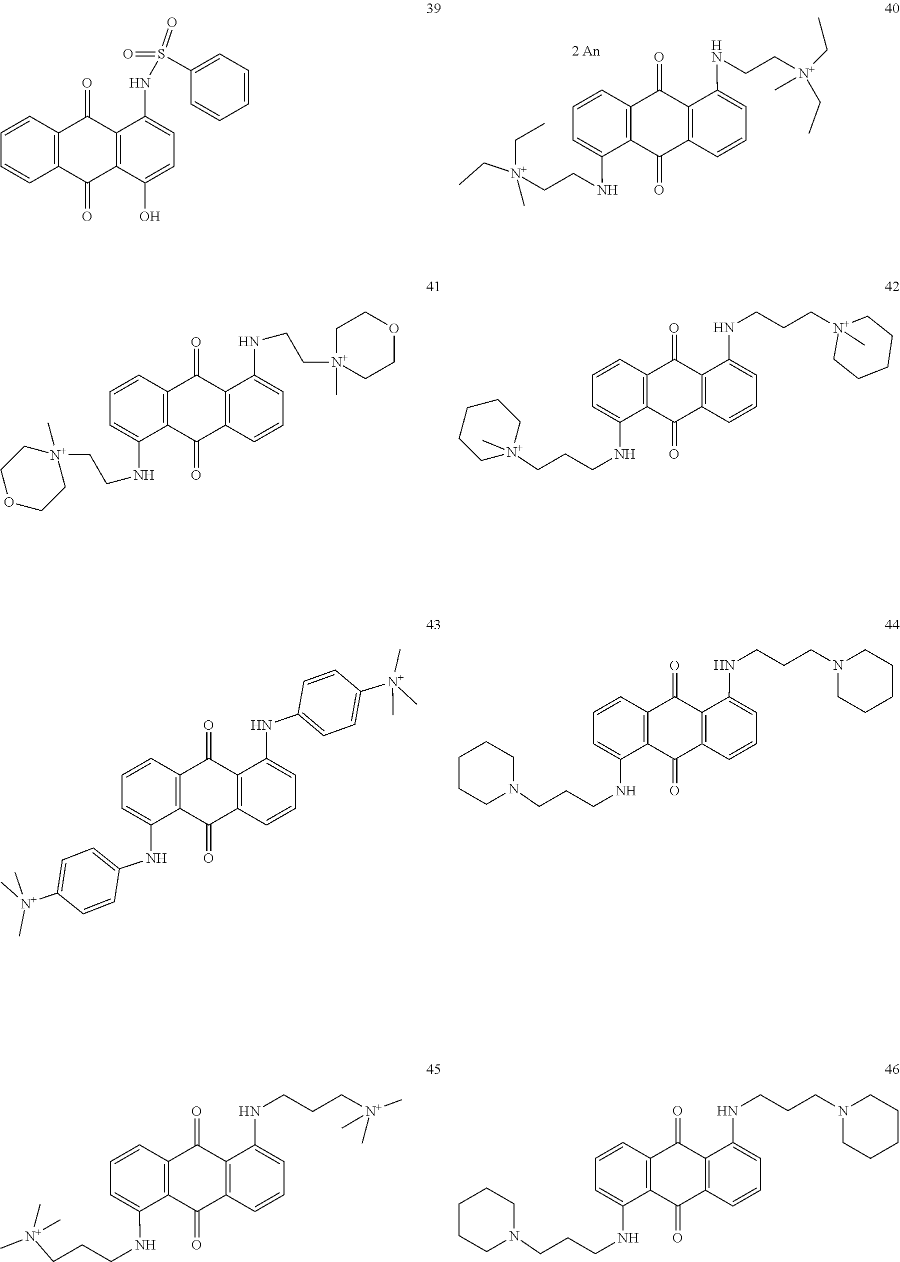

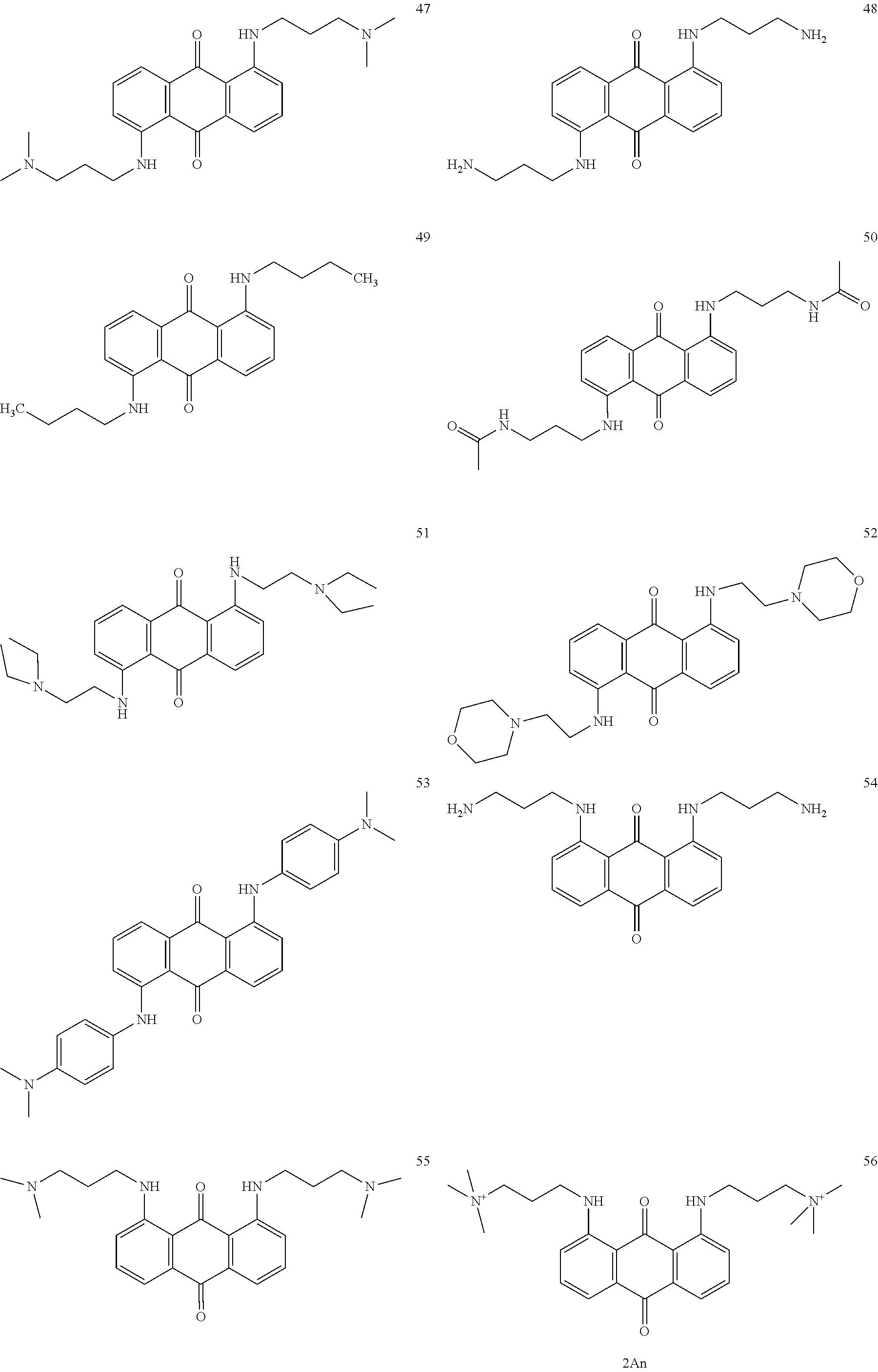

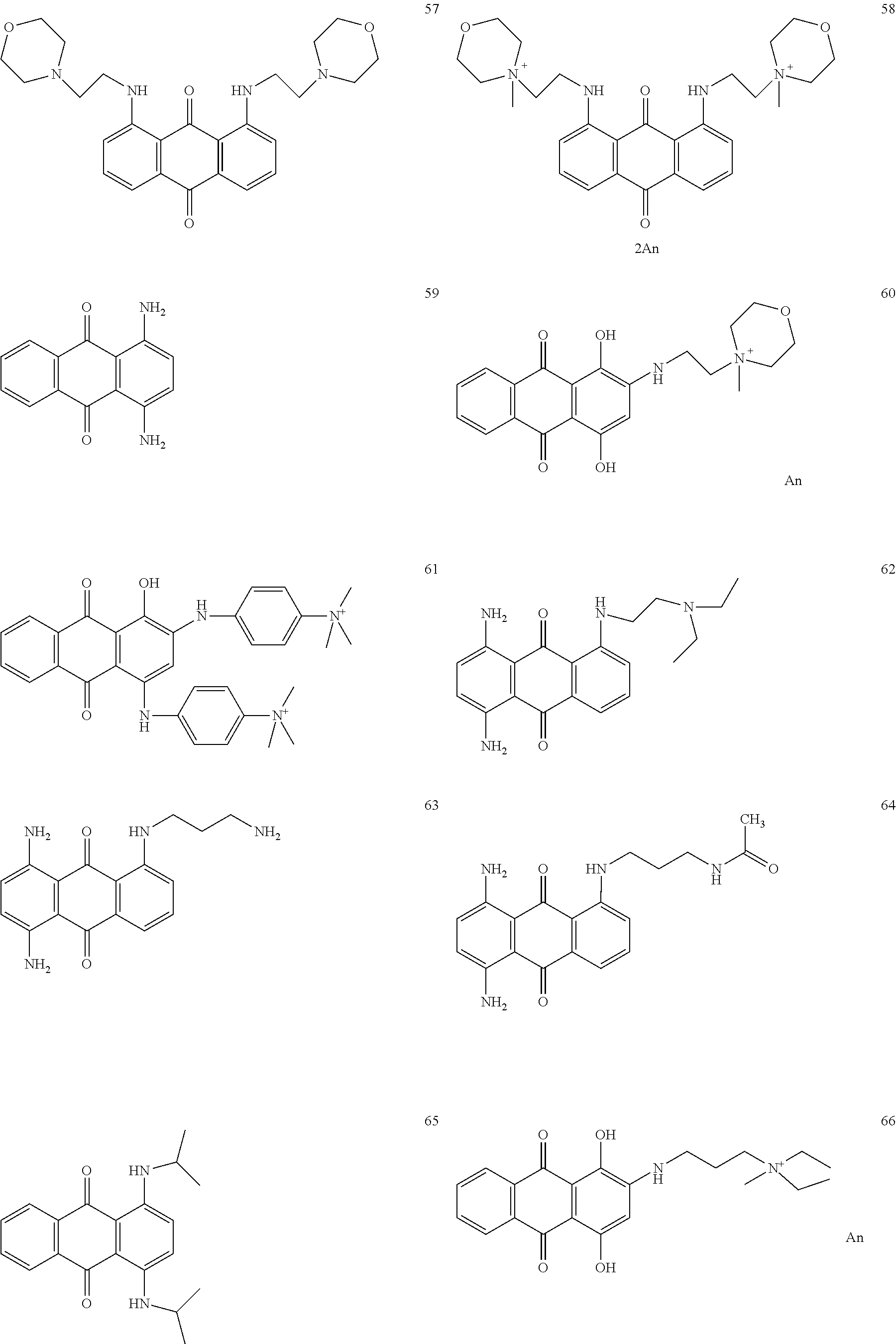

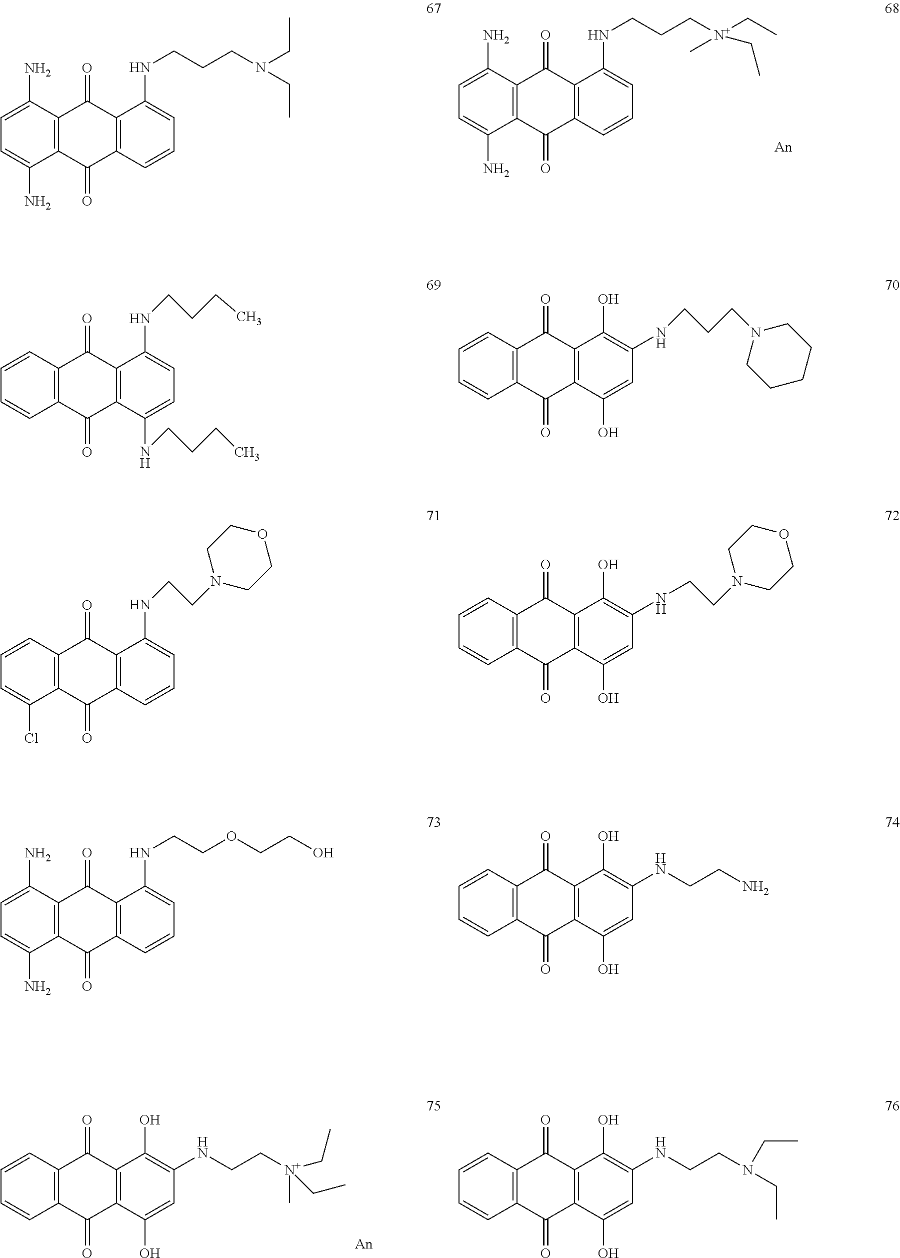

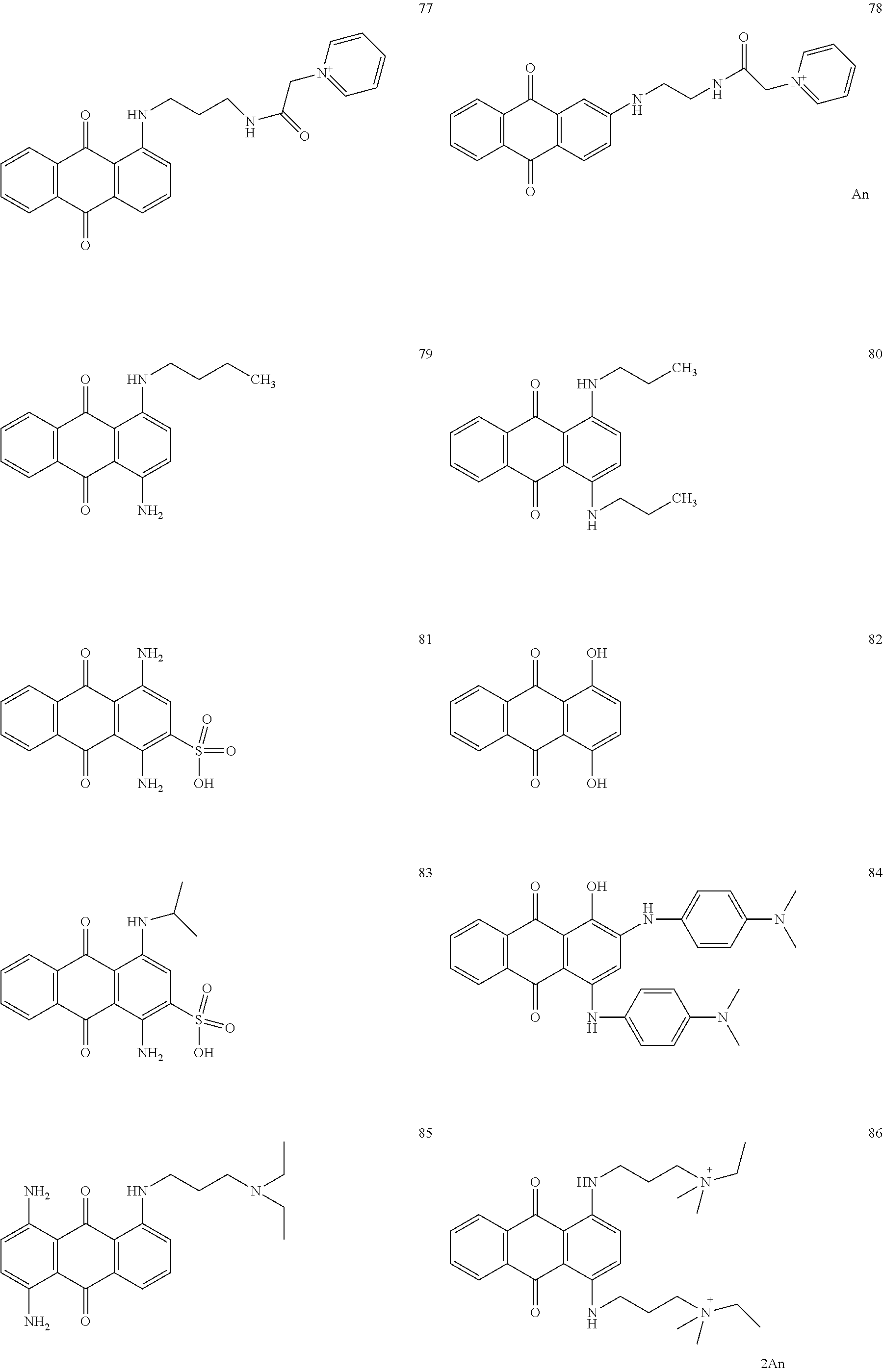

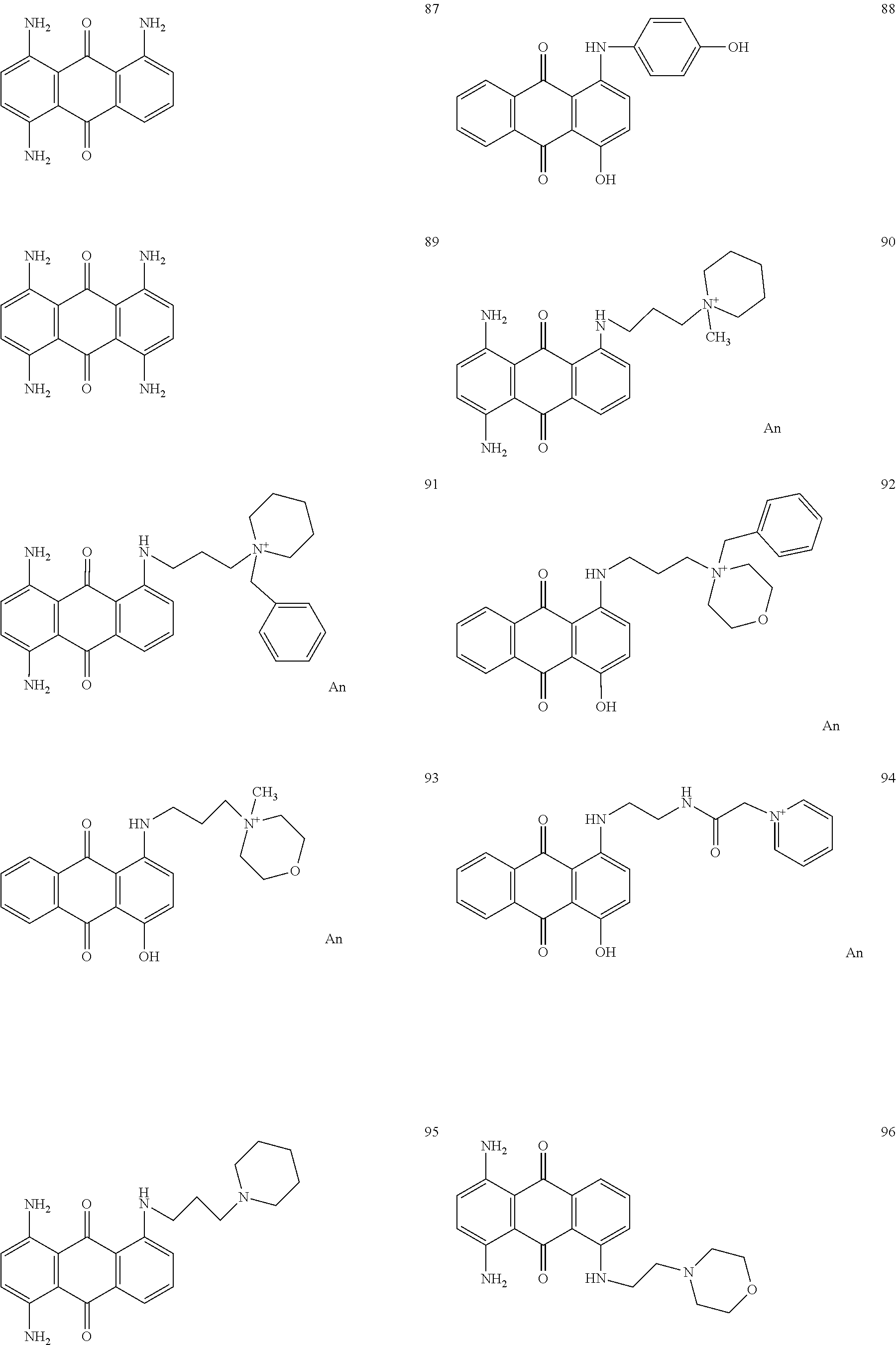

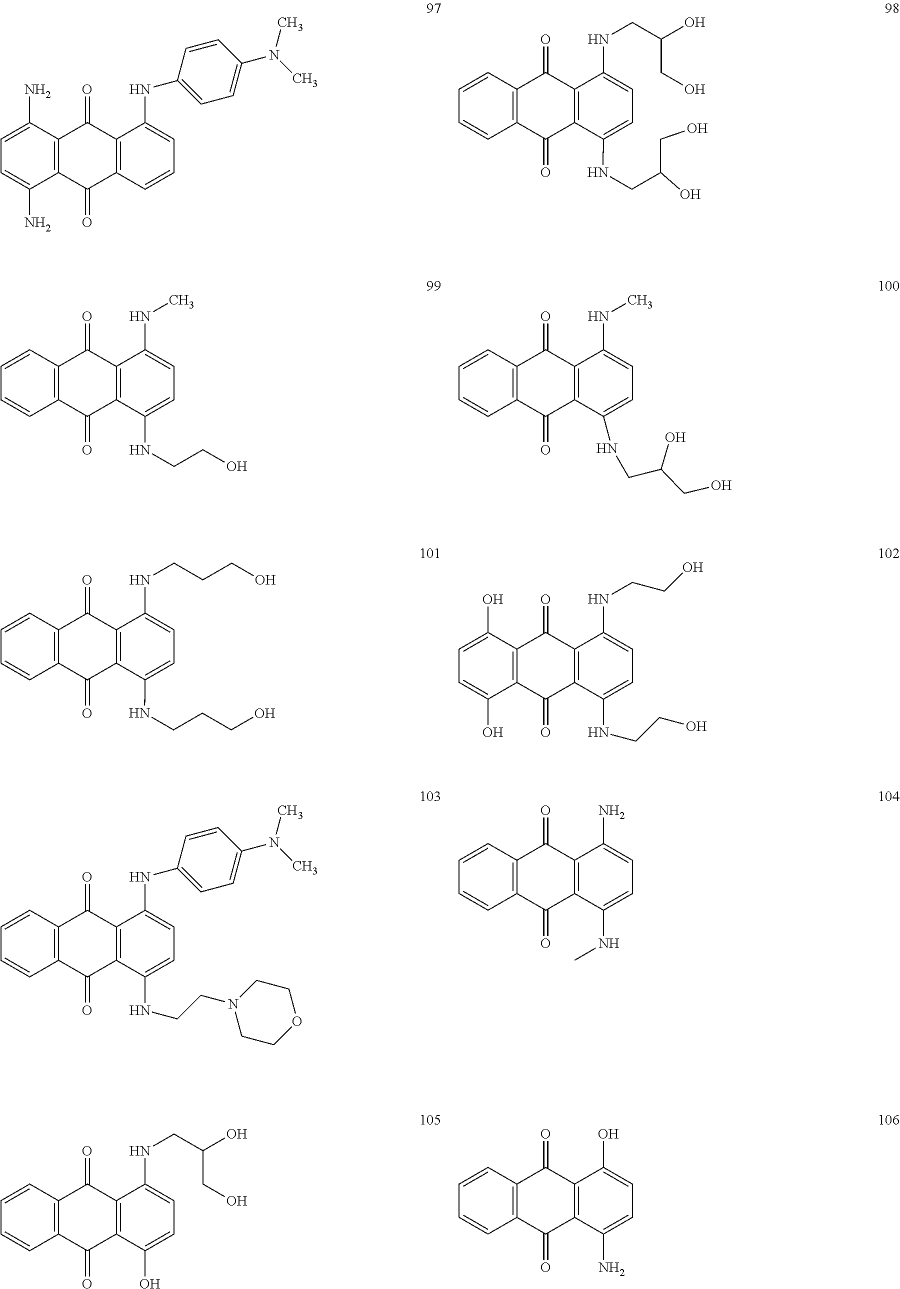

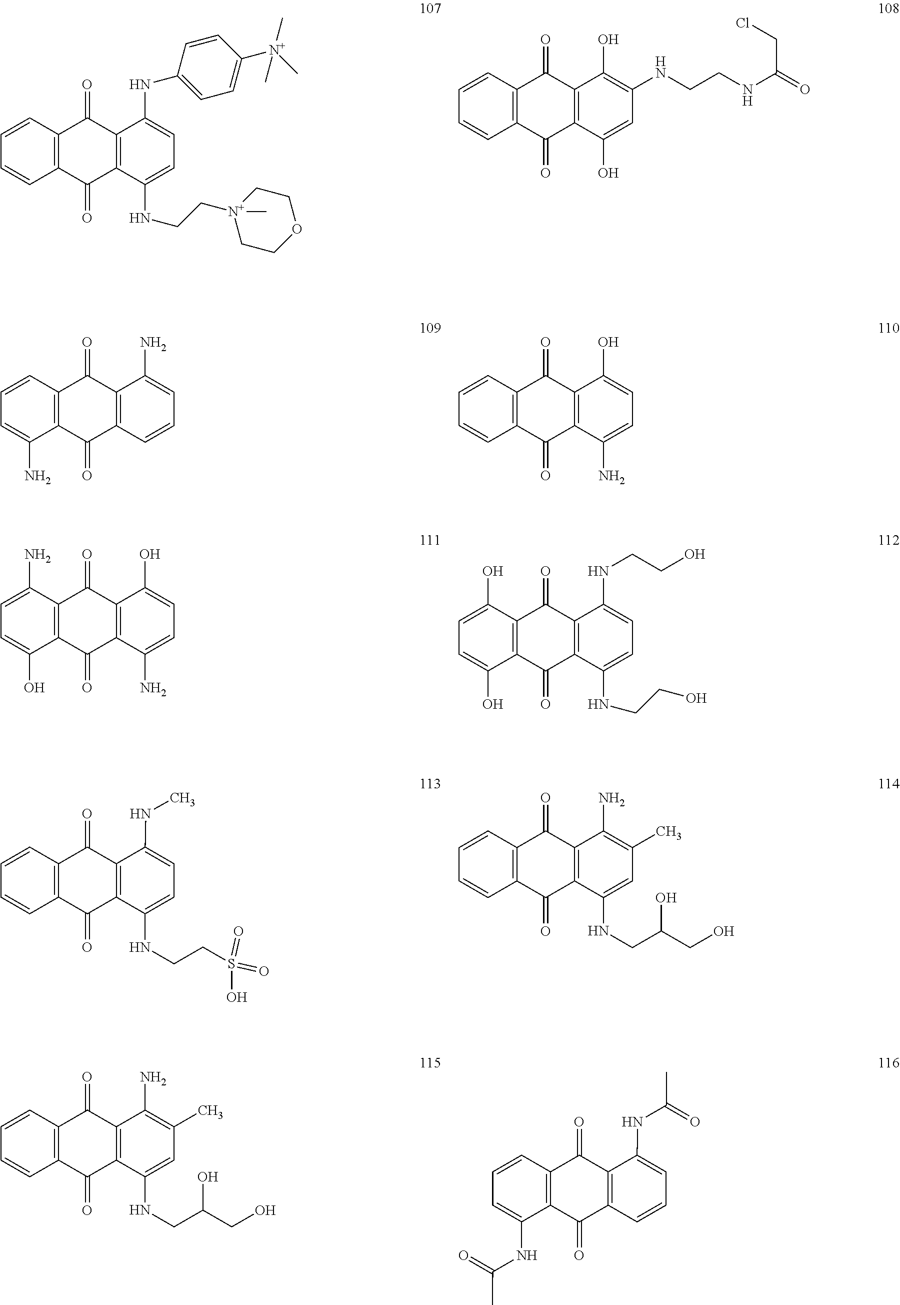

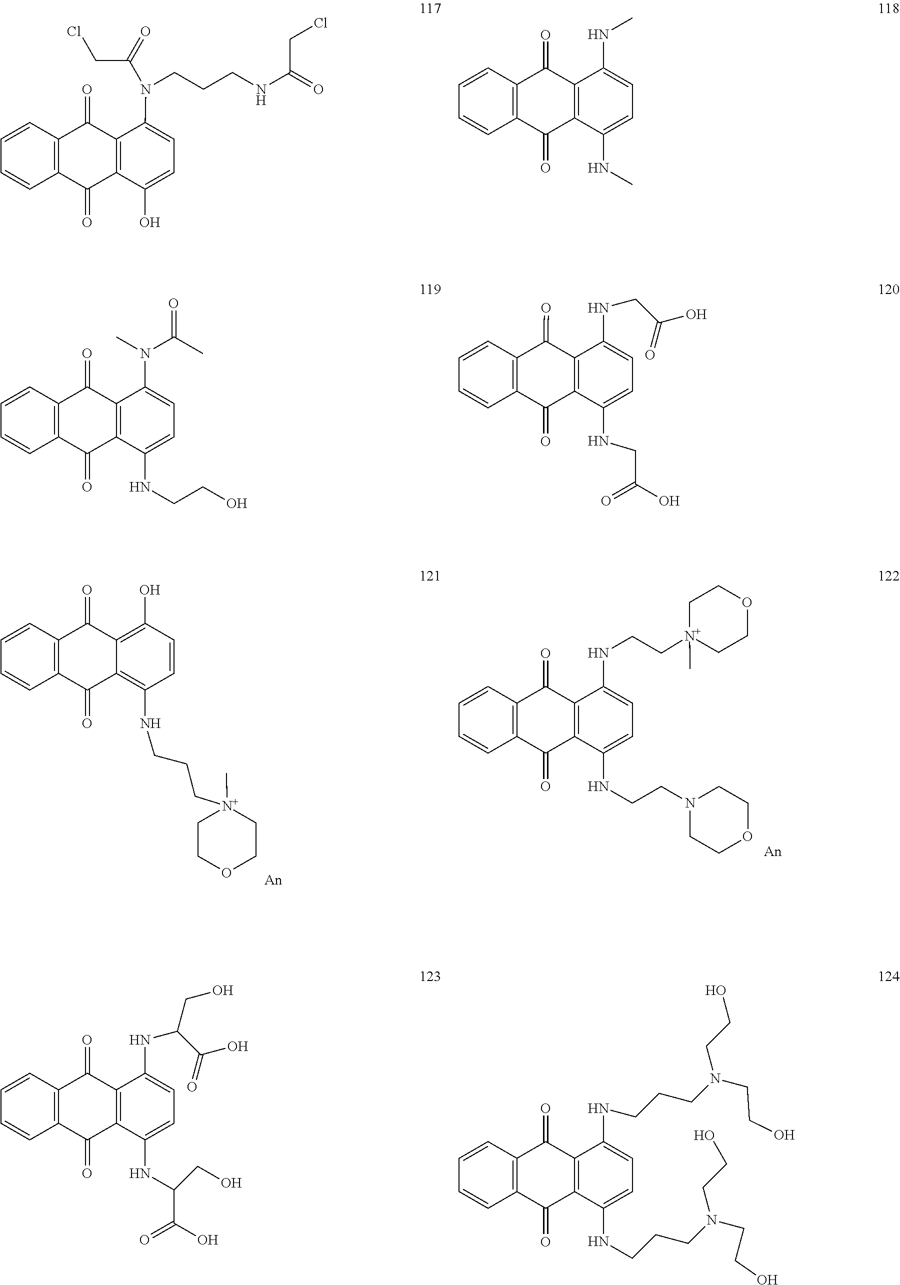

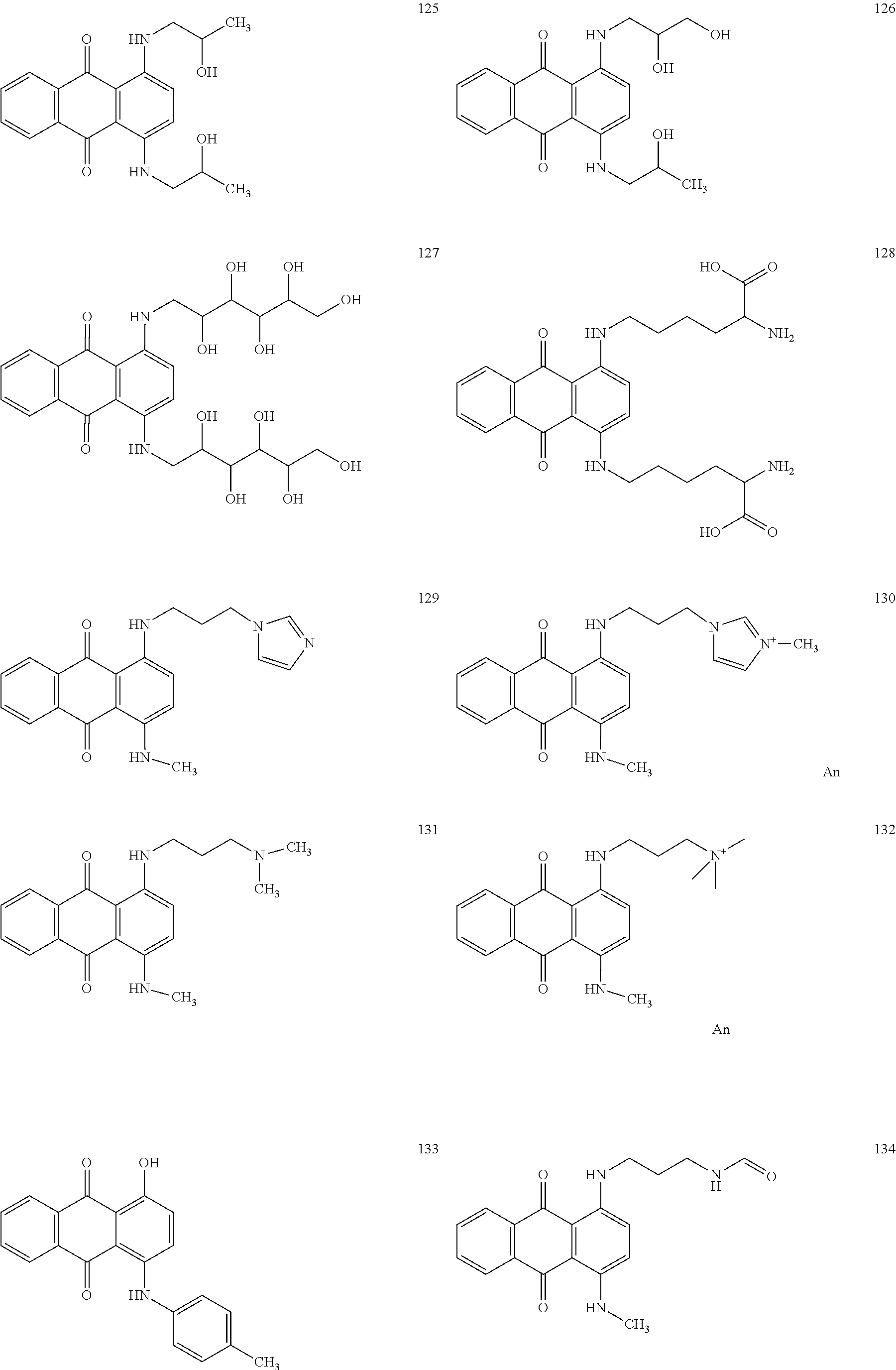

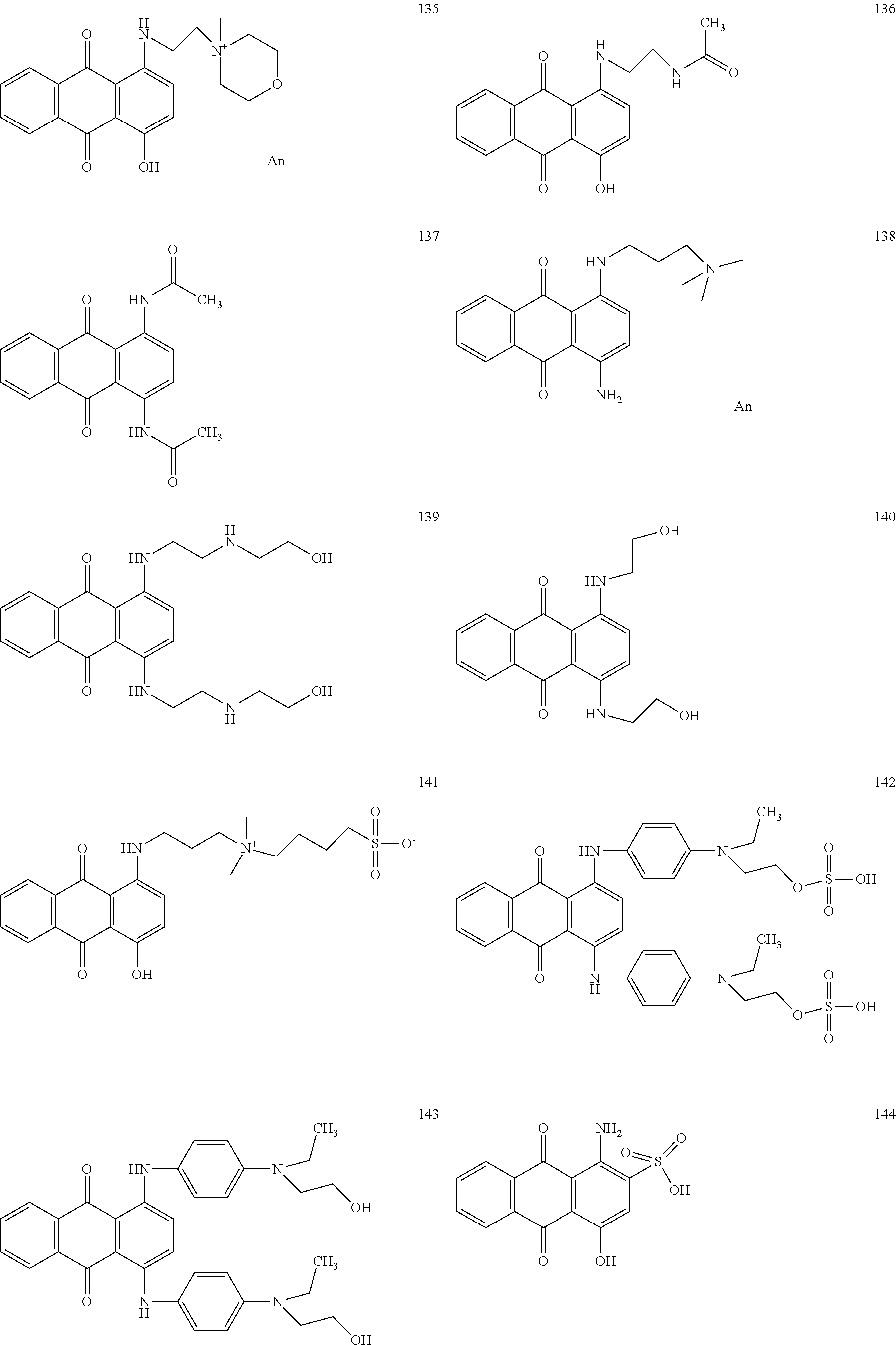

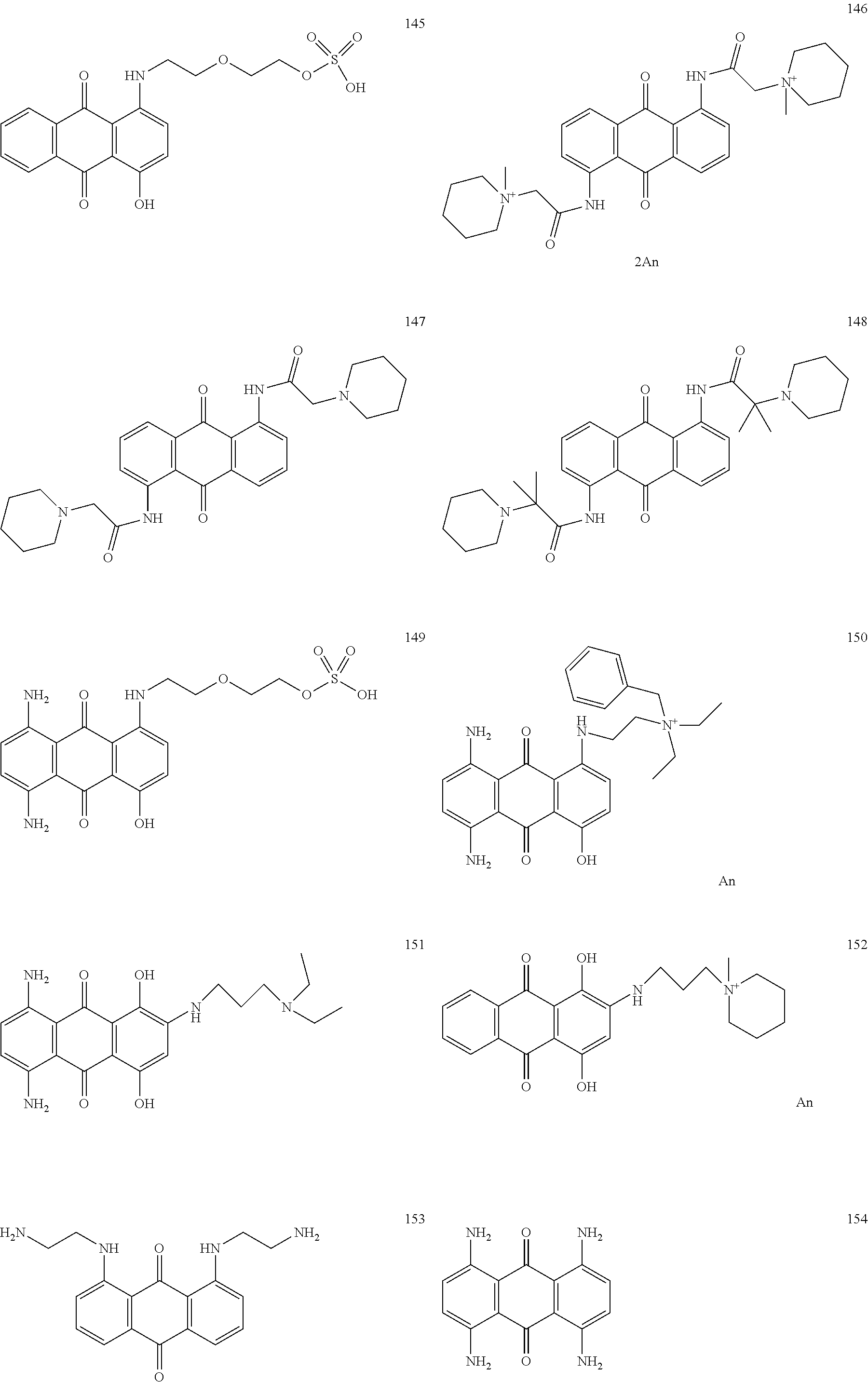

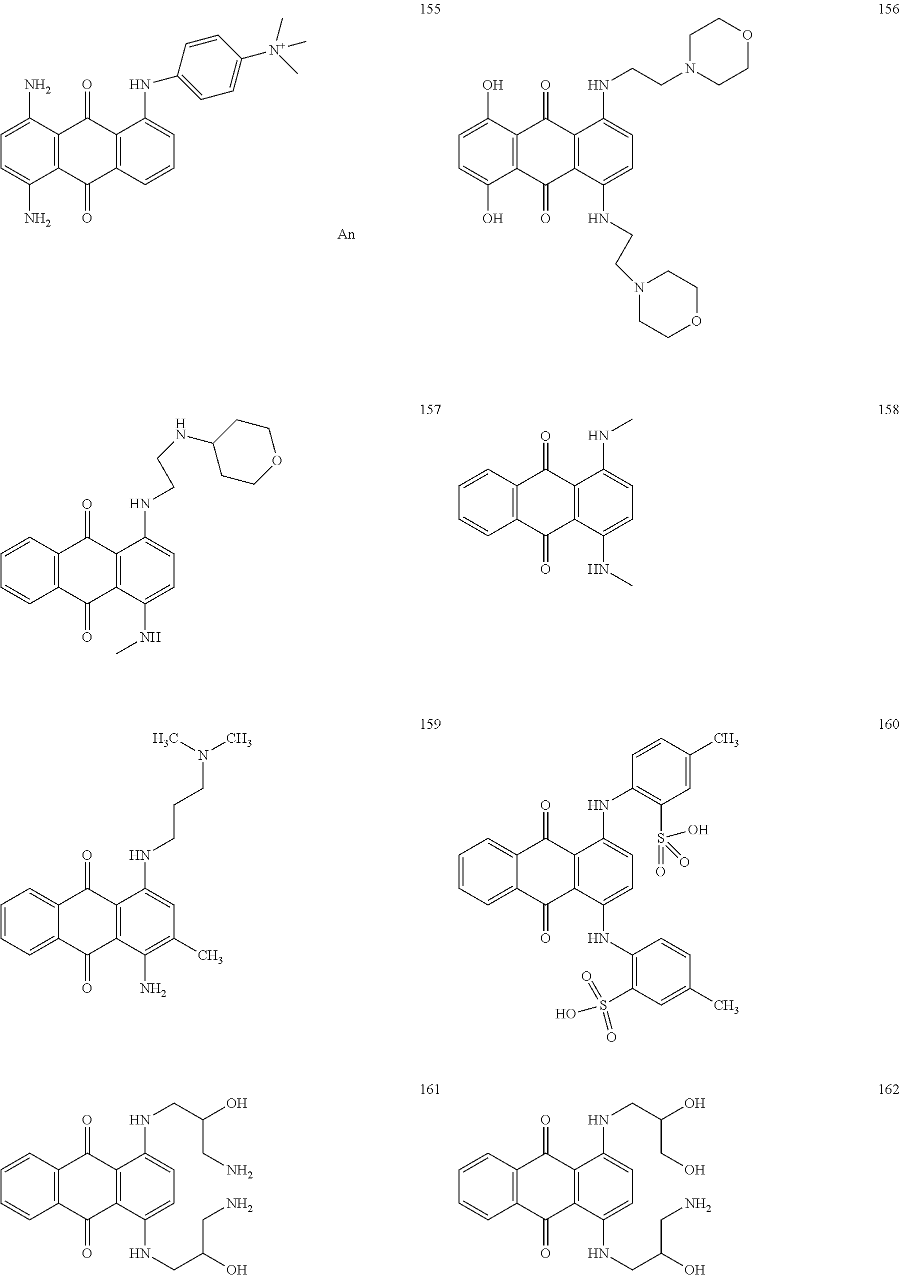

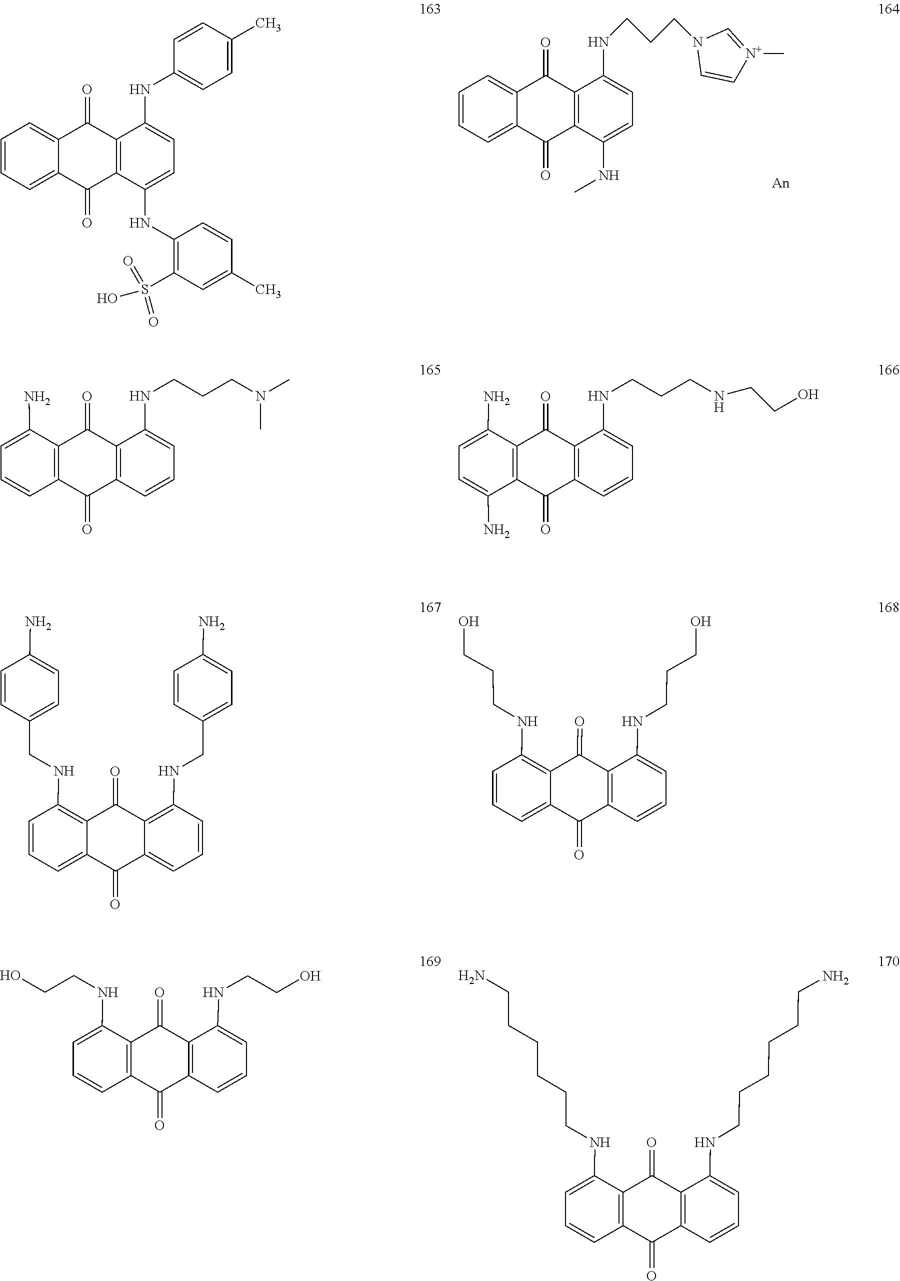

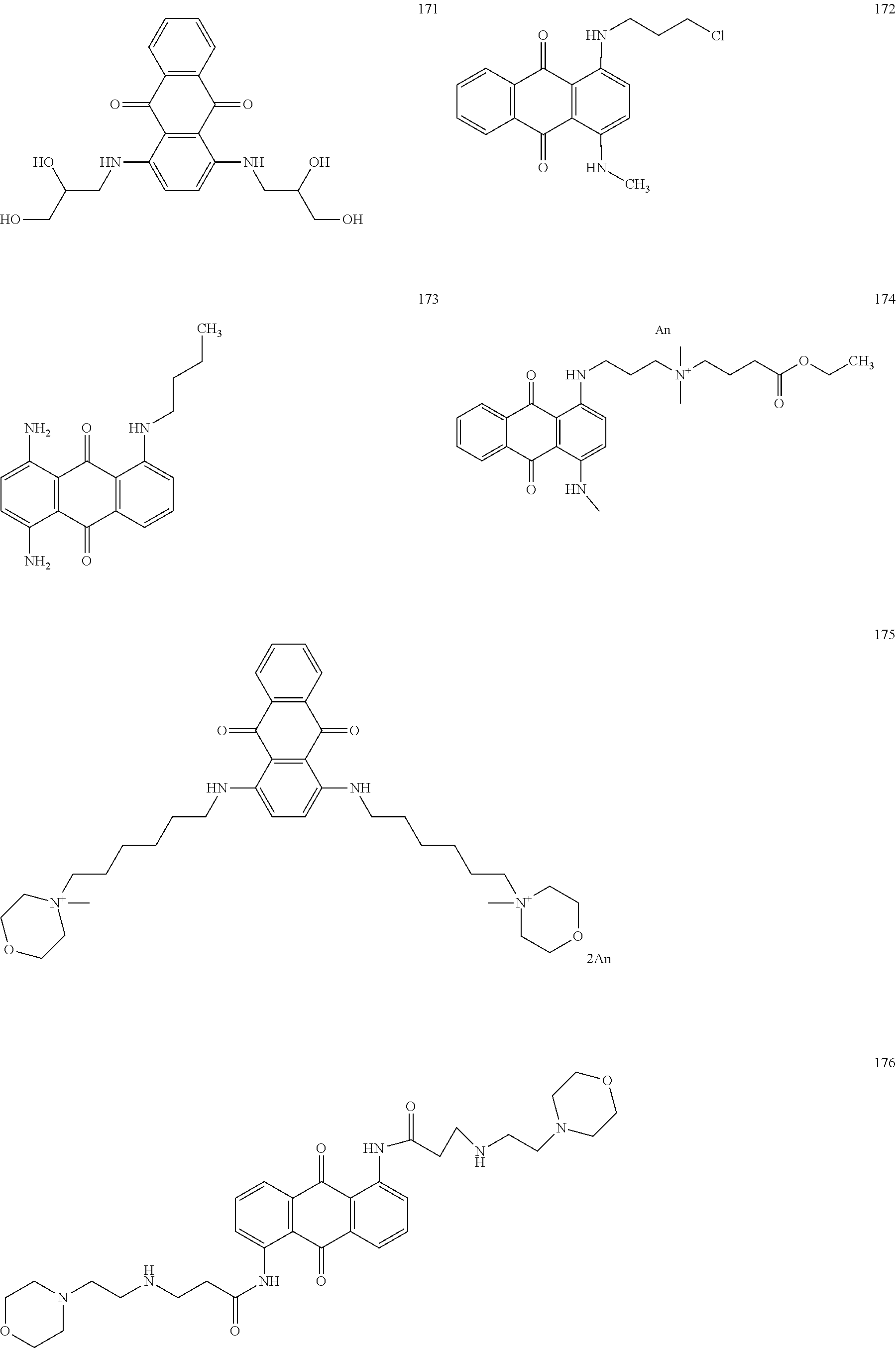 [SMILES: O=C1c2ccccc2C(=O)c2c(NCC(O)CO)ccc(NCC(O)CO)c21 ; CNc1ccc(NCCCCl)c2c1C(=O)c1ccccc1C2=O ; CCCCNc1cccc2c1C(=O)c1c(N)ccc(N)c1C2=O ; CCOC(=O)CCC[N+](C)(C)CCCNc1ccc(NC)c2c1C(=O)c1ccccc1C2=O ; C[N+]1(CCCCCCNc2ccc(NCCCCCC[N+]3(C)CCOCC3)c3c2C(=O)c2ccccc2C3=O)CCOCC1 ; O=C(CCNCCN1CCOCC1)Nc1cccc2c1C(=O)c1cccc(NC(=O)CCNCCN3CCOCC3)c1C2=O]

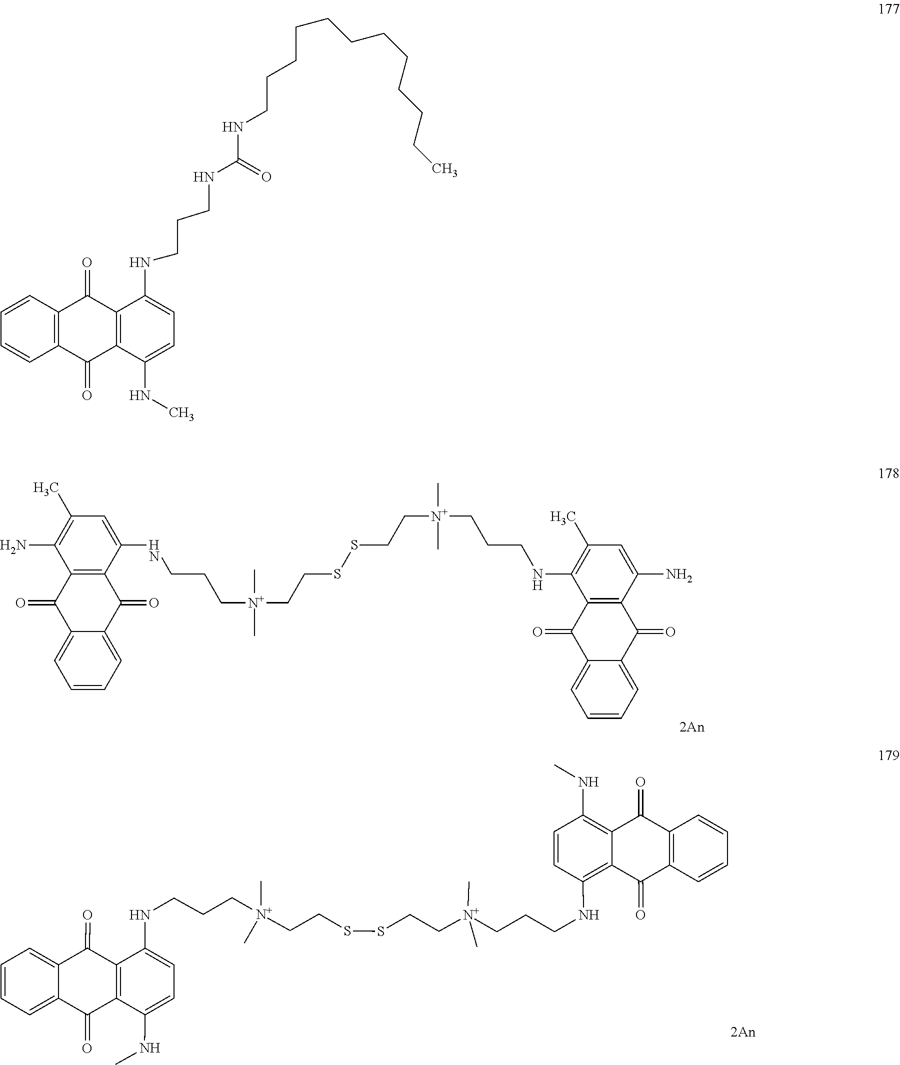 [SMILES: CCCCCCCCCCCCNC(=O)NCCCNc1ccc(NC)c2c1C(=O)c1ccccc1C2=O ; Cc1cc(NCCC[N+](C)(C)CCSSCC[N+](C)(C)CCCNc2c(C)cc(N)c3c2C(=O)c2ccccc2C3=O)c2c(c1N)C(=O)c1ccccc1C2=O ; CNc1ccc(NCCC[N+](C)(C)CCSSCC[N+](C)(C)CCCNc2ccc(NC)c3c2C(=O)c2ccccc2C3=O)c2c1C(=O)c1ccccc1C2=O]

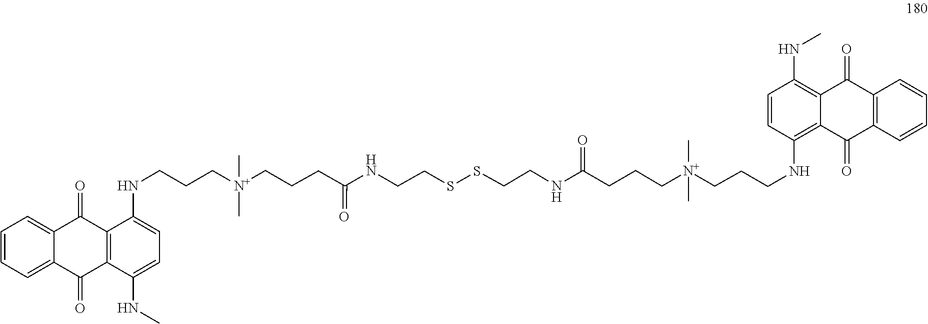 [SMILES: CNc1ccc(NCCC[N+](C)(C)CCCC(=O)NCCSSCCNC(=O)CCC[N+](C)(C)CCCNc2ccc(NC)c3c2C(=O)c2ccccc2C3=O)c2c1C(=O)c1ccccc1C2=O]

with An, which may be identical or different, representing an anionic counterion as defined previously, preferably chosen from mesylate, tosylate and halide such as Cl⁻ and I⁻.

The dyes of formulae (I) and (II) of the invention are known commercial products, or are accessible via standard synthetic routes known to those skilled in the art. Examples that may be mentioned include: Chemical Reviews, “The chemistry of anthraquinones”, 6, pages 157-174 (1926); and Colourage “Blue disperse dyes—structural diversities”, 55 (11), pages 36, 38 and 42 (2008).

(b) Fluorescent Dyes

The process for dyeing keratin fibres according to the present invention and the composition of the invention also use, or comprise, (b) one or more fluorescent dyes. Preferably, the fluorescent dye(s) of the invention are direct dyes. More particularly, the fluorescent dyes of the invention are other than fluorescent dyes comprising a disulfide bond; preferably, the fluorescent dyes of the invention are direct and do not comprise a bond with identical contiguous heteroatoms.

As fluorescent dyes that may be used in the present invention, mention may be made of the following dyes: acridines, acridones, azlactones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), dipyrinones, diketopyrrolopyrroles, fluorindines, (poly)methines (especially cyanins and styryls/hemicyanins), naphthalimides, naphthanilides, naphthylamine (such as dansyls), naphtholactams, oxadiazoles, oxazines, perilones, perinones, perylenes, polyenes/carotenoids, squaranes, stilbenes, xanthenes, thioxanthenes and thiazines. Mention may also be made of the fluorescent dyes described in EP 1 133 975, WO 03/029 359, EP 860 636, WO 95/01772, WO 95/15144, EP 714 954 and those listed in the encyclopaedia The chemistry of synthetic dyes by K. Venkataraman, 1952, Academic Press, vol. 1 to 7, in Kirk Othmer's encyclopaedia Chemical Technology, in the chapter “Dyes and Dye Intermediates”, 1993, Wiley and Sons, and in various chapters of Ullmann's Encyclopedia of Industrial Chemistry 7th edition, Wiley and Sons, especially in Ullmann's Encyclopedia of Industrial Chemistry in the chapter “Fluorescent Dyes”, 2005, Wiley-VCH Verlag GmbH & Co. KGaA, Weinheim 10.1002/143560007.a 1_279; in The Handbook—A Guide to Fluorescent Probes and Labeling Technologies, 10th Ed Molecular Probes/Invitrogen—Oregon 2005 circulated by Internet or in the preceding printed editions.

Preferably, the fluorescent dyes (b) of the invention are chosen from coumarin dyes, (poly)methine dyes, in particular cyanin and styryl/hemicyanin dyes, and naphthalimide dyes. More particularly, the fluorescent dyes of the invention are cationic.

In particular, the fluorescent dye(s) of the invention are direct dyes chosen from (poly)methyl dyes, in particular cyanin dyes and styryl/hemicyanin dyes, and naphthalimide dyes, and mixtures thereof; more particularly, the fluorescent dyes are cationic; preferably, the fluorescent dye(s) (b) are chosen from styryl or hemicyanin cationic dyes.

Better still, the fluorescent dyes of the invention absorb light in the yellow, orange and red range, preferably in the absorption wavelength λ_{abs} between 400 nm and 500 nm inclusive.

According to one variant, the fluorescent dyes of the invention contain at least one cationic radical borne by, or included in, at least one of the fluorescent chromophores.
Preferably, the cationic radical is a quaternary ammonium; better still, the cationic charge is endocyclic.
These cationic radicals are, for example, a cationic radical:
bearing an exocyclic (di/tri)(C₁-C₈)alkylammonium charge, or
bearing an endocyclic charge, such as the following cationic heteroaryl groups: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenoxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

According to a preferred variant of the invention, the fluorescent dyes of the invention bear at least one cationic chromophore and comprise at least one quaternary ammonium radical such as polymethine chromophores chosen from formulae (III) and (IV) below:

W⁺—[C(R^{c})═C(R^{d})]_{m′}—Ar′-(*)Q⁻ (III)

Ar—[C(R^{d})═C(R^{c})]_{m′}—W′⁺-(*)Q⁻ (IV),

in which formulae (III) and (IV):
W⁺ represents a cationic heteroaryl group, in particular comprising a quaternary ammonium optionally substituted with one or more (C₁-C₈)alkyl groups optionally substituted in particular with one or more hydroxyl groups;
W′⁺ represents a divalent heteroaryl radical as defined for W⁺;
Ar represents an aryl group such as phenyl or naphthyl, optionally substituted preferably with i) one or more halogen atoms such as chlorine or fluorine; ii) one or more (C₁-C₈)alkyl and preferably C₁-C₄ alkyl groups such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
Ar′ is a divalent aryl radical as defined for Ar;
m′ represents an integer between 1 and 4 inclusive, in particular, m is 1 or 2; better still 1;
R^{c} and R^{d}, which may be identical or different, represent a hydrogen atom or optionally a substituted (C₁-C₈)alkyl and preferably C₁-C₄ alkyl group, or alternatively R^{c} is contiguous with W or W′ and/or R^{d} is contiguous with Ar or Ar′ and form, with the atoms that bear them, a (hetero)cycloalkyl; in particular, R^{c} is contiguous with W⁺ or W′⁺ and forms a (hetero)cycloalkyl such as cyclohexyl;
Q⁻ is an organic or mineral anionic counterion;
(*) represents the part of the fluorescent chromophore that is bonded to the rest of the dye.

Preferably, W⁺ or W′⁺ is an imidazolium, pyridinium, benzimidazolium, pyrazolium, benzothiazolium or quinolinium radical optionally substituted with one or more identical or different C₁-C₄ alkyl radicals.

According to a particularly preferred embodiment of the invention, the fluorescent chromophore(s) of the dye (III) or (IV) are those defined previously with m′=1, Ar representing a phenyl group substituted para to the styryl group —C(R^{d})═C(R^{c})— with a (di)(hydroxy)(C₁-C₆)(alkyl)amino group such as dihydroxy(C₁-C₄)alkylamino, and W′⁺ representing an imidazolium or pyridinium group, preferentially ortho- or para-pyridinium.

According to another preferred variant of the invention, the fluorescent chromophore(s) of the dyes are cationic and comprise at least one quaternary ammonium radical such as a naphthimidyl bearing a cationic exocyclic charge of formula (IIIa) or (IVa):

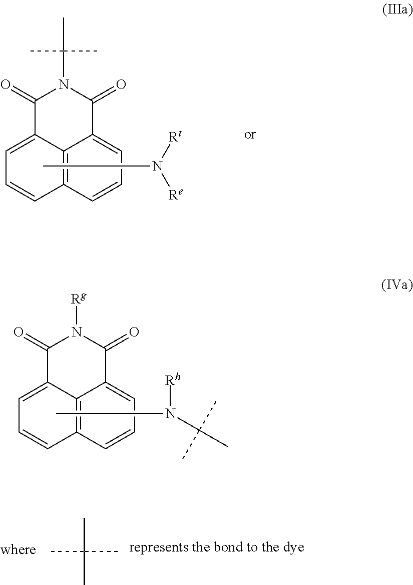

in which formulae (IIIa) and (IVa) R^{e}, R^{f}, R^{g} and R^{h}, which may be identical or different, represent a hydrogen atom or a (C₁-C₆)alkyl group which is optionally substituted, preferably with a di(C₁-C₆)alkylamino or tri(C₁-C₆)alkylammonium group such as trimethylammonium. According to a particular embodiment, the fluorescent dye(s) (b) of the invention are chosen from those of formula (V), (VI) or (VII) below:

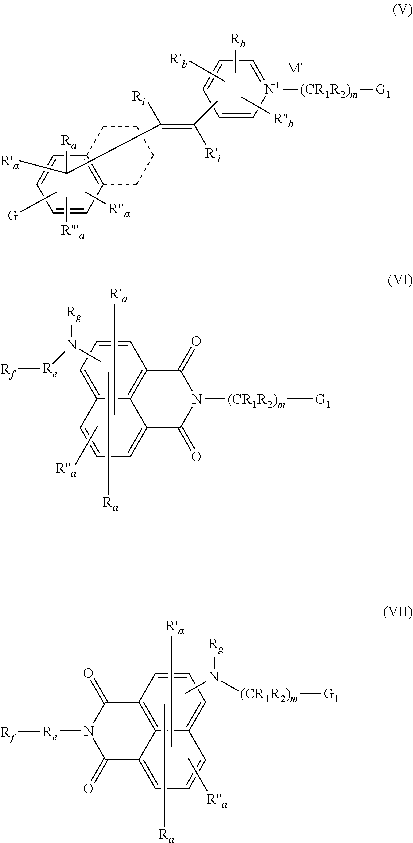

and also the organic or mineral acid or base salts thereof, the optical isomers, geometrical isomers and tautomers thereof, and the solvates thereof such as hydrates; in which formulae (V), (VI) and (VII):
R₁ and R₂, which may be identical or different, represent a hydrogen atom or a C₁-C₆ alkyl group; preferentially a hydrogen atom;
G₁ represents a hydrogen atom or a group chosen from NH₂ and OH preferably hydrogen;
Rₐ, R′ₐ, R″ₐ, R′″ₐ, R_{b}, R′_{b}, R″_{b}, and R′″_{b}, which may be identical or different, represent a) a hydrogen atom, b) a halogen atom, a group from among: c) amino, d) (C₁-C₄)alkylamino, e) (C₁-C₄)dialkylamino, f) cyano, g) carboxyl —C(O)OH or carboxylate —C(O)O⁻, Q⁺, h) hydroxy —OH or alkoxide —O⁻Q⁺, i) (poly)halo(C₁-C₆)alkyl such as trifluoromethyl, j) acylamino, k) (C₁-C₆)alkoxy, l) (C₁-C₆)alkylthio, m) (poly)hydroxy(C₂-C₄)alkoxy, n) (C₁-C₆)alkylcarbonyloxy, o) (C₁-C₆)alkoxycarbonyl, p) (C₁-C₆)alkylcarbonylamino, q) acylamino, r) carbamoyl, s) (C₁-C₆)alkylsulfonylamino, t) aminosulfonyl, u) —SO₃H or sulfonate —SO₃⁻, Q⁺ or v) (C₁-C₆)alkyl optionally substituted with a group chosen from (C₁-C₆)alkoxy, hydroxyl, cyano, carboxyl, amino, (di)(C₁-C₄)alkylamino, or alternatively the two alkyl radicals borne by the nitrogen atom of the amino group form a 5- to 7-membered heterocycle optionally comprising another nitrogen or non-nitrogen heteroatom; particularly, Rₐ, R′ₐ, R″ₐ, R′″ₐ, R_{b}, R′_{b}, R″_{b} and R′″_{b} represent a hydrogen or halogen atom or a (C₁-C₄)alkyl group, preferably a hydrogen atom;
or alternatively two groups Rₐ and R′ₐ; R_{b} and R′_{b}, borne by two adjacent carbon atoms, together form a benzo or indeno ring, a fused heterocycloalkyl or fused heteroaryl group; the benzo, indeno, heterocycloalkyl or heteroaryl ring being optionally substituted with a halogen atom, an amino, (C₁-C₄)alkylamino, (C₁-C₄)dialkylamino, nitro, cyano, carboxyl, hydroxyl or trifluoromethyl group, an acylamino, (C₁-C₄)alkoxy (poly)hydroxy(C₁-C₄)alkoxy, (C₁-C₄)alkylcarbonyloxy, (C₁-C₄)alkoxycarbonyl or (C₁-C₄)alkylcarbonylamino radical, an acylamino, carbamoyl or alkoxyalkylsulfonylamino radical, an aminosulfonyl radical, or a (C₁-C₆)alkyl radical optionally substituted with: a group chosen from (C₁-C₆)alkoxy, hydroxyl, cyano, carboxyl, amino, (C₁-C₄)alkylamino and (C₁-C₄)dialkylamino, or alternatively the two alkyl radicals borne by the nitrogen atom of the amino group form a 5- to 7-membered heterocycle optionally comprising another nitrogen or non-nitrogen heteroatom; preferentially, Rₐ and R′ₐ together form a benzo group;
or alternatively, two groups Rᵢ and Rₐ; and/or a group R′ᵢ and R′ₐ together form a fused (hetero)cycloalkyl, preferentially cycloalkyl such as cyclohexyl;
R_{g} represents a hydrogen atom, a (hetero)aryl(C₁-C₄)alkyl group or a (C₁-C₆)alkyl group that is optionally substituted; preferentially, R_{b} represents a hydrogen atom or a (C₁-C₃)alkyl or benzyl group;
Rₑ represents a covalent bond, a linear or branched, optionally substituted (C₁-C₈)alkylene or (C₂-C₈)alkenylene hydrocarbon-based chain, preferably Rₑ represents an unsubstituted (C₁-C₆)alkylene;
R_{f} represents a hydrogen atom, a (C₁-C₄)alkoxy group, an amino group R₃R₄N—, a quaternary ammonium group M′, R₃R₄R₅N⁺— in which R₃, R₄ and R₅, which may be identical or different, represent a (C₁-C₄)alkyl group or R₃R₄N— represents an optionally substituted heteroaryl group, preferentially an optionally substituted imidazolyl group, or alternatively M′, R₃R₄R₅N⁺— represents an optionally substituted cationic heteroaryl group, preferentially an imidazolinium group optionally substituted with a (C₁-C₃)alkyl group;
G represents a group i) —NR_{c}R_{d}, ii) —OR with R representing a) a hydrogen atom, b) an optionally substituted, preferentially unsubstituted (C₁-C₆)alkyl group, c) an optionally substituted (hetero)aryl group, d) an optionally substituted (hetero)aryl(C₁-C₆)alkyl group such as benzyl, e) optionally substituted (hetero)cycloalkyl group, f) optionally substituted (hetero)cycloalkyl(C₁-C₆)alkyl group; according to a particular embodiment, G represents a group —NR_{c}R_{d}, according to another particular embodiment, G represents a (C₁-C₆)alkoxy group;
   or alternatively when G represents —NR_{c}R_{d}, two groups R_{c} and R′ₐ and/or R_{d} and Rₐ together form a saturated heteroaryl or heterocycle, optionally substituted with one or more (C₁-C₆)alkyl groups, preferentially a 5- to 7-membered heterocycle containing one or two heteroatoms chosen from nitrogen and oxygen; more preferentially, the heterocycle is chosen from morpholinyl, piperazinyl, piperidyl and pyrrolidinyl groups;
R_{c} and R_{d}, which may be identical or different, represent a hydrogen atom or a group from among: a) optionally substituted (hetero)aryl such as phenyl, b) optionally substituted (hetero)aryl(C₁-C₄)alkyl, c) optionally substituted (hetero)cycloalkyl, d) optionally substituted (hetero)cycloalkyl(C₁-C₄)alkyl, f) (C₂-C₅)alkyl or g) (C₁-C₈)alkyl which is optionally substituted, preferably optionally substituted with a hydroxyl, carboxyl, carboxylate, sulfate or sulfonate group;
   or alternatively two adjacent radicals R_{c} and R_{d} borne by the same nitrogen atom together form an optionally substituted heterocyclic or optionally substituted heteroaryl group;
Rᵢ and R′ᵢ, which may be identical or different, represent a hydrogen atom or a (C₁-C₄)alkyl group;

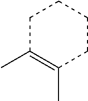

represents a (hetero)aryl group fused to the phenyl ring; or alternatively is absent from the phenyl; preferentially, when the ring is present, the ring is a benzo;
m represents an integer between 1 and 18 inclusive, particularly an integer between 1 and 14 inclusive; preferentially an integer between 2 and 10 inclusive; more preferentially an integer between 3 and 8; more particularly an integer between 4 and 6;
M′ represents an anionic counterion, derived from a salt of an organic or mineral acid, or from an organic or mineral base that ensures the electrical neutrality of the molecule;
Q⁺ represents a cationic counterion, derived from a salt of an organic or mineral acid, or from an organic or mineral base that ensures the electrical neutrality of the molecule such as alkali metal, alkaline-earth metal or ammonium;
   it being understood that when the molecule comprises a carboxylate, sulfonate or alkoxide group, then M′ and Q⁺ may be absent to ensure the electrical neutrality of said molecule.
   According to one embodiment, the fluorescent dyes of the invention are of formula (V) as defined previously.

According to a preferred embodiment, the fluorescent dyes of the invention are chosen from the styryl dyes of formula (VIII) below:

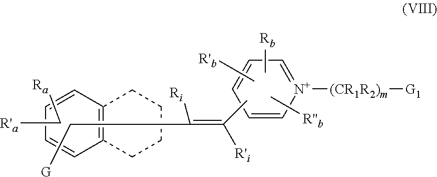

and also the organic or mineral acid or base salts thereof, the optical isomers, geometrical isomers and tautomers thereof, and the solvates thereof such as hydrates;
in which formula (VIII) G, G₁, Rₐ, R′ₐ, R″ₐ, R_{b}, R′_{b}, R″_{b}, Rᵢ, R′ᵢ, R₁, R₂ and m are as defined previously for (V).

In particular, the dye(s) of the invention are chosen from those of formula (VIII) for which:
R₁ and R₂, which may be identical or different, represent a hydrogen atom;
Rᵢ and R_{i′}, which may be identical or different, represent a hydrogen atom or a (C₁-C₄)alkyl group, preferably hydrogen;
Rₐ, R′ₐ and R″ₐ, which may be identical or different, represent a hydrogen atom, a halogen atom such as fluorine, or an —OH, —O⁻Q⁺, (C₁-C₆)alkoxy, nitro, or cyano group, with Q⁺ as defined previously;
R_{b}, R′_{b} and R″_{b}, which may be identical or different, represent a hydrogen atom or a (C₁-C₆)alkyl group;
or alternatively two contiguous radicals R_{b} and R′_{b} form, together with the carbon atoms that bear them, a benzo group that is condensed or fused to the pyridinium group, said benzo group possibly being substituted; preferably, said benzo group is unsubstituted;
G represents a group —NR_{c}R_{d} or (C₁-C₆)alkoxy group which is optionally substituted, preferentially unsubstituted; according to a particular embodiment, G represents a group —NR_{c}R_{d}, according to another particular embodiment, G represents a (C₁-C₆)alkoxy group;
G₁ represents a hydrogen atom or an OH or NH₂ group, preferably hydrogen;
Rᵢ and R′ᵢ, which may be identical or different, represent a hydrogen atom or a (C₁-C₄)alkyl group;

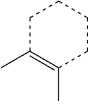

represents an aryl or heteroaryl group fused to the phenyl ring; or alternatively is absent from the phenyl ring; preferentially, when the ring is present, the ring is a benzo;
m represents an integer between 1 and 18 inclusive; particularly an integer between 2 and 16 inclusive; preferentially an integer between 3 and 10; more preferentially an integer between 4 and 6;
R_{c} and R_{d}, which may be identical or different, represent a hydrogen atom, a (C₂-C₄)alkyl group or a substituted (C₁-C₈)alkyl group, preferably (C₂-C₄)alkyl substituted in particular with one or more groups chosen from i) cyano, ii) (C₁-C₃)alkoxy, iii) hydroxyl and iv) (C₁-C₃)alkylcarbonyl, preferably with one or more hydroxyl groups; and
M′ represents an anionic counterion, derived from a salt of an organic or mineral acid, or from an organic or mineral base that ensures the electrical neutrality of the molecule;
   it being understood that when the molecule comprises an alkoxide group, then M′ and Q⁺ may be absent to ensure the electrical neutrality of said molecule.
   Preferably, the fluorescent dye(s) of the invention are chosen from the styryl dyes of formula (IX) below:

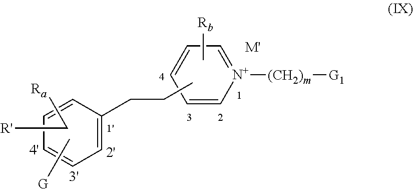

and also the organic or mineral acid salts thereof, the optical isomers, geometrical isomers and tautomers thereof, and the solvates thereof such as hydrates;
in which formula (IX) G, G₁, Rₐ, R′ₐ, R_{b}, R′_{b} and m are as defined previously.
According to a particular embodiment, the group G is in the para position relative to the —CH═CH— group, i.e. in position 4′ of the phenyl group. According to another particular embodiment of the invention, the group G is in the ortho position relative to the —CH═CH— group, i.e. in position 2′ of the phenyl group. According to one embodiment, the —CH═CH— group is in the para position of the pyridinium group, i.e. in position 4.
According to another advantageous variant, the —CH═CH— group is in the ortho position of the pyridinium group, i.e. in position 2.
According to a preferred embodiment of the invention, the fluorescent dyes of the invention (b) are chosen from the compounds of formulae (X) and (XI) below:

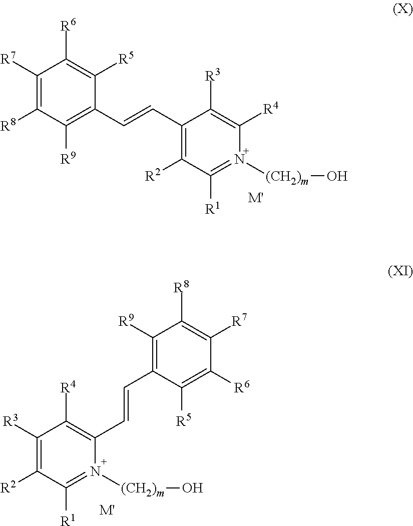

and also the organic or mineral acid salts thereof, the optical isomers, geometrical isomers and tautomers thereof, and the solvates thereof such as hydrates; in which formulae (X) and (XI):
R¹, R², R³ and R⁴, which may be identical or different, represent a hydrogen atom or a (C₁-C₆)alkyl group; preferably, R² and R³ represent a hydrogen atom and R¹ and R⁴, which may be identical or different, represent a hydrogen atom or a (C₁-C₄)alkyl group;
R⁵, R⁶, R⁷, R⁸ and R⁹, which may be identical or different, represent i) a hydrogen atom or ii) a halogen atom such as Cl, Br or F, iii) a group OR in which R represents a hydrogen atom or Q⁺ as described previously, or a (C₁-C₃)alkyl group, iv) aryl such as benzene, v) aryl(C₁-C₃)alkyl such as benzyl, vi) cyano, vii) nitro, viii) (C₁-C₃)alkylthio, ix) amino NR¹⁰R¹¹ with R¹⁰ and R¹¹, which may be identical or different, representing a) a hydrogen atom, b) a (C₂-C₄)alkyl group or c) a substituted (C₁-C₈)alkyl group, preferably (C₂-C₄)alkyl optionally substituted with one or more groups chosen from:
   cyano,
   (C₁-C₃)alkoxy,
   hydroxyl, and
   (C₁-C₃)alkylcarbonyl;
      in particular, R¹⁰ and R¹¹, which may be identical or different, represent a hydrogen atom or a (C₁-C₆)alkyl group substituted with one or more hydroxyl, cyano or (C₁-C₃)alkylcarbonyl groups such as hydroxyethyl;
m represents an integer between 1 and 18 inclusive; particularly an integer between 2 and 16 inclusive; preferentially an integer between 3 and 10; more preferentially an integer between 4 and 6;
M′ represents an anionic counterion as defined previously;
   it being understood that when the molecule comprises an alkoxide group, then M′ and Q⁺ may be absent to ensure the electrical neutrality of said molecule.
   According to one embodiment of the invention, the fluorescent dye(s) (b) are of formula (X) with:

| R¹ | R² | R³ | R⁴ | R⁵ | R⁶ | R⁷ | R⁸ | R⁹ | m |
|---|---|---|---|---|---|---|---|---|---|
| H | H | H | H | H | H | H | H | H | 1 |
| H | H | H | H | H | t-Bu | OH | t-Bu | H | 5 |
| H | H | H | H | H | t-Bu | OH | t-Bu | H | 5 |
| H | H | H | H | H | H | NH₂ | H | H | 1 |
| H | H | H | H | H | H | NH₂ | H | OCH₃ | 1 |
| H | H | H | H | H | H | OH | Br | H | 5 |
| H | H | H | H | H | OCH₃ | OH | OCH₃ | H | 5 |
| H | H | H | H | Cl | H | OH | H | Cl | 5 |
| H | H | H | H | H | H | OH | H | H | 10 |
| H | H | H | H | H | OCH₃ | OH | OCH₃ | H | 10 |
| H | H | H | H | H | t-Bu | OH | t-Bu | H | 10 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | t-Bu | OH | t-Bu | H | 10 |
| H | H | H | H | H | H | N(CH₂CH₃)CH₂CH₂OH | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | n-C₆H₁₃ | H | H | 1 |
| H | H | H | H | H | H | N(CH₂CH₂OH)₂ | H | H | 2 |
| H | H | H | H | H | H | N(n-Bu)₂ | H | H | 2 |
| H | H | H | H | H | OCH₃ | OH | H | H | 10 |
| H | H | H | H | H | H | OC₂H₅OH | H | H | 1 |
| H | H | H | H | H | H | OH | H | H | 1 |

| | | | | | | | | |
|---|---|---|---|---|---|---|---|---|
| H | H | benzo | H | H | H | H | H | 1 |
| H | H | benzo | H | H | N(CH₂CH₂OH)₂ | H | H | 1 |
| H | H | benzo | H | H | N(CH₂CH₂OH)₂ | H | H | 1 |

and also the organic or mineral acid or base salts thereof, the optical isomers, geometrical isomers and tautomers thereof, and the solvates thereof such as hydrates.
According to one embodiment, the fluorescent dye(s) (b) are of formula (XI) with:

| R¹ | R² | R³ | R⁴ | R⁵ | R⁶ | R⁷ | R⁸ | R⁹ | m |
|---|---|---|---|---|---|---|---|---|---|
| CH₃ | H | H | H | OH | H | OCH₃ | H | H | 2 |
| CH₃ | H | H | H | H | H | OCH₃ | OCH₂—Ph | H | 2 |
| CH₃ | H | H | H | H | H | H | H | OCH₃ | 2 |
| CH₃ | H | H | H | F | H | H | H | H | 2 |
| CH₃ | H | H | H | H | H | H | OPh | H | 2 |
| CH₃ | H | H | H | H | H | N(CH₂CH₂OAc)₂ | H | H | 2 |
| CH₃ | H | H | H | OCH₃ | H | OCH₃ | OCH₃ | H | 2 |
| CH₃ | H | H | H | H | H | H | CH₃ | H | 2 |
| CH₃ | H | H | H | H | H | OH | H | H | 2 |
| CH₃ | H | H | H | H | OCH₃ | OCH₃ | OCH₃ | H | 2 |
| CH₃ | H | H | H | H | OCH₃ | OH | OH | H | 2 |
| CH₃ | H | H | H | H | H | OCH₃ | OCH₃ | OCH₃ | 2 |
| CH₃ | H | H | H | H | H | H | OCH₃ | OH | 2 |
| CH₃ | H | H | H | H | H | N(n-butyl)₂ | H | H | 2 |
| CH₃ | H | H | H | H | OCH₃ | OCH₃ | H | H | 2 |
| CH₃ | H | H | H | H | H | H | H | H | 2 |
| CH₃ | H | H | H | H | H | i-propyl | H | H | 2 |
| H | H | H | H | H | H | OH | H | H | 6 |
| H | H | H | H | H | OCH₃ | OCH₃ | OCH₃ | H | 6 |
| H | H | H | H | OH | H | OCH₃ | H | H | 2 |
| H | H | H | H | OCH₃ | H | H | OCH₃ | OCH₃ | 2 |
| H | H | H | H | H | H | H | H | Br | 2 |
| H | H | H | H | H | H | OH | H | H | 2 |
| H | H | H | H | OCH₃ | OCH₃ | OCH₃ | H | H | 6 |
| H | H | H | H | OH | OCH₃ | H | H | H | 6 |
| H | H | H | H | H | OCH₃ | OCH₃ | H | H | 6 |
| H | H | H | H | H | OCH₃ | OCH₃ | H | H | 2 |
| H | H | H | H | H | H | C(O)—OH | H | H | 6 |
| H | H | H | H | H | H | C(O)—OH | H | H | 2 |
| H | H | H | H | H | H | i-propyl | H | H | 2 |
| H | H | H | H | H | H | N(CH₃)CH₂CH₂CN | H | H | 2 |
| H | H | H | H | H | H | OCH₃ | OCH₂Ph | H | 2 |
| H | H | H | H | H | H | H | OPh | H | 2 |
| H | H | H | H | H | H | N(CH₂CH₂C(O)CH₃)₂ | H | H | 2 |
| H | H | H | H | OH | H | OCH₃ | H | H | 6 |
| H | H | H | H | H | OCH₃ | OCH₃ | OCH₃ | H | 2 |
| H | H | H | H | OCH₃ | H | OCH₃ | OCH₃ | H | 6 |
| H | H | H | H | H | H | H | CH₃ | H | 2 |
| H | H | H | H | H | H | N(CH₃)CH₂CH₂OH | H | H | 2 |

and also the organic or mineral acid or base salts thereof, the geometrical isomers and tautomers thereof, and the solvates thereof such as hydrates.
Another subject of the invention is novel dyes chosen from those of formulae (X′) and (XI′) below:

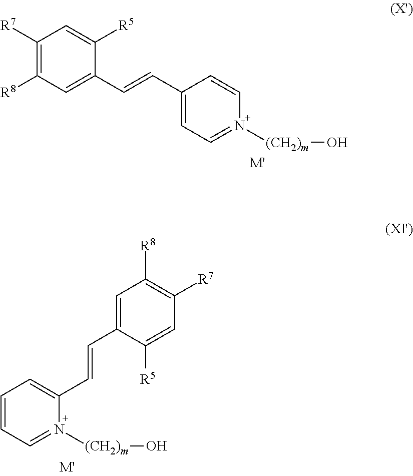

in which formulae (X′) and (XI′) R⁵, R⁷, R⁸ and m are as defined previously for (X) and (XI), in particular:
R⁵ and R⁸, which may be identical or different, represent a hydrogen atom or a (C₁-C₄)alkoxy group such as methoxy;
R⁷ represents a (C₁-C₄)alkoxy group or NR¹⁰R¹¹ with R¹⁰ representing a) a hydrogen atom, or b) a (C₁-C₆)alkyl group substituted with one or more groups chosen from i) cyano, ii) (C₁-C₃)alkoxy, iii) hydroxyl and iv) (C₁-C₃)alkylcarbonyl and R¹¹ representing a) a (C₂-C₅)alkyl group substituted with one or more groups chosen from i) cyano, ii) (C₁-C₃)alkoxy, iii) hydroxyl and iv) (C₁-C₃)alkylcarbonyl;
   in particular, NR¹⁰R¹¹ represents a (C₂-C₄)alkyl group, a (di)hydroxy(C₂-C₄)alkylamino or hydroxy(C₂-C₄)alkyl((C₁-C₄)alkyl)amino group;
m represents an integer between 1 and 18 inclusive; particularly an integer between 2 and 16 inclusive; preferentially an integer between 3 and 10; more preferentially an integer between 4 and 6; and
M′ represents an anionic counterion, derived from a salt of an organic or mineral acid, or from an organic or mineral base that ensures the electrical neutrality of the molecule.
   According to one embodiment, the fluorescent dye(s) are of formula (X′) or (XI′) with:

| R⁵ | R⁷ | R⁸ | m | | R⁵ | R⁷ | R⁸ | m |
|---|---|---|---|---|---|---|---|---|
| H | N(CH₂CH₂OH)₂ | H | 2 | and | OCH₃ | OCH₃ | OCH₃ | 2 |
| H | N(CH₂CH₂OH)₂ | H | 3 | | OCH₃ | OCH₃ | OCH₃ | 3 |
| H | N(CH₂CH₂OH)₂ | H | 4 | | OCH₃ | OCH₃ | OCH₃ | 3 |
| H | N(CH₂CH₂OH)₂ | H | 5 | | OCH₃ | OCH₃ | OCH₃ | 4 |
| H | N(CH₂CH₂OH)₂ | H | 6 | | OCH₃ | OCH₃ | OCH₃ | 5 |
| H | N(CH₂CH₂OH)₂ | H | 8 | | OCH₃ | OCH₃ | OCH₃ | 8 |
| H | N(CH₂CH₂OH)₂ | H | 10 | | OCH₃ | OCH₃ | OCH₃ | 10 |
| H | N(CH₂CH₂OH)₂ | H | 12 | | OCH₃ | OCH₃ | OCH₃ | 12 |
| H | N(CH₂CH₂OH)₂ | H | 14 | | OCH₃ | OCH₃ | OCH₃ | 14 |
| H | N(CH₂CH₂OH)₂ | H | 16 | | OCH₃ | OCH₃ | OCH₃ | 16 |
| H | CH₃CH₂N(CH₂CH₂OH) | H | 2 | | | | | |
| H | CH₃CH₂N(CH₂CH₂OH) | H | 4 | | | | | |

and also the organic or mineral acid or base salts thereof, the geometrical isomers and tautomers thereof, and the solvates thereof such as hydrates.

According to a preferred embodiment of the invention, the fluorescent dyes (b) of the invention are chosen from the compounds of formulae (XII) and (XIII) below:

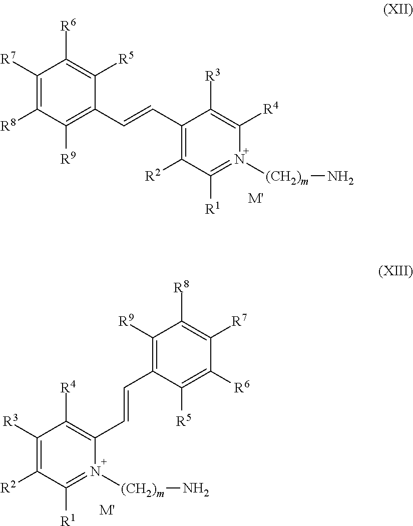

and also the organic or mineral acid salts thereof, the optical isomers, geometrical isomers and tautomers thereof, and the solvates thereof such as hydrates;
in which formulae (XII) and (XIII):
R¹, R², R³ and R⁴, which may be identical or different, represent a hydrogen atom or a (C₁-C₆)alkyl group; preferably, R² and R³ represent a hydrogen atom and R¹ and R⁴, which may be identical or different, represent a hydrogen atom or a (C₁-C₄)alkyl group;
R⁵, R⁶, R⁷, R⁸ and R⁹, which may be identical or different, represent i) a hydrogen atom or ii) a halogen atom such as Cl, Br or F, iii) a group OR in which R represents a hydrogen atom or Q⁺ as described previously, or a (C₁-C₃)alkyl group, iv) aryl such as benzene, v) an aryl(C₁-C₃)alkyl group such as benzyl, vi) cyano, vii) nitro, viii) (C₁-C₃)alkylthio, ix) amino NR¹⁰R¹¹ with R¹⁰ and R¹¹, which may be identical or different, representing a) a hydrogen atom or b) a (C₁-C₈)alkyl group optionally substituted with one or more groups chosen from:
   cyano,
   (C₁-C₃)alkoxy,
   hydroxyl, and
   (C₁-C₃)alkylcarbonyl;
      in particular, R¹⁰ and R¹¹, which may be identical or different, represent a hydrogen atom or a (C₁-C₆)alkyl group substituted with one or more hydroxyl, cyano or (C₁-C₃)alkylcarbonyl groups such as methyl, ethyl, butyl, isobutyl, cyanoethyl, methylcarbonylethyl, or hydroxyethyl;
m represents an integer between 1 and 18 inclusive; particularly an integer between 2 and 16 inclusive; preferentially an integer between 3 and 10; more preferentially an integer between 4 and 6;
M′ represents an anionic counterion derived from salts of organic or mineral acids preferably originating from Y;
   it being understood that when the molecule comprises an alkoxide group, then M′ and Q⁺ may be absent to ensure the electrical neutrality of said molecule.
   According to one embodiment of the invention, the dye(s) are of formula (XII) with:

| R¹ | R² | R³ | R⁴ | R⁵ | R⁶ | R⁷ | R⁸ | R⁹ | m |
|---|---|---|---|---|---|---|---|---|---|
| H | H | H | H | H | H | OCH₃ | OCH₃ | OCH₃ | 2 |
| H | H | H | H | OH | OCH₃ | H | H | H | 2 |
| H | H | H | H | H | H | H | H | H | 2 |
| H | H | H | H | H | OCH₃ | OCH₃ | H | H | 2 |
| H | H | H | H | OH | H | OH | H | H | 6 |
| H | H | H | H | OCH₃ | H | OCH₃ | OCH₃ | H | 6 |
| H | H | H | H | H | H | OH | H | H | 6 |
| H | H | H | H | OCH₃ | H | H | H | F | 2 |
| H | H | H | H | H | H | C(O)—OH | H | H | 2 |
| H | H | H | H | H | H | Isopropyl | H | H | 2 |
| H | H | H | H | H | H | N(CH₂CH₂C(O)CH₃)₂ | H | H | 2 |
| H | H | H | H | OH | H | OCH₃ | H | H | 2 |
| H | H | H | H | H | H | OH | H | H | 2 |
| H | H | H | H | H | OCH₃ | OH | OH | H | 2 |
| H | H | H | H | H | CH₃ | OCH₂Ph | CH₃ | H | 2 |
| H | H | H | H | H | OCH₃ | OCH₃ | OCH₃ | H | 2 |
| H | H | H | H | H | OCH₃ | OCH₃ | OCH₃ | H | 6 |
| H | H | H | H | H | H | N(CH₃)₂ | H | H | 6 |
| H | H | H | H | H | H | OCH₃ | OCH₃ | OCH₃ | 6 |
| H | H | H | H | H | H | Phenyl | H | H | 6 |
| H | H | H | H | H | OCH₃ | OCH₃ | H | H | 6 |
| H | H | H | H | H | H | C(O)—OH | H | H | 6 |
| H | H | H | H | H | H | N(n-Butyl)₂ | H | H | 2 |
| H | H | H | H | H | H | OCH₃ | OCH₃ | H | 3 |
| H | H | H | H | H | H | OCH₃ | OCH₃ | H | 2 |
| H | H | H | H | H | H | OCH₃ | OCH₃ | H | 5 |
| H | H | H | H | H | H | OCH₃ | H | H | 3 |
| H | H | H | H | H | H | N(CH₃)₂ | H | H | 3 |
| H | H | H | H | H | H | H | H | H | 3 |

and also the organic or mineral acid or base salts thereof, the optical isomers, geometrical isomers and tautomers thereof, and the solvates thereof such as hydrates.
According to one embodiment, the dye(s) are of formula (XIII) with:

| R¹ | R² | R³ | R⁴ | R⁵ | R⁶ | R⁷ | R⁸ | R⁹ | m |
|---|---|---|---|---|---|---|---|---|---|
| CH₃ | H | H | H | OH | H | OCH₃ | H | H | 2 |
| CH₃ | H | H | H | H | H | OCH₃ | OCH₂—Ph | H | 2 |
| CH₃ | H | H | H | H | H | H | H | OCH₃ | 2 |
| CH₃ | H | H | H | F | H | H | H | H | 2 |
| CH₃ | H | H | H | H | H | H | OPh | H | 2 |
| CH₃ | H | H | H | H | H | N(CH₂CH₂OAc)₂ | H | H | 2 |
| CH₃ | H | H | H | OCH₃ | H | OCH₃ | OCH₃ | H | 2 |
| CH₃ | H | H | H | H | H | H | CH₃ | H | 2 |
| CH₃ | H | H | H | H | H | OH | H | H | 2 |
| CH₃ | H | H | H | H | OCH₃ | OCH₃ | OCH₃ | H | 2 |
| CH₃ | H | H | H | H | OCH₃ | OH | OH | H | 2 |
| CH₃ | H | H | H | H | H | OCH₃ | OCH₃ | OCH₃ | 2 |
| CH₃ | H | H | H | H | H | H | OCH₃ | OH | 2 |
| CH₃ | H | H | H | H | H | N(n-butyl)₂ | H | H | 2 |
| CH₃ | H | H | H | H | OCH₃ | OCH₃ | H | H | 2 |
| CH₃ | H | H | H | H | H | H | H | H | 2 |
| CH₃ | H | H | H | H | H | i-propyl | H | H | 2 |
| H | H | H | H | H | H | OH | H | H | 6 |
| H | H | H | H | H | OCH₃ | OCH₃ | OCH₃ | H | 6 |
| H | H | H | H | OH | H | OCH₃ | H | H | 2 |
| H | H | H | H | OCH₃ | H | H | OCH₃ | OCH₃ | 2 |
| H | H | H | H | H | H | H | H | Br | 2 |
| H | H | H | H | H | H | OH | H | H | 2 |
| H | H | H | H | H | H | N(CH₃)₂ | H | H | 6 |
| H | H | H | H | OCH₃ | OCH₃ | OCH₃ | H | H | 6 |
| H | H | H | H | OH | OCH₃ | H | H | H | 6 |
| H | H | H | H | H | OCH₃ | OCH₃ | H | H | 6 |
| H | H | H | H | H | OCH₃ | OCH₃ | H | H | 2 |
| H | H | H | H | H | H | C(O)—OH | H | H | 6 |
| H | H | H | H | H | H | C(O)—OH | H | H | 2 |
| H | H | H | H | H | H | i-propyl | H | H | 2 |
| H | H | H | H | H | H | N(CH₃)CH₂CH₂CN | H | H | 2 |
| H | H | H | H | H | H | OCH₃ | OCH₂Ph | H | 2 |
| H | H | H | H | H | H | H | OPh | H | 2 |
| H | H | H | H | H | H | N(CH₂CH₂C(O)CH₃)₂ | H | H | 2 |
| H | H | H | H | OH | H | OCH₃ | H | H | 6 |
| H | H | H | H | H | OCH₃ | OCH₃ | OCH₃ | H | 2 |
| H | H | H | H | OCH₃ | H | OCH₃ | OCH₃ | H | 6 |
| H | H | H | H | H | H | H | CH₃ | H | 2 |
| H | H | H | H | H | H | N(CH₃)CH₂CH₂OH | H | H | 2 |
| CH₃ | H | H | H | H | H | N(CH₃)₂ | H | H | 2 |

and also the organic or mineral acid or base salts thereof, the geometrical isomers and tautomers thereof, and the solvates thereof such as hydrates.

More particularly, the fluorescent dye (b) of the invention are chosen from those of formulae (XII′) and (XIII′) below:

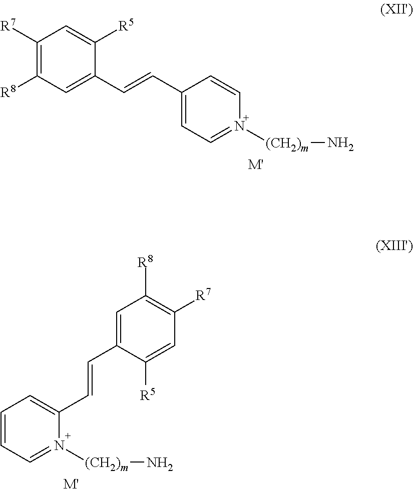

in which formulae (XII) and (XIII) R⁵, R⁷, R⁸ and m are as defined previously for (X) and (XI), in particular:
R⁵ and R⁸, which may be identical or different, represent a hydrogen atom or a (C₁-C₄)alkoxy group such as methoxy;
R⁷ represents a (C₁-C₄)alkoxy group or NR¹⁰R¹¹ with R¹⁰ and R¹¹, which may be identical or different, representing a) a hydrogen atom, or b) a (C₁-C₈)alkyl group optionally substituted with one or more groups chosen from i) cyano, ii) (C₁-C₃)alkoxy, iii) hydroxyl, and iv) (C₁-C₃)alkylcarbonyl; in particular, R¹⁰ and R¹¹, which may be identical or different, represent a hydrogen atom or a (C₁-C₆)alkyl group optionally substituted with one or more groups from the following: hydroxyl, cyano or (C₁-C₃)alkylcarbonyl such as methyl, ethyl, butyl, isobutyl, cyanoethyl, methylcarbonylethyl or hydroxyethyl; preferably, R¹⁰ and R¹¹, which may be identical or different, represent a (C₁-C₆)alkyl group optionally substituted with one or more hydroxyl groups such as hydroxyethyl;
m represents an integer between 1 and 18 inclusive; particularly an integer between 2 and 16 inclusive; preferentially an integer between 3 and 10; more preferentially an integer between 4 and 6; and
M′ represents an anionic counterion, derived from a salt of an organic or mineral acid, or from an organic or mineral base that ensures the electrical neutrality of the molecule.
   According to one embodiment, the fluorescent dye(s) (b) of the invention are of formula (XII′) or (XIII′) with:

| R⁵ | R⁷ | R⁸ | m | | R⁵ | R⁷ | R⁸ | m |
|---|---|---|---|---|---|---|---|---|
| H | N(CH₂CH₂OH)₂ | H | 2 | and | OCH₃ | OCH₃ | OCH₃ | 2 |
| H | N(CH₂CH₂OH)₂ | H | 3 | | OCH₃ | OCH₃ | OCH₃ | 3 |
| H | N(CH₂CH₂OH)₂ | H | 4 | | OCH₃ | OCH₃ | OCH₃ | 3 |
| H | N(CH₂CH₂OH)₂ | H | 5 | | OCH₃ | OCH₃ | OCH₃ | 4 |
| H | N(CH₂CH₂OH)₂ | H | 6 | | OCH₃ | OCH₃ | OCH₃ | 5 |
| H | N(CH₂CH₂OH)₂ | H | 8 | | OCH₃ | OCH₃ | OCH₃ | 8 |
| H | N(CH₂CH₂OH)₂ | H | 10 | | OCH₃ | OCH₃ | OCH₃ | 10 |
| H | N(CH₂CH₂OH)₂ | H | 12 | | OCH₃ | OCH₃ | OCH₃ | 12 |
| H | N(CH₂CH₂OH)₂ | H | 14 | | OCH₃ | OCH₃ | OCH₃ | 14 |
| H | N(CH₂CH₂OH)₂ | H | 16 | | OCH₃ | OCH₃ | OCH₃ | 16 |

and also the organic or mineral acid or base salts thereof, the geometrical isomers and tautomers thereof, and the solvates thereof such as hydrates.

More particularly, the fluorescent dyes (b) of the invention are chosen from those of formula (V), (VIII) or (IX) as defined previously in which G represents a hydrogen atom.

More particularly, the fluorescent dyes (b) of the invention are chosen from those of formulae (XIV) and (XV) below:

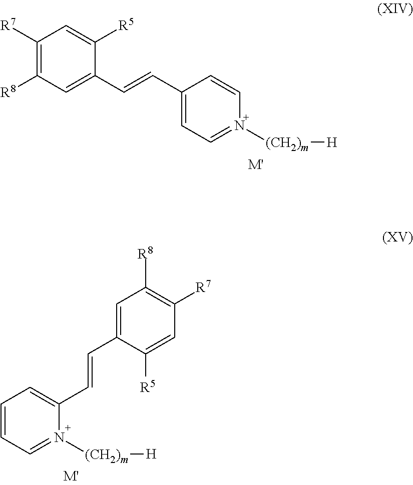

in which formulae (XIV) and (XV) R⁵, R⁷, R⁸ and m are as defined previously for (X) and (XI), in particular:
R⁵ and R⁸, which may be identical or different, represent a hydrogen atom or a (C₁-C₄)alkoxy group such as methoxy, preferably, R⁵ and R⁸ represent a hydrogen atom;
R⁷ represents a (C₁-C₄)alkoxy group or NR¹⁰R¹¹ with R¹⁰ and R¹¹, which may be identical or different, representing a) a hydrogen atom, or b) a (C₁-C₈)alkyl group optionally substituted with one or more groups chosen from i) hydroxyl, ii) R—Z—C(X)—Y— with X, Y and Z representing an oxygen or sulfur atom or N(R′), or alternatively X and/or Z represent a bond, R and R′, which may be identical or different, represent a hydrogen atom or a (C₁-C₆)alkyl group, preferably, X represents an oxygen atom, iii) sulfonic SO₃H, iv) sulfonate SO₃⁻, Q⁺, v) carboxylate C(O)O⁻, Q⁺ with Q⁺ representing a cationic counterion such as an alkali metal or alkaline-earth metal; in particular, R⁷ represents a group NR¹⁰R¹¹ with R¹⁰ and R¹¹, which may be identical or different, representing a) a hydrogen atom, or b) a (C₁-C₆)alkyl group optionally substituted with one or more groups chosen from i) hydroxyl, ii) carboxyl, iii) carboxylate, iv) sulfonic, and v) sulfonate, more particularly chosen from identical or different groups representing a) a hydrogen atom, or b) a (C₁-C₆)alkyl group optionally substituted with one or more groups chosen from i) hydroxyl, ii) carboxyl, and iii) carboxylate;
m represents an integer between 1 and 18 inclusive; particularly an integer between 1 and 6 inclusive; preferentially an integer between 1 and 4; more preferentially an integer between 1 and 2; and
M′ represents an anionic counterion, derived from a salt of an organic or mineral acid, or from an organic or mineral base that ensures the electrical neutrality of the molecule;
it being understood that when the molecule comprises a sulfonate or carboxylate group, then M′ and Q⁺ may be absent to ensure the electrical neutrality of said molecule.

According to one embodiment, the fluorescent dye(s) (b) of the invention are chosen from the following compounds, the geometrical isomers thereof, the tautomers thereof, the solvates thereof, and mixtures thereof:

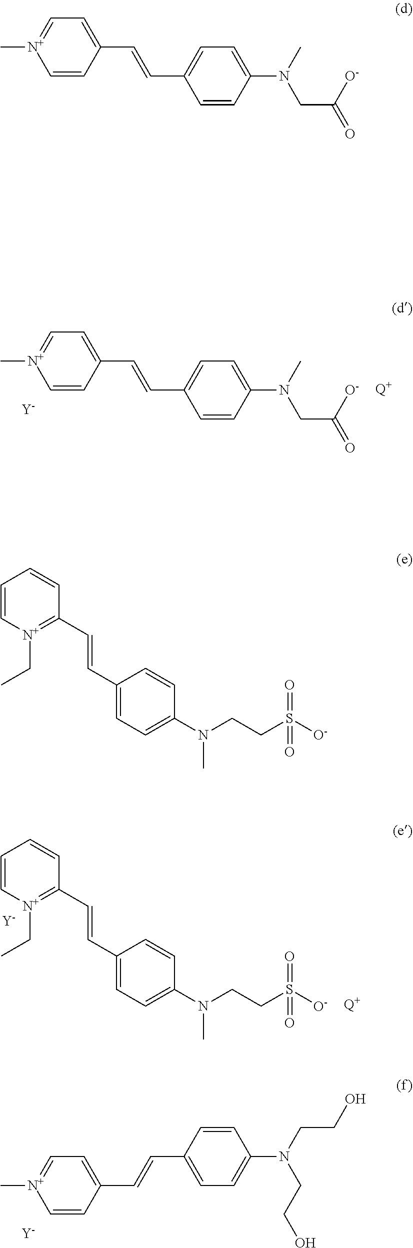

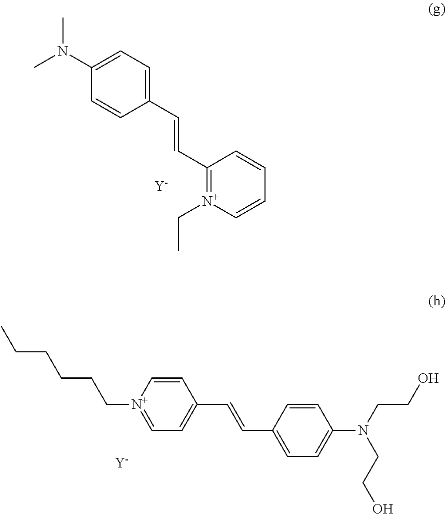

with Y— representing an anionic counterion and Q⁺ representing a cationic counterion as defined previously.

### The Oxidizing Agents

The process for dyeing keratin fibres according to the present invention may optionally also comprise the application to said keratin fibres of one or more oxidizing agents.

Preferably, the oxidizing agent(s) are chosen from chemical oxidizing agents.

The term “chemical oxidizing agent” means an oxidizing agent other than atmospheric oxygen.

More particularly, the chemical oxidizing agent(s) are chosen from hydrogen peroxide, hydrogen peroxide-generating systems, urea peroxide, alkali metal bromates or ferricyanides, peroxygenated salts, for instance persulfates, perborates, peracids and precursors thereof and percarbonates of alkali metals or alkaline-earth metals, and mixtures thereof.

Preferably, the chemical oxidizing agent(s) are chosen from hydrogen peroxide and hydrogen peroxide-generating systems.

According to a preferred embodiment, the hydrogen peroxide-generating system(s) are chosen from urea peroxide, polymeric complexes that can release hydrogen peroxide, chosen from polyvinylpyrrolidone/H₂O₂; oxidases; perborates; and percarbonates.

Preferably, the chemical oxidizing agent is hydrogen peroxide, and more preferentially aqueous hydrogen peroxide solution.

The chemical oxidizing agent(s) are advantageously applied in the form of an aqueous solution of which the content of chemical oxidizing agents is preferably between 0.05% and 5% by weight and more preferentially between 0.1% and 2% by weight, relative to the total weight of the aqueous solution.

According to a preferred embodiment of the invention, the dyeing process does not use any oxidizing agent. According to a preferred embodiment of the invention, the cosmetic composition comprising ingredients (a) and (b) does not comprise any oxidizing agent.

### The Reducing Agents

The process for dyeing keratin fibres according to the present invention may optionally also comprise the application to said keratin fibres of one or more reducing agents. The reducing agent(s) that are useful in the present invention are advantageously chosen from the compounds of formula (XVI) below, and also the addition salts thereof, and mixtures thereof:

H(X)_{q}(R₁₀)ₜ (XVI)

in which compound of formula (XVI):
X represents P, S or SO₂,
q represents an integer equal to 0 or 1,
t represents an integer equal to 1 or 2, and
R represents a linear or branched, saturated or unsaturated C₁ to C₂₀ alkyl radical, optionally interrupted with a heteroatom, and/or optionally substituted with one or more radicals chosen from hydroxyl, halo, amine, carboxyl, ((C₁-C₃₀)alkoxy)carbonyl, amido, ((C₁-C₃₀)alkyl)aminocarbonyl, (C₁-C₃₀)acyl)amino, mono- or dialkylamino, and mono- or dihydroxylamino radicals.
   Preferably, the reducing agent(s) are chosen from thioglycolic acid, thiolactic acid, glyceryl monothioglycolate, cysteamine, N-acetylcysteamine, N-propionylcysteamine, cysteine, N-acetylcysteine, thiomalic acid, pantetheine, 2,3-dimercaptosuccinic acid, N-(mercaptoalkyl)-ω-hydroxyalkylamides, N-mono- or N,N-dialkylmercapto-4-butyramides, aminomercaptoalkylamides, N-(mercaptoalkyl)succinamic acid and N-(mercaptoalkyl)succinimide derivatives, alkylamino mercaptoalkylamides, the azeotropic mixture of 2-hydroxypropyl thioglyconate and of (2-hydroxy-1-methyl)ethyl thioglycolate, mercaptoalkylaminoamides, N-mercaptoalkylalkanediamides and formamidinesulfinic acid derivatives, salts thereof, and mixtures thereof.
   The chemical reducing agent(s) are advantageously applied in the form of an aqueous solution of which the content of chemical reducing agents is preferably between 0.01% and 10% by weight and more preferentially between 0.1% and 5% by weight, relative to the total weight of the aqueous solution.

According to a preferred embodiment of the invention, the dyeing process does not use any reducing agent. According to a preferred embodiment of the invention, the cosmetic composition comprising ingredients (a) and (b) does not comprise any reducing agent.

### The Cosmetic Medium and the Solvents

The anthraquinone compound(s) of formula (I) or (II) as defined previously, and (b) the fluorescent dye(s) as defined previously, and also, when they are present, the oxidizing agent(s) and/or the reducing agent(s), may be dissolved beforehand before being applied to the keratin fibres.

In other words, the ingredients used in the dyeing process of the present invention may be present in one or more compositions.

The composition(s) comprising the ingredients according to the present invention are cosmetic compositions, i.e. they are preferably aqueous. Besides water, they may comprise one or more organic solvents, or mixtures thereof.

Examples of organic solvents that may be mentioned include linear or branched C₂ to C₄ alkanols, such as ethanol and isopropanol; glycerol; polyols and polyol ethers, for instance 2-butoxyethanol, propylene glycol, hexylene glycol, dipropylene glycol, propylene glycol monomethyl ether, diethylene glycol monomethyl ether and monoethyl ether, and also aromatic alcohols or ethers, for instance benzyl alcohol or phenoxyethanol, and mixtures thereof.

### The pH:

The pH of the composition(s) used in the dyeing process of the invention and of the composition of the invention comprising ingredients (a) and (b) is particularly between 2 and 12 approximately and preferably between 3 and 11 approximately. It may be adjusted to the desired value by means of acidifying or alkaline agents usually used in the dyeing of keratin fibres, or alternatively using standard buffer systems.

The pH of the composition which comprises (a) and (b) and that of the composition(s) used in the dyeing process of the invention is preferentially between 6 and 11 inclusive, particularly between 7 and 10 and more particularly between 7.5 and 9.5, such as between 9 and 9.5.

Among the acidifying agents, mineral and organic acids as defined previously, mention may be made, by way of example, of mineral or organic acids, for instance hydrochloric acid, orthophosphoric acid or sulfuric acid, carboxylic acids, for instance acetic acid, tartaric acid, citric acid and lactic acid, and sulfonic acids.

Among the alkaline agents that may be mentioned, for example, are aqueous ammonia, alkali metal carbonates, alkanolamines such as monoethanolamines, diethanolamines and triethanolamines, and other alkaline agents as defined previously.

### Forms of the Composition:

The composition(s) comprising the dye(s) of formula (I) as defined previously, and the fluorescent dye(s) as defined previously, may be in various presentation forms, such as in the form of liquids, lotions, creams or gels or in any other form that is suitable for dyeing keratin fibres. It may also be packaged under pressure in an aerosol container in the presence of a propellant or in a non-aerosol container and may form a foam.

### Additives

When the ingredients used in the dyeing process according to the present invention are present in one or more composition(s), said composition(s) may also optionally comprise one or more additives, different from the ingredients of the invention and among which mention may be made of fatty substances, cationic, anionic, nonionic, amphoteric or zwitterionic surfactants, cationic, anionic, nonionic or amphoteric polymers or mixtures thereof, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, especially polymeric thickeners, opacifiers or nacreous agents, antioxidants, hydroxy acids, fragrances, preserving agents, pigments and ceramides.

Needless to say, a person skilled in the art will take care to select this or these optional additional compound(s) such that the advantageous properties intrinsically associated with the composition(s) in accordance with the invention are not, or are not substantially, adversely affected by the envisaged addition(s).

The above additives may generally be present in an amount, for each of them, of between 0 and 20% by weight relative to the total weight of the composition comprising them.

### The Dyeing Process

The process for dyeing keratin fibres according to the present invention comprises the application to said keratin fibres of the following ingredients:
(a) one or more anthraquinone dyes of formulae (I) and/or (II) as defined previously, and
(b) one or more fluorescent dyes as defined previously,
   it being understood that the anthraquinone dye(s) of formulae (I) and/or (II) (ingredients (a)) and the fluorescent dye(s) (ingredients (b)) are applied to said keratin fibres together or sequentially.

In other words, the dyeing process according to the present invention may be performed in one or more steps.

According to a particularly preferred embodiment, the anthraquinone dye(s) of formulae (I) and/or (II) and the fluorescent dye(s) (b), as defined previously, are applied together (or jointly), i.e. simultaneously, to the keratin fibres. According to this embodiment, the dyeing process is performed in one step. One variant of the dyeing process of the invention involves applying a cosmetic composition according to the invention which comprises (a) the anthraquinone compound(s) of formulae (I) and/or (II) and (b) the fluorescent dye(s) as defined previously to the keratin fibres.

According to another particularly preferred embodiment, the anthraquinone dye(s) of formulae (I) and/or (II) as defined previously and the fluorescent dye(s) as defined previously are applied sequentially, i.e. successively. According to this other embodiment, the dyeing process is performed in at least two steps.

According to a first embodiment in at least two steps, the fluorescent dye(s) as defined previously are applied to the keratin fibres subsequently to the anthraquinone dye(s) of formulae (I) and/or (II) as defined previously. In other words, the fluorescent dye(s) as defined previously are applied after the anthraquinone dye(s) of formulae (I) and/or (II) as defined previously.

According to this first embodiment, the process for dyeing keratin fibres comprises the following steps:
a first step of applying to said keratin fibres a cosmetic composition comprising one or more anthraquinone dye(s) of formulae (I) and/or (II) as defined previously, followed by
a second step of applying to said keratin fibres a cosmetic composition which comprises one or more fluorescent dyes as defined previously.

According to a preferred embodiment in at least two steps, the anthraquinone dye(s) of formulae (I) and/or (II) as defined previously are applied to the keratin fibres subsequently to the fluorescent dye(s). In other words, the anthraquinone dye(s) of formulae (I) and/or (II) are applied after the fluorescent direct dye(s).

According to this preferred embodiment, the process for dyeing keratin fibres comprises the following steps:
a first step of applying to said keratin fibres a cosmetic composition comprising one or more fluorescent dyes as defined previously, followed by
a second step of applying to said keratin fibres a cosmetic composition comprising one or more anthraquinone dye(s) of formulae (I) and/or (II) as defined previously.

Preferably, ingredients (a) and (b) are applied to the keratin fibres in a bath ratio that may range from 0.1 to 10 and more particularly from 0.2 to 8. For the purposes of the present invention, the term “bath ratio” means the ratio between the total weight of ingredient (a) or (b) and the total weight of keratin fibres to be treated.

When the dyeing process is performed in one step, ingredients (a) and (b) are advantageously left to stand on the keratin fibres for a time ranging from 1 to 90 minutes and more preferentially for a time ranging from 5 to 60 minutes.

When the dyeing process is performed in at least two steps, each of the ingredients (a) and (b) may be advantageously left to stand on the keratin fibres for a time ranging from 1 to 60 minutes and more preferentially for a time ranging from 5 to 45 minutes.

On conclusion of the dyeing process according to the invention, in one or at least two steps, the keratin fibres are advantageously rinsed with water. They may optionally be washed with a shampoo, followed by rinsing with water, before being dried or left to dry.

When the dyeing process is performed in at least two steps, the keratin fibres are advantageously rinsed with water between each step. In other words, the dyeing process may comprise an intermediate rinsing step between the application of the first ingredient and the application of the second ingredient. During this intermediate rinsing step, the keratin fibres may optionally be washed with a shampoo, followed by rinsing with water, before being dried or left to dry.

The dyeing process according to the present invention may be performed at room temperature (25° C.) or with heating.

When they are present, the reducing agent(s) may be applied separately or together with one of the ingredients (a) or (b). Preferably, when they are present, the reducing agent(s) are applied together with the ingredient (b).

When they are present, the oxidizing agent(s) may be applied separately or together with one of the ingredients (a) or (b). Preferably, when they are present, the oxidizing agent(s) are applied after application of ingredients (a) and (b).

According to a particular embodiment, the process for dyeing keratin fibres according to the present invention comprises the following successive steps:
a first step of applying to said keratin fibres a cosmetic composition comprising (a) one or more anthraquinone dye(s) of formulae (I) and/or (II) as defined previously, followed by
a second step of applying to said keratin fibres a cosmetic composition comprising: (b) one or more fluorescent dyes as defined previously, and (c) one or more reducing agents as defined previously.

According to a particular embodiment of the dyeing process of the invention, no step of said process involves an oxidizing agent.

According to an advantageous embodiment of the dyeing process of the invention, no step of said process involves a reducing agent.

The dyeing process according to the present invention may be applied to wet or dry, preferably dry, keratin fibres.

### The Multi-Compartment Device

The present invention also relates to a multi-compartment device comprising a first compartment comprising one or more anthraquinone dyes of formula (I) and/or (II) as defined previously, and a second compartment comprising one or more fluorescent dyes as defined previously.

According to a particular embodiment, the “keratin fibres” are human keratin fibres and more particularly the hair.

The examples that follow serve to illustrate the invention without, however, being limiting in nature.

### EXAMPLES

### 1. Application Tests Performed:

The tests were performed:
at a molar concentration of 5×10⁻³ mol % of each dye (Example 1)
at a molar concentration of 5×10⁻³ mol % of anthraquinone dye and of 1.66×10⁻³ mol % of fluorescent dye (Example 2)
The solutions are adjusted to pH 9-9.5 with aqueous ammonia solution.
   The compounds tested on a 0.5 g lock of hair containing 90% white hairs are the following:
   The violet-blue dyes, of the anthraquinone disulfide series or not, evaluated in the context of the invention are the following:

| Dye (a) | Structure of violet-blue dyes |
|---|---|
| Dye 1 | 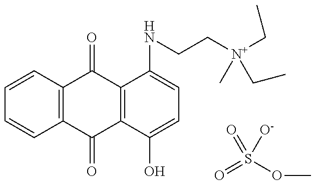 |
| Dye 2 | 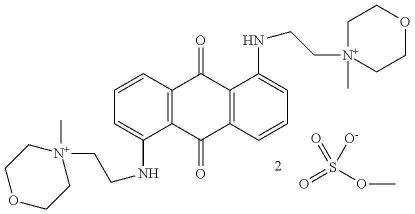 |
| Dye 3 | 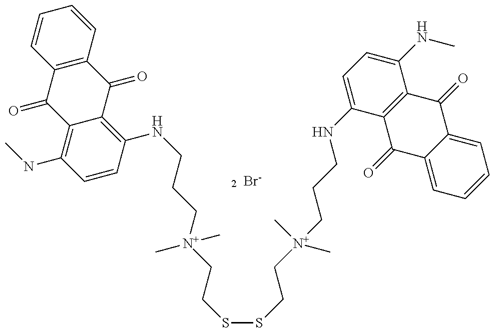 [SMILES: CNc1ccc(NCCC[N+](C)(C)CCSSCC[N+](C)(C)CCCNc2ccc(NC)c3c2C(=O)c2ccccc2C3=O)c2c1C(=O)c1ccccc1C2=O ; [Br-]] |

The fluorescent dyes of the styrylpyridinium series evaluated in the context of the invention are the following:

| Dye (b) | Structure of the fluorescent dyes |
|---|---|
| Dye a | 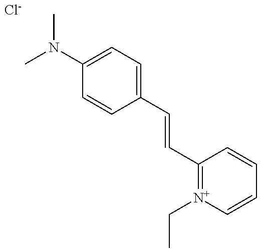 [SMILES: [Cl-] ; CC[n+]1ccccc1/C=C/c1ccc(N(C)C)cc1] |
| Dye b | 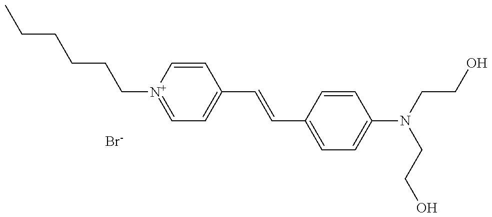 [SMILES: CCCCCC[n+]1ccc(/C=C/c2ccc(N(CCO)CCO)cc2)cc1 ; [Br-]] |

The combinations of dyeing compounds were prepared on a 0.5 g lock of natural hair containing 90% white hairs as a mixture (NW90) according to 3 application modes:

In Reducing Medium:
“blue direct dye” then “fluorescent dye” (application sense 1—AS1)

In Non-Reducing Medium:
“fluorescent dye” then “blue direct dye” (application sense 2—AS2)
“blue direct dye” then “fluorescent dye” (application sense 3—AS3)
“blue direct dye” and “fluorescent dye” as a mixture before application (application sense 4—AS4).
   For application sense 1—AS1, the products were applied as follows:
   Application condition for the blue direct dye/fluorescent dye:
   The amount of a) dye of formula (I) or (II) according to the invention [blue] and of b) fluorescent dyes=5×10⁻³ mol % in water
9 ml of this mixture+1 ml of Dulcia DV2 (bath ratio=5)
20 minutes at room temperature (RT=25° C.)+rinsing+H₂O₂(0.48 g %) 5 minutes at RT
rinsing+1 shampooing+drying under a hood for 10 minutes per g of hair
   For application senses 2 (AS2), 3 (AS3) and 4 (AS4), the products were applied as follows:
   Application condition for the blue direct dye of formula (I) or (II): 5×10−3 mol % in water, 30 minutes at RT+rinsing+1 shampooing+drying under a hood for 10 minutes per g of hair.
   Application condition for the fluorescent dye: 5×10⁻³ mol % in water, 20 minutes at RT+rinsing+1 shampooing+drying under a hood for 10 minutes per g of hair
   Application condition for the mixture “blue direct dye of formula (I) or (II)+fluorescent dye”: 5×10⁻³ mol % of each of the dyes in water, 45 minutes at RT+rinsing+1 shampooing+drying under a hood for 10 minutes per g of hair

### Example 1

The following hair dyeing results were obtained:

| Blue dye | Fluorescent dye | Application sense | Colour obtained |
|---|---|---|---|
| Dye 1 | Dye a | AS2 | Brown with a |
| | | | coppery glint |
| Dye 1 | Dye a | AS3 | Brown with a |
| | | | coppery glint |
| Dye 1 | Dye a | AS4 | Brown with a |
| | | | coppery glint |
| Dye 1 | Dye b | AS1 | Brown with a |
| | | | coppery glint |
| Dye 2 | Dye a | AS1 | Brown with a |
| | | | coppery glint |
| Dye 2 | Dye b | AS1 | Brown with a |
| | | | coppery glint |
| Dye 2 | Dye b | AS2 | Brown with a |
| | | | coppery glint |
| Dye 2 | Dye b | AS3 | Brown with a |
| | | | coppery glint |
| Dye 2 | Dye b | AS4 | Brown with a |
| | | | coppery glint |
| Dye 3 | Dye a | AS1 | Dull brown |
| Dye 3 | Dye b | AS1 | Brown |

It is seen that the process of the invention makes it possible to obtain very aesthetic and powerful brown colours just by combining a blue dye and a fluorescent dye.

### Comparative Data:

### Comparative Experiment

3 series of locks (90% natural with hair—90 NW) have been carried out with an equimolar association of a reductive or not medium vs. comparative prior art EP0852943A1.
The tests has been carried out at a molar concentration of 5×10-3 mol % for each dye. The compositions are adjusted to pH 9-9.5 with an ammonia solution. The compounds tested on locks of 0.5 g of natural white hair (90 NWV) are the following:
The blue disulfide anthraquinone dye, and the hemicyanin dyes have been evaluated in the context of the comparison as follows:

| Dye (a) | chemical structure of Blue-violet dye |
|---|---|
| Dye 3 | 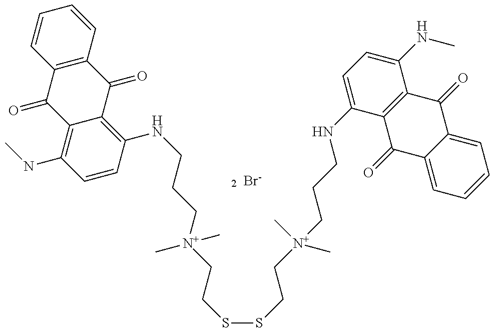 [SMILES: CNc1ccc(NCCC[N+](C)(C)CCSSCC[N+](C)(C)CCCNc2ccc(NC)c3c2C(=O)c2ccccc2C3=O)c2c1C(=O)c1ccccc1C2=O ; [Br-]] |

| Dye (b) | chemical structure of hemicyanin fluorescent dyes |
|---|---|
| Dye a EP0852943A1 Comparative | 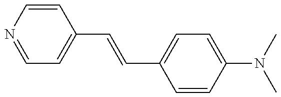 [SMILES: CN(C)c1ccc(/C=C/c2ccncc2)cc1] |
| Dye b Invention | 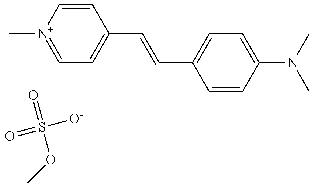 |

### In Reducing Medium:

“blue disulfide direct dye” and “fluorescent dye” (application sense 1—SA1)
   Application condition of the blue disulfide direct dye/fluorescent dye:
   The amount of a) dye 3 and b) fluorescent dyes=5.10-3 mol % in water
9 ml of this mixture+1 ml dulcia DV2
20′/TA+rinse+H₂O₂ (0.48 g %) 5′/TA
rinsing+1 shampoo+drying with headphones 10′/g of hair
In a non-reducing medium:
   “blue disulfide direct dye” and “fluorescent dye” mixed before application (method 2—SA2).
      The method of applying the mixture «blue disulfide direct dye 3+fluorescent dye» 5.10-3 mol % in water, for 45° at room temperature (27° C.)/TA+rinsing+1 shampoo+drying au hair 10′/g of hair

### Results

| Blue direct dye | Fluorescent dye | application sense | Color after treatment |
|---|---|---|---|
| Dye 3 | | AS1 | Blue |
| Dye 3 | | AS2 | Blue |
| | Dye a | AS1 | No coloration |
| | Dye a | AS2 | No coloration |
| | Dye b | AS1 | Red |
| | Dye b | AS2 | Red |
| Dye 3 | Dye a | AS1 | Red |
| Dye 3 | Dye b | AS1 | Brown with shade |
| Dye 3 | Dye a | AS2 | Blue |
| Dye 3 | Dye b | AS2 | Cold brown |

After treating keratin fibers with dyes and shampoo locks have been dried and color on the locks has been measured with spectrocolorimeter Minolta CM2600d (angle 10°, illuminant D65) in the CIEL*a*b* system.
In this system, L* represents intensity of the color, a* represents the green/red color axis and b* the blue/yellow color axis.
The lowest L* measured, the most intensive is the color.

| Blue Dye | Fluorescent dye | Application sense | L* | a* | b* |
|---|---|---|---|---|---|
| Dye 3 | | AS1 | 32.03 | −5.72 | −19 |
| Dye 3 | | AS2 | 35.41 | −7.06 | −14.56 |
| | Dye a | AS1 | 59.86 | 0.54 | 15.83 |
| | Dye a | AS2 | 61.59 | 0.65 | 10.98 |
| | Dye b | AS1 | 28.23 | 38.77 | 25.94 |
| | Dye b | AS2 | 33.27 | 43.76 | 31.17 |
| Dye 3 | Dye a | AS1 | 32.25 | −5.03 | −18.65 |
| Dye 3 | Dye b | AS1 | 19.29 | 10.66 | 8.56 |
| Dye 3 | Dye a | AS2 | 35.82 | −5.89 | −17.44 |
| Dye 3 | Dye b | AS2 | 24.3 | 18.77 | 16.51 |

These results of the table show that the method according to the invention, using only two direct fluorescent dyes b), anthraquinone dye of formula (I) and/or (II) of the invention associated with a cationic fluorescent dye, makes it possible to obtain natural brown colors. This is not the case of the combination of an anthraquinone dye of formula (I) and/or (II) of EP0852943A1 combined with a non-cationic fluorescent dye a) for which the color obtained is blue (non natural color).

1.-27. (canceled)

28. A process for dyeing keratin fibers, comprising applying to the keratin fibers ingredients (a) and (b) below:
(a) at least one anthraquinone dye chosen from the compounds of formulae (I) and/or (II) below, optical isomers thereof, geometrical isomers thereof, tautomers thereof, solvates thereof, or mixtures thereof: 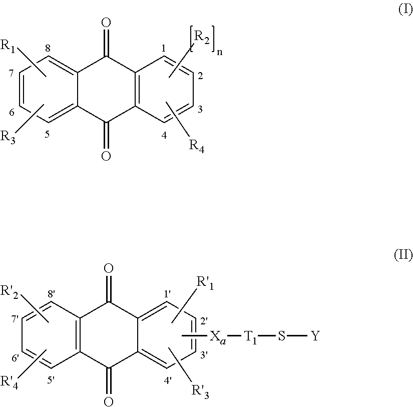
wherein in formula (I) or (II):
Xₐ, which may be identical or different, represents a bond, a heteroatom or a group chosen from an oxygen or sulfur atom, N(Rₐ), CO, SO, SO₂, or combinations thereof, with Rₐ representing a hydrogen atom or a (C₁-C₆)alkyl group optionally substituted with one or more hydroxyl groups;
Y represents: i) a hydrogen atom; ii) an alkali metal; iii) an alkaline-earth metal; iv) an ammonium group: N⁺R^{a}R^{b}R^{g}R^{d} or a phosphonium group: P⁺R^{a}R^{b}R^{g}R^{d} with R^{a}, R^{b}, R^{g} and R^{d}, which may be identical or different, representing a hydrogen atom or a (C₁-C₄)alkyl group; or v) a thiol-function protecting group; or vi) the group (III) below: 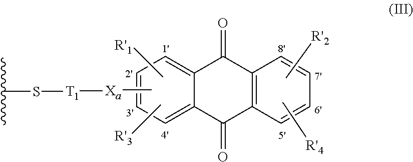
R₁, R₂, R₃, R₄, R′₁, R′₂, R′₃ and R′₄, which may be identical or different, represent an atom or a group chosen from:
hydrogen;
halogen such as bromine and chlorine,
hydroxyl,
C₁-C₄ alkoxy,
hydroxysulfonyl (—SO₃H) or sulfonate (—SO₃⁻, M⁺), with M⁺ representing a cationic counterion;
optionally substituted C₁-C₆ alkyl,
—NR₅R₆ in which R₅ and R₆, which may be identical or different, represent an atom or radical chosen from: i) hydrogen, ii) (C₁-C₄)alkylcarbonyl, iii) arylsulfonyl such as phenylsulfonyl (—SO₂Ph), iv) Het-ALK-C(O)— with Het representing a heterocycloalkyl group which is optionally substituted and ALK represents a (C₁-C₆)alkylene group optionally substituted with one or more hydroxyl or (di)(hydroxy)(C₁-C₄)(alkyl)amino groups; v) optionally substituted aryl, vi) optionally substituted aryl(C₁-C₄)alkyl, viii) optionally substituted C₁-C₂₀ alkyl, optionally interrupted with one or more heteroatoms and/or with one or more groups comprising at least one heteroatom;
a group of formula (a) below:
—N(R₇)—X₁—W₁ (a)
wherein in formula (a):
R₇ represents a hydrogen or a C₁-C₄ alkyl radical,
X₁ represents a divalent radical chosen from C₁-C₂₀ alkylene optionally interrupted with one or more heteroatoms or groups chosen from oxygen, nitrogen, sulfur, CO, SO, SO₂, arylene, or combinations thereof;
W₁ represents a cationic radical chosen from: 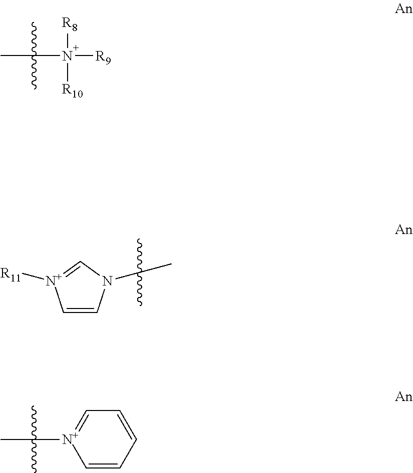
with R₈, R₉, R₁₀ and R₁₁, which may be identical or different, representing a C₁-C₆ alkyl group, a benzyl radical, a C₁-C₆ alkyl sulfonate radical; the radicals R₈ and R₉ optionally form, with the nitrogen atom to which they are attached, a saturated or unsaturated, optionally substituted 5- to 7-membered heterocycle, optionally comprising another non-nitrogen heteroatom;
n is an integer ranging from 1 to 3;
T₁ represents a linear or branched divalent hydrocarbon-based chain comprising from 1 to 20 carbon atoms, optionally interrupted with one or more heteroatoms or groups, or combinations thereof, chosen from oxygen, sulfur, N(R_{b}), C(O), —N⁺(R₈)(R₉)-An, optionally cationic and optionally substituted heteroaryl, An with R₈ and R₉, which may be identical or different, represent a C₁-C₆ alkyl radical; R_{b} representing a hydrogen atom or a (hydroxy)(C₁-C₄)alkyl group; [symbol]
being the part of the bond that is connected to the rest of the molecule; and
(b) at least one cationic (poly)methine fluorescent dye;
wherein:
the anthraquinone dye(s) of formula (I) comprise at least one radical R₁, R₃ or R₄, other than a hydrogen atom, and the anthraquinone dye(s) of formula (II) comprise at least one radical R′₁, R′₂, R′₃ or R′₄ other than a hydrogen atom;
(a) the anthraquinone dye(s) of formula (I) or (II) and (b) the fluorescent dye(s) are applied to said keratin fibers together or sequentially; and
when the compounds of formula (I) or (II) are cationic and comprise a sulfonate group, then M⁺ and An are optionally absent to ensure the electrical neutrality of said molecule.

29. The process according to claim 28, wherein the at least one anthraquinone dye of formulae (I) and/or (II) absorbs light in the blue-violet range.

30. The process according to claim 28, wherein the at least one anthraquinone is chosen from compounds of formula (I) wherein n is equal to 0 or 1.

31. The process according to claim 28, wherein the at least one anthraquinone dye is chosen from compounds of formula (I), wherein R₁ and R₃ represent an atom or group chosen from i) hydroxyl, ii) arylamino or aryl(C₁-C₆)alkylamino with the aryl group representing an optionally substituted aryl group, iii) (di)(hydroxy)(C₁-C₆)(alkyl)amino, iv) (di)(C₁-C₄)(alkyl)amino(C₁-C₆)alkylamino, v) (C₁-C₄)alkylcarbonylamino(C₁-C₆)alkylamino, vi) arylsulfonylamino with the aryl group optionally substituted, vii) (di)halo(C₁-C₄)alkylamino, viii) (di)(hydroxy)(C₁-C₄)alkoxy(C₁-C₄)alkylamino, ix) (di)tri(C₁-C₄)alkylammonium(C₁-C₆)alkylamino, x) heterocycloalkyl(C₁-C₆)alkylamino, heterocycloalkyl(C₁-C₄)alkylcarbonylamino, heterocycloalkyl(C₁-C₄)alkylamino(C₁-C₄)alkylcarbonylamino or heterocycloalkyl(C₁-C₄)alkylcarbonylamino(C₁-C₆)alkylamino, wherein the heterocycloalkyl is optionally cationic, xi) halo(C₁-C₄)alkylcarbonylamino(C₁-C₆)alkylamino, xii) hydroxysulfonyl, xiii) halo, xiv) heteroaryl(C₁-C₆)alkylamino, wherein the heteroaryl is optionally cationic and/or substituted, xv) heteroaryl(C₁-C₄)alkylcarbonylamino(C₁-C₆)alkylamino, wherein the heteroaryl is optionally cationic and/or substituted, xvi) R′R″N— with R′ and R″, which may be identical or different, representing a (halo)(C₁-C₄)alkylcarbonylamino(C₁-C₆)alkyl group, a (halo)(C₁-C₄)alkylaminocarbonyl (C₁-C₆)alkyl group, a (halo)(C₁-C₄)alkylcarbonyl group, a carboxy(C₁-C₆)alkyl group, the alkyl group possibly being substituted with one or more amino or hydroxyl groups, a (poly)hydroxy(C₁-C₆)alkyl group, xvii) (C₁-C₁₆)alkylaminocarbonylamino(C₁-C₆)alkylamino, xviii) formylamino(C₁-C₆)alkylamino, xix) (hydroxy)(C₁-C₆)alkylamino(C₁-C₆)alkylamino, xix) hydroxysulfonyl(C₁-C₆)alkylamino(C₁-C₆)alkylamino, xx) sulfonato(C₁-C₆)alkyl(di(C₁-C₄)alkyl)ammonium(C₁-C₆)alkylamino, xxi) hydroxysulfonyl(C₁-C₆)alkoxy(C₁-C₆)alkylamino, xxi) heteroaryl(C₁-C₄)alkylcarbonylamino, wherein the heteroaryl is optionally cationic and/or substituted, xxii) heterocycloalkyl(C₁-C₄)alkylcarbonylamino, wherein the heterocycloalkyl is optionally cationic and/or substituted, xxiii) (di)(C₁-C₄)(alkyl)amino(C₁-C₆)alkylamino, wherein the (C₁-C₆)alkyl group is optionally substituted with one or more hydroxyl groups, xxiv) heterocycloalkylamino(C₁-C₆)alkylamino or heterocycloalkylamino, wherein the heterocycloalkyl is optionally cationic and/or substituted, xxv) heteroarylalkylamino(C₁-C₆)alkylamino or heteroarylalkylamino, wherein the heteroaryl is optionally cationic and/or substituted, xxvi) tri(C₁-C₆)alkylammonium(C₁-C₆)alkylamino, xxvii) (C₁-C₄)alkoxycarbonyl(C₁-C₆)alkyl(di(C₁-C₄)alkyl)ammonium(C₁-C₆)alkylamino, xxviii) carboxylato(C₁-C₆)alkyl(di(C₁-C₄)alkyl)ammonium(C₁-C₆)alkylamino, xxix) carboxy(C₁-C₆)alkylamino(C₁-C₆)alkylamino, xxx) aryl(C₁-C₄)alkyl(di(C₁-C₄)alkyl)ammonium(C₁-C₆)alkylamino, xxxi) sulfonic SO₃H or sulfonate SO₃⁻, M⁺ with M⁺ representing a cationic counterion, xxxii) (C₁-C₆)alkyl, xxxiii) hydroxysulfonyl(C₁-C₄)amino, xxxiv) phenylsulfonylamino.

32. The process according to claim 28, wherein the at least one anthraquinone dye is chosen from dyes of formula (I), wherein R₂ and R₄, which may be identical or different, represent a hydrogen atom or a group chosen from:
hydroxyl,
(di)(C₁-C₄)(alkyl)amino,
(C₁-C₄)alkoxy, heterocycloalkyl(C₁-C₆)alkylamino, wherein the heterocycloalkyl is optionally cationic and/or substituted,
arylamino or aryl(C₁-C₆)alkylamino wherein the aryl group is an optionally substituted aryl group,
aryl(C₁-C₄)alkyl(di(C₁-C₄)alkyl)ammonium(C₁-C₆)alkylamino,
(di)(C₁-C₄)(alkyl)amino(C₁-C₆)alkylamino, or
hydroxy(C₁-C₄)(alkyl)amino(C₁-C₆)alkylamino.

33. The process according to claim 28, wherein the at least one anthraquinone dye is chosen from dyes of formula (I), and R₂ and R₄ represent a hydrogen atom.

34. The process according to claim 28, wherein the at least one anthraquinone dye of formulae (I) and/or (II) is chosen from dyes of formula (I) with R₂ and R₃ representing a hydrogen atom, and R₁ and R₄ are chosen from:
halogen,
(di)(C₁-C₄)(alkyl)amino,
(C₁-C₄)alkylcarbonylamino,
heterocycloalkyl(C₁-C₆)alkylamino, heterocycloalkyl(C₁-C₄)alkylcarbonylamino or heterocycloalkyl(C₁-C₄)alkylamino(C₁-C₄)alkylcarbonylamino, wherein the heterocycloalkyl is optionally cationic and/or substituted,
tri(C₁-C₄)alkylammonium(C₁-C₆)alkylamino,
arylamino or aryl(C₁-C₆)alkylamino wherein the aryl group si an optionally substituted aryl group,
(di)(C₁-C₄)(alkyl)amino(C₁-C₆)alkylamino, or
(C₁-C₄)alkylcarbonylamino(C₁-C₆)alkylamino.

35. The process according to claim 28 wherein the anthraquinone dye(s) of formulae (I) and/or (II) are dyes of formula (I) with R₃ and R₄ representing a hydrogen atom, and R₁ and R₂ are in positions 8 and 1, respectively, and R₁ and R₂ are chosen from:
(di)(hydroxy)(C₁-C₄)(alkyl)amino,
(di)(hydroxy)(C₁-C₄)(alkyl)amino(C₁-C₆)alkylamino,
tri(C₁-C₆)alkylammonium(C₁-C₆)alkylammonium,
tri(C₁-C₆)alkylammonium(C₁-C₆)alkylamino,
heterocycloalkyl(C₁-C₆)alkylamino, said heterocycloalkyl possibly being optionally cationic and/or substituted especially with one or more (C₁-C₄) alkyl or benzyl groups and
arylamino or aryl(C₁-C₆)alkylamino with the aryl group representing an optionally substituted aryl group, in particular a phenyl group optionally substituted with one or more groups chosen from (C₁-C₄)alkyl, (di)(hydroxy)(C₁-C₄)(alkyl)amino, (C₁-C₄)alkoxy, tri(C₁-C₄)alkylammonium.

36. The process according to claim 28, wherein the at least one anthraquinone dye is chosen from dyes of formula (I) with R₁ and R₃ representing a hydrogen atom and n is equal to 2, and R₂ and R₄ are chosen from:
(C₁-C₆)alkyl,
(di)(hydroxy)(C₁-C₄)(alkyl)amino,
SO₃H or SO₃⁻M⁺,
(di)(hydroxy)(C₁-C₄)(alkyl)amino(C₁-C₆)alkylamino,
tri(C₁-C₄)alkylammonium(C₁-C₆)alkylamino, and
heterocycloalkyl(C₁-C₆)alkylamino, wherein the heterocycloalkyl si optionally cationic and/or substituted, or
arylamino or aryl(C₁-C₆)alkylamino with the aryl group representing an optionally substituted aryl group.

37. The process according to claim 28, wherein the at least one anthraquinone dye is chosen from dyes of formula (II′): 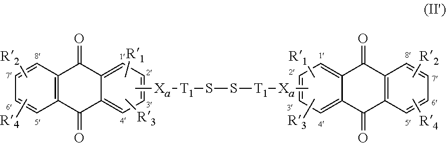
wherein Xₐ represents an N(Rₐ), R′₂ and R′₄ represent a hydrogen atom and; T₁ represents a saturated linear divalent hydrocarbon-based chain comprising from 1 to 20 carbon atoms, optionally interrupted with one or more groups chosen from N(R_{b}), C(O), —N⁺(R₈)(R₉)-An, cationic heteroaryl, An, or combinations thereof, with R₈ and R₉, which may be identical or different, representing a C₁-C₆ alkyl radical; R_{b} representing a hydrogen atom or a (C₁-C₄)alkyl group.

38. The process according to claim 37, wherein the at least one anthraquinone dye is chosen from the dyes of formula (II″):

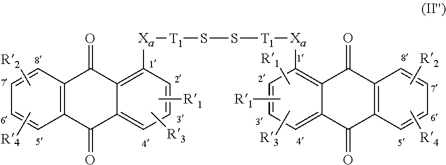

39. The process according to claim 28, wherein the at least one anthraquinone dye of formulae (I) and/or (II) is chosen from the following compounds, optical isomers thereof, geometrical isomers thereof, tautomers thereof, solvates thereof, or mixtures thereof: 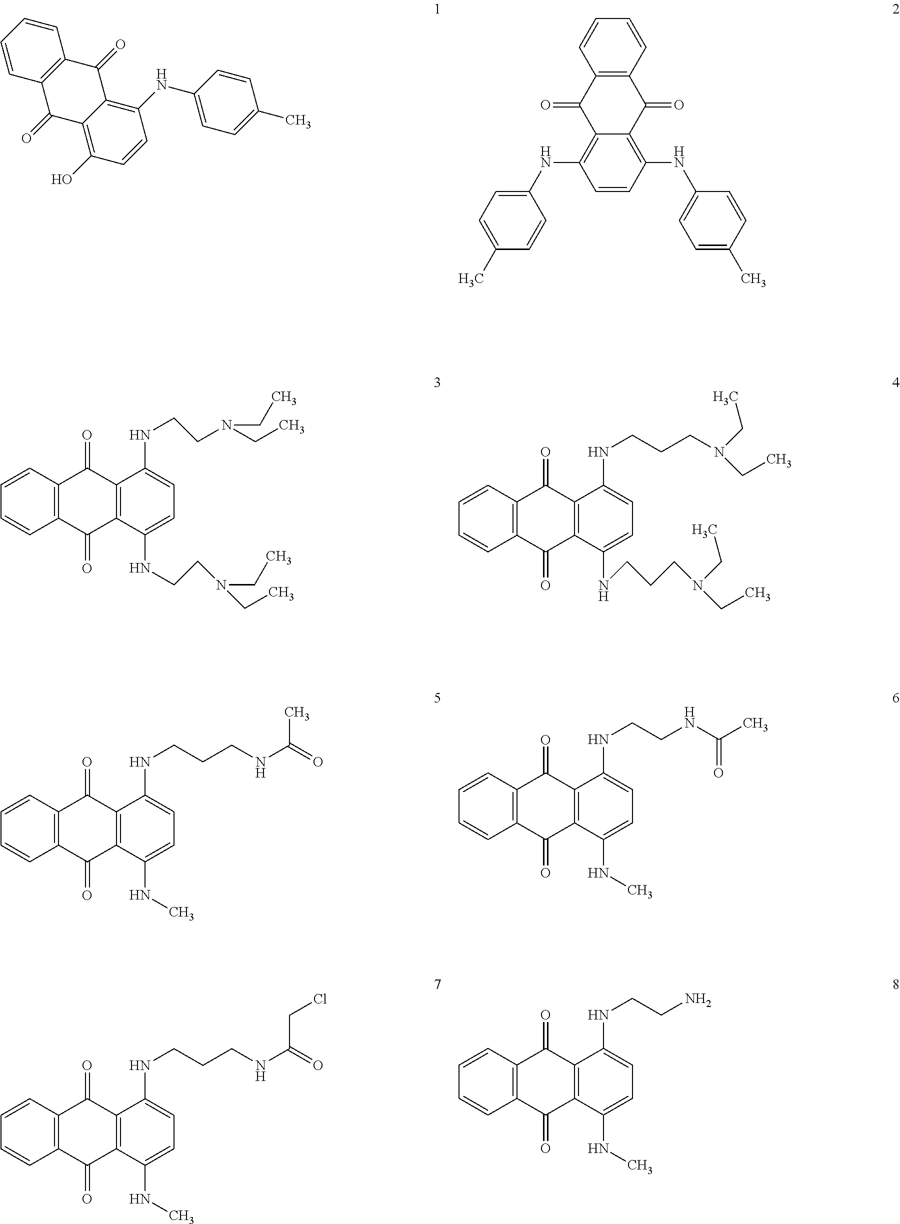 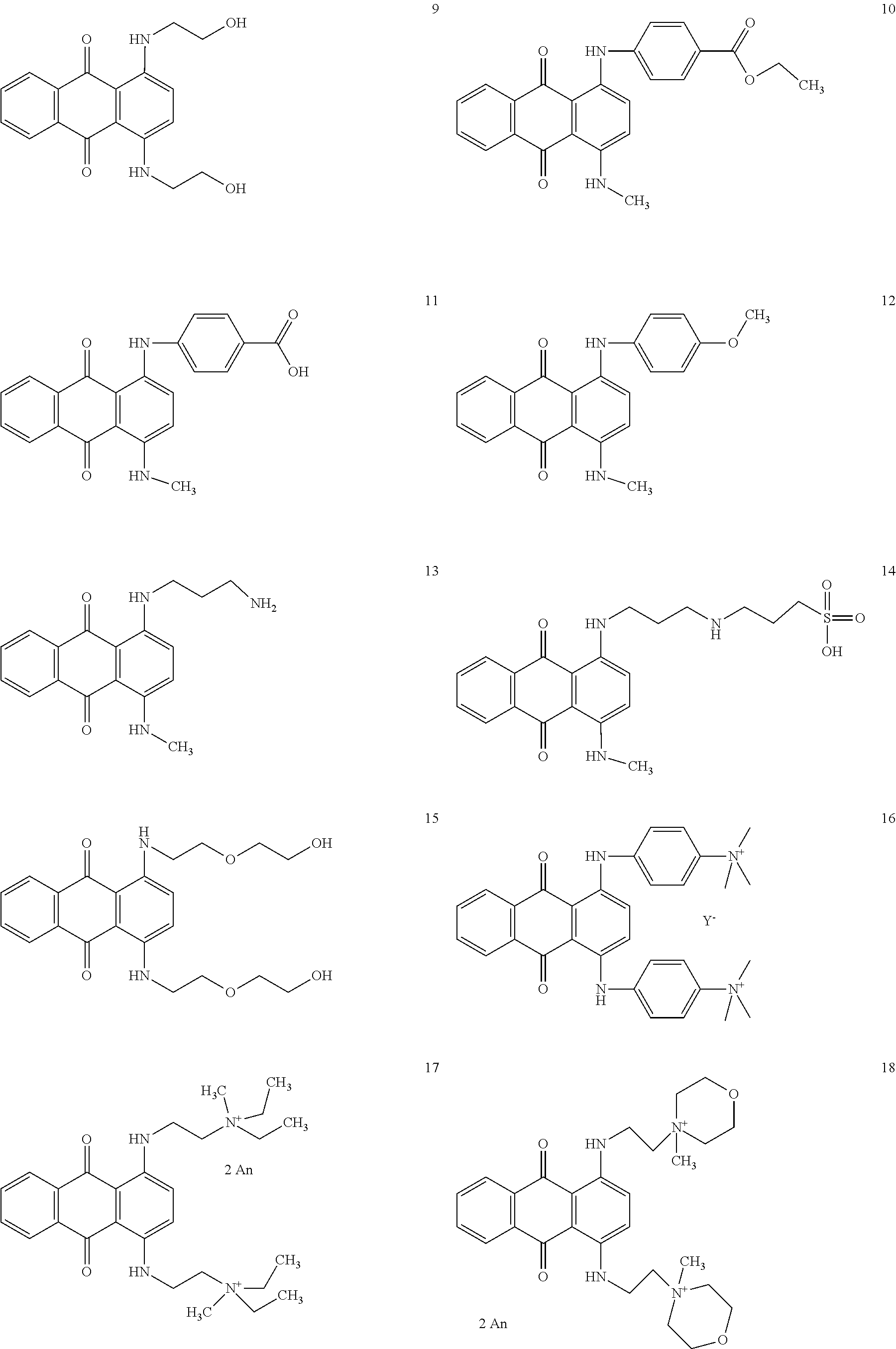 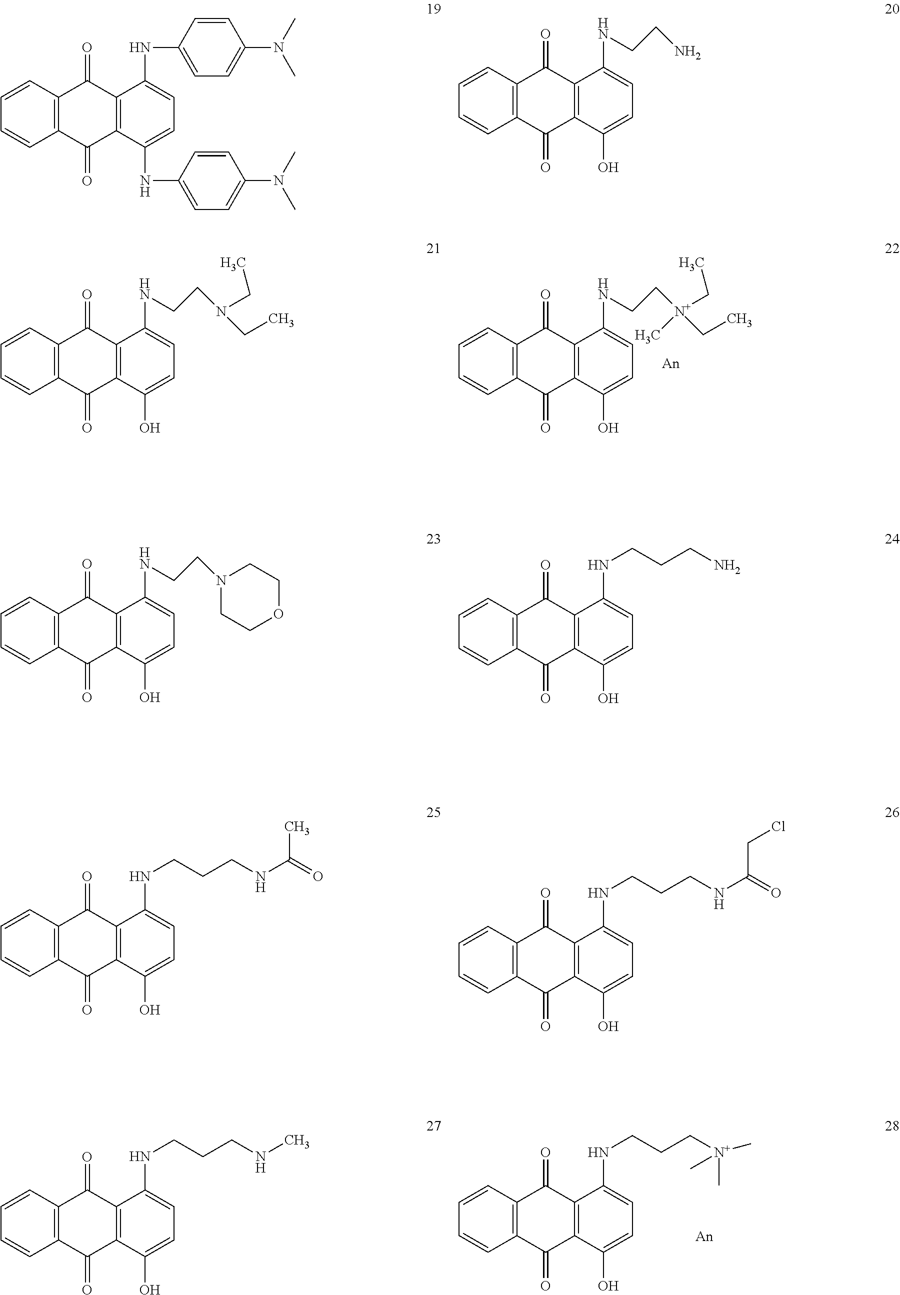 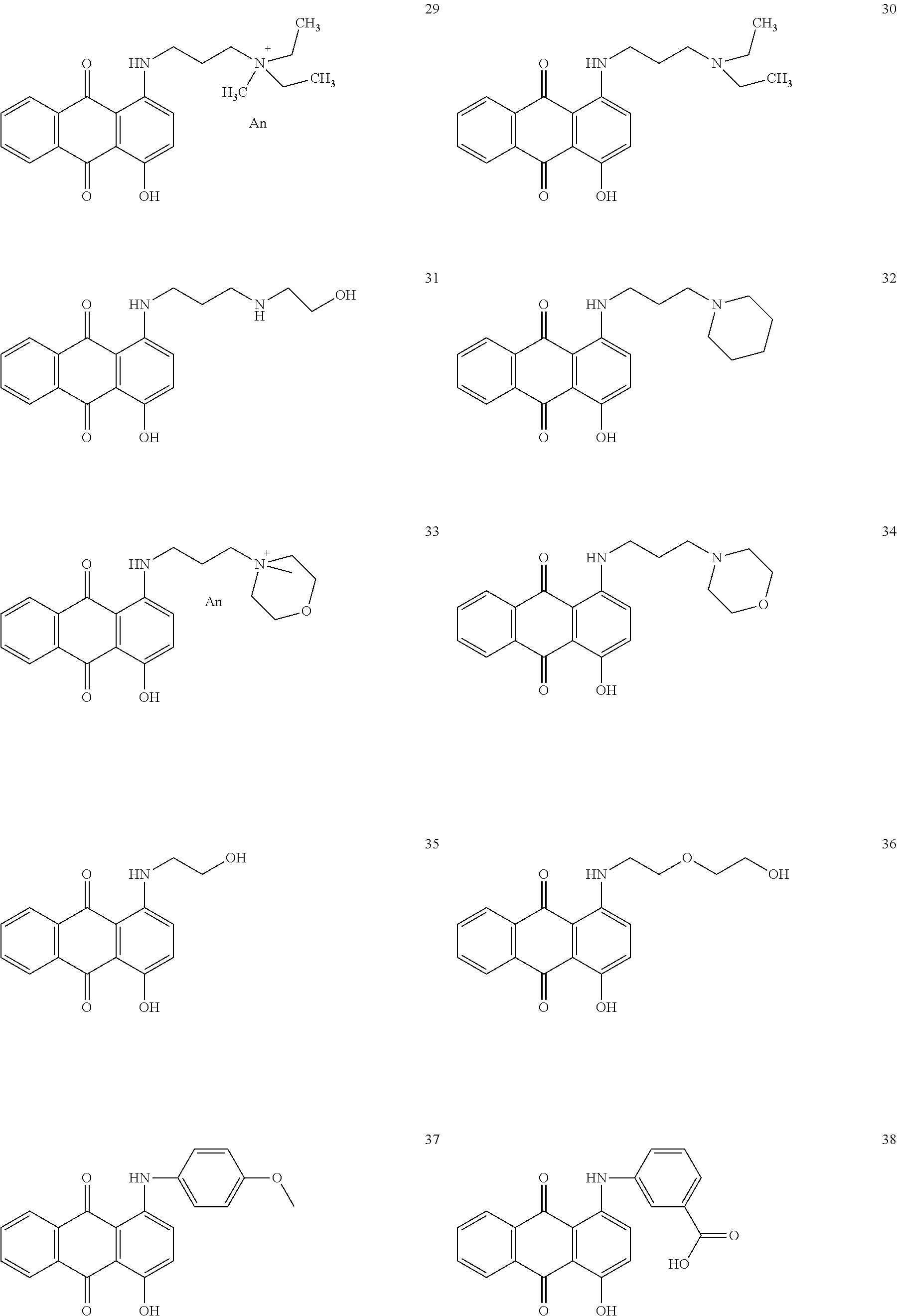 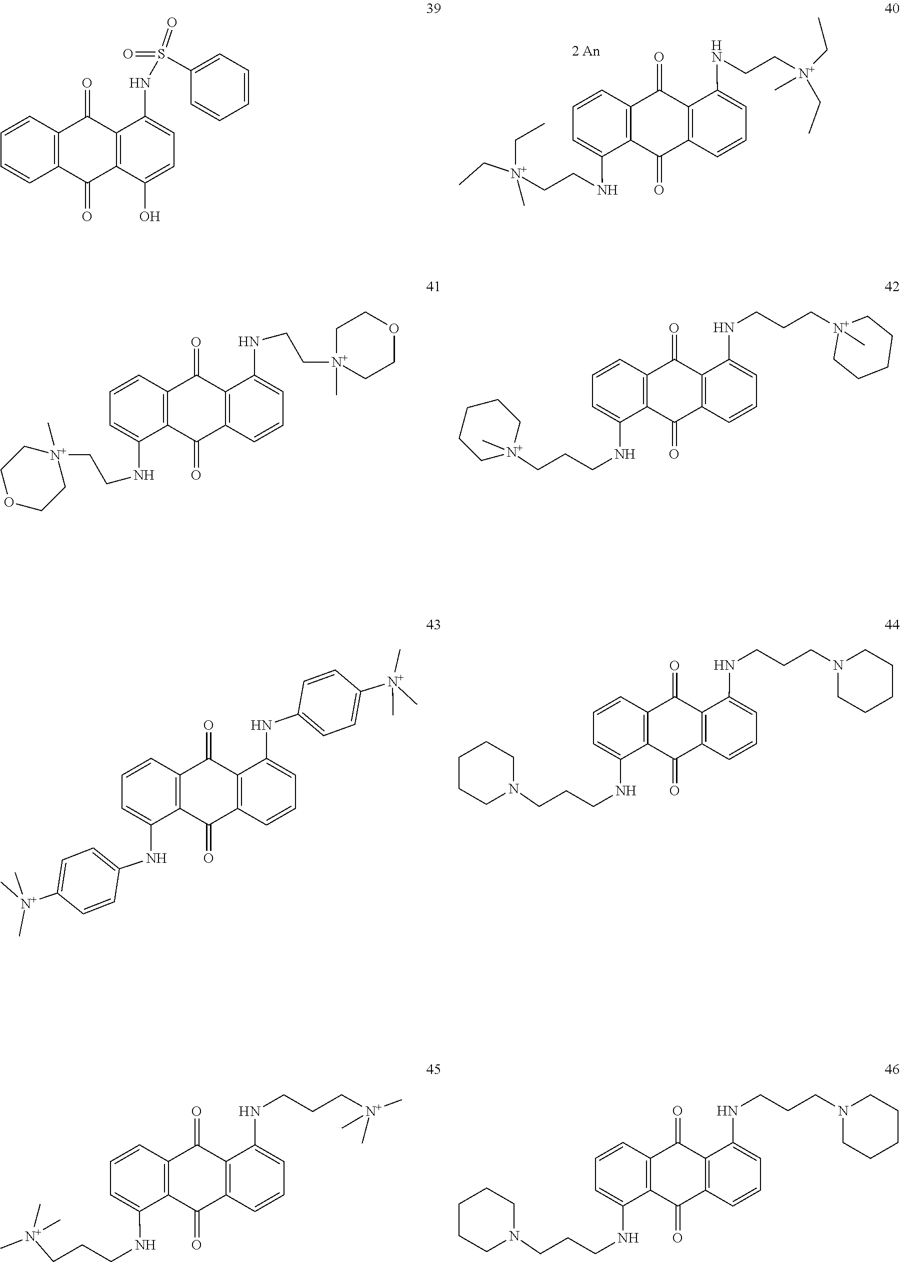 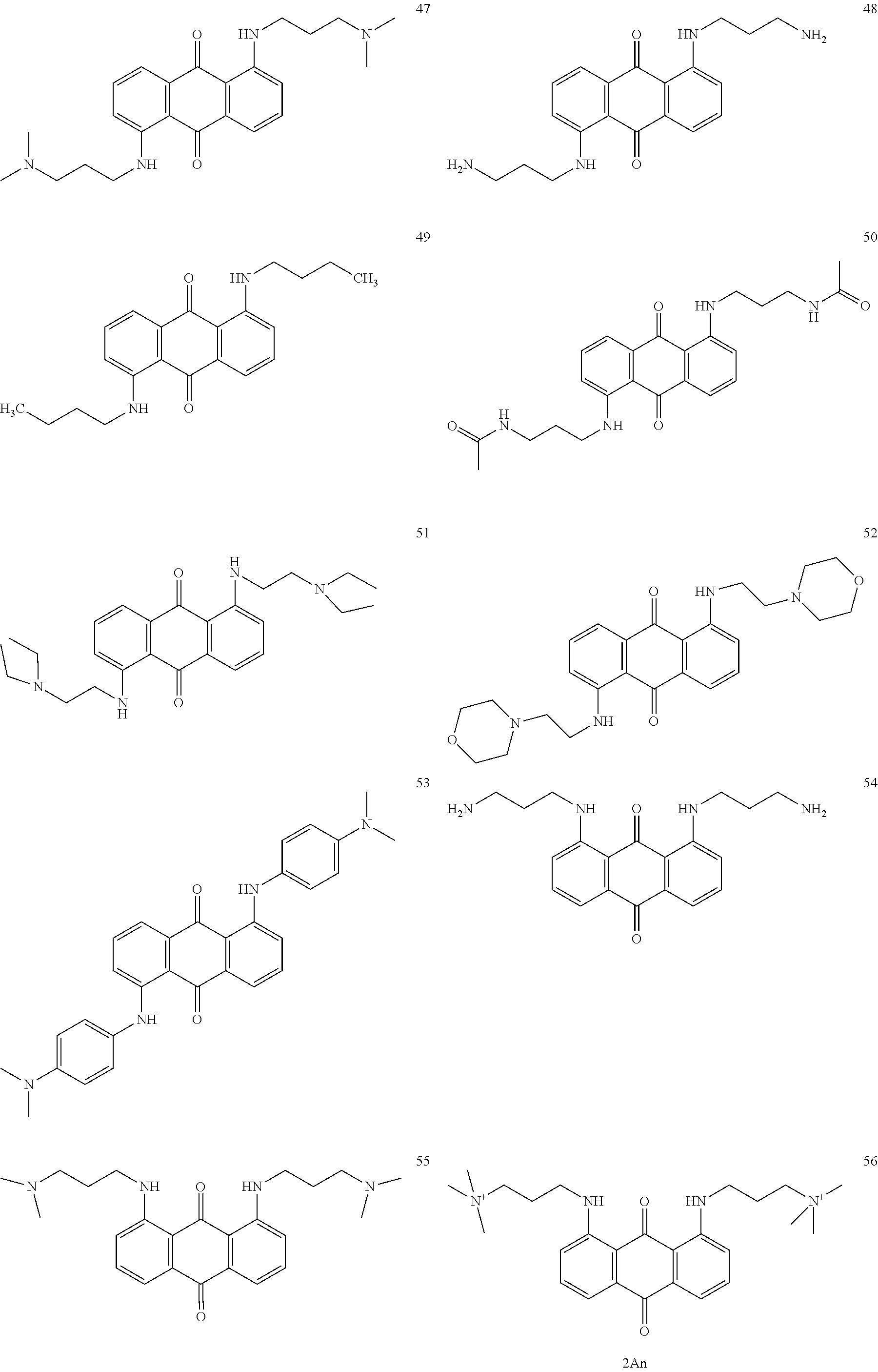 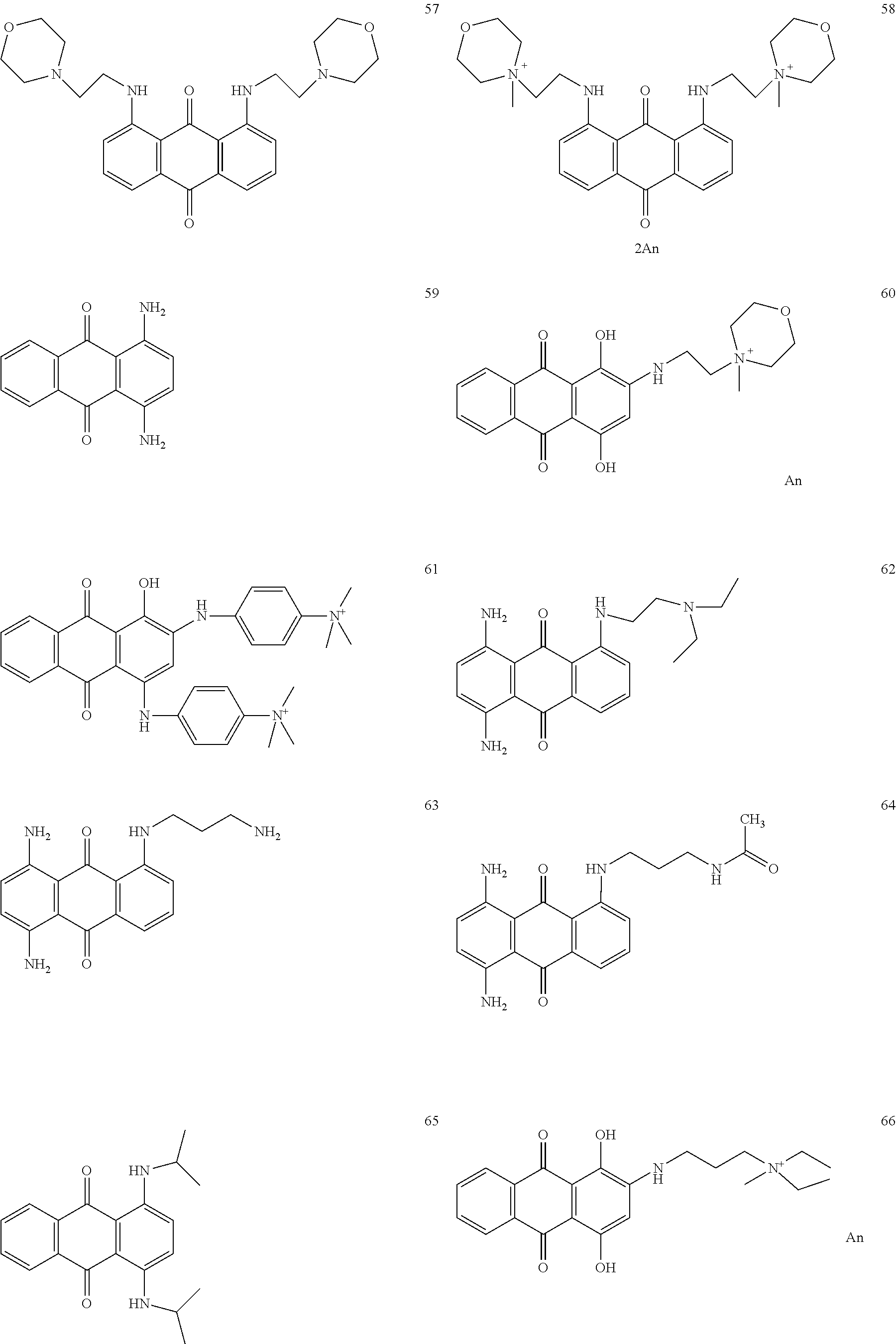 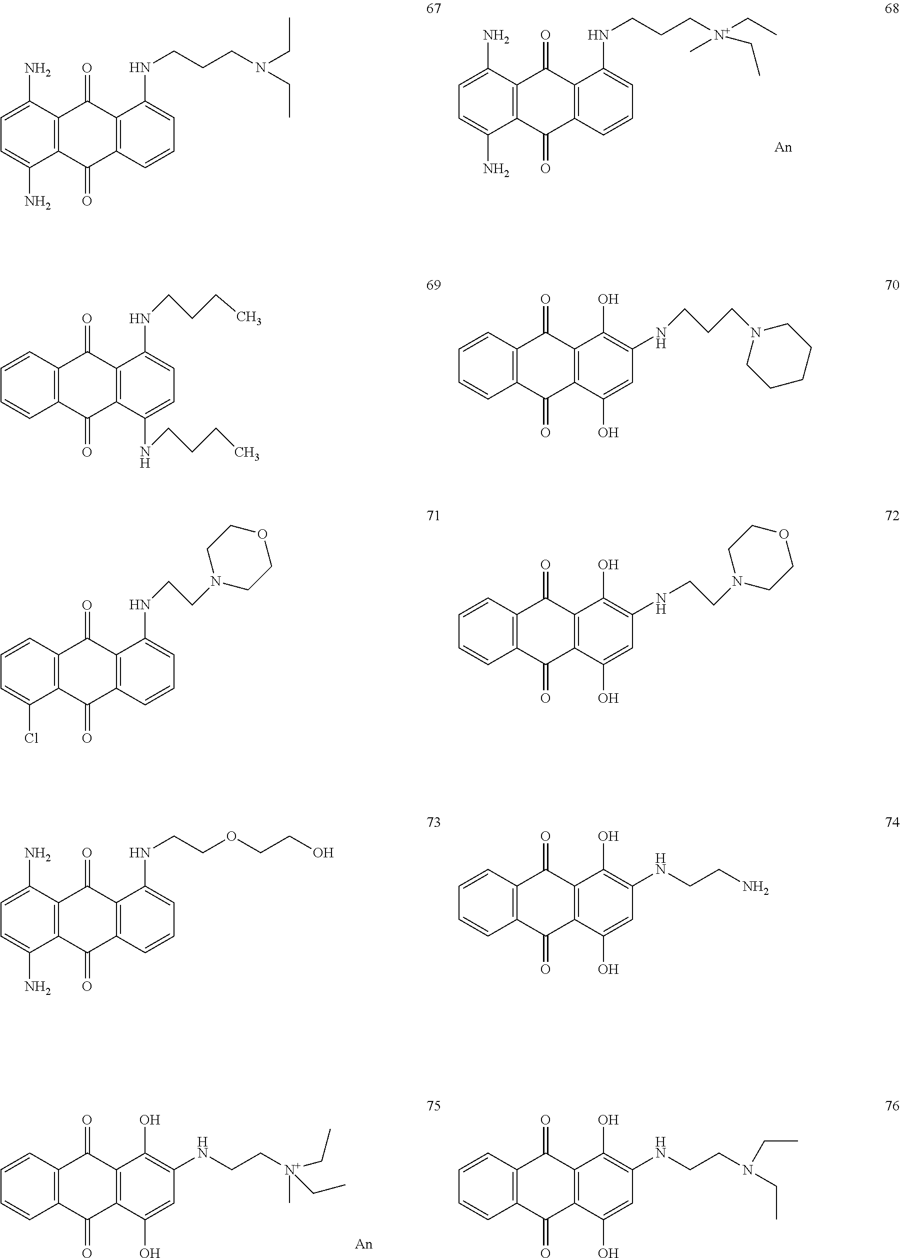 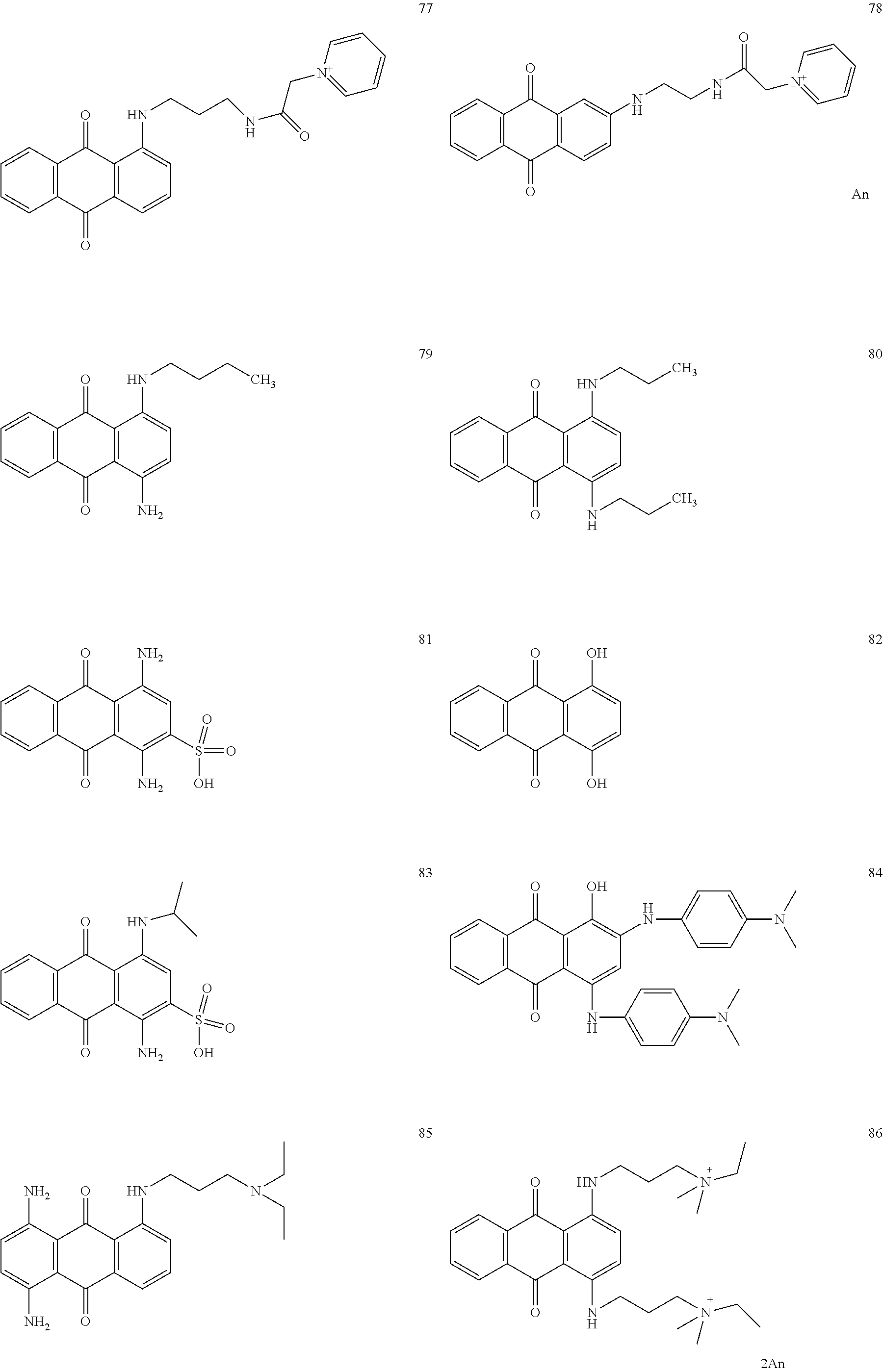 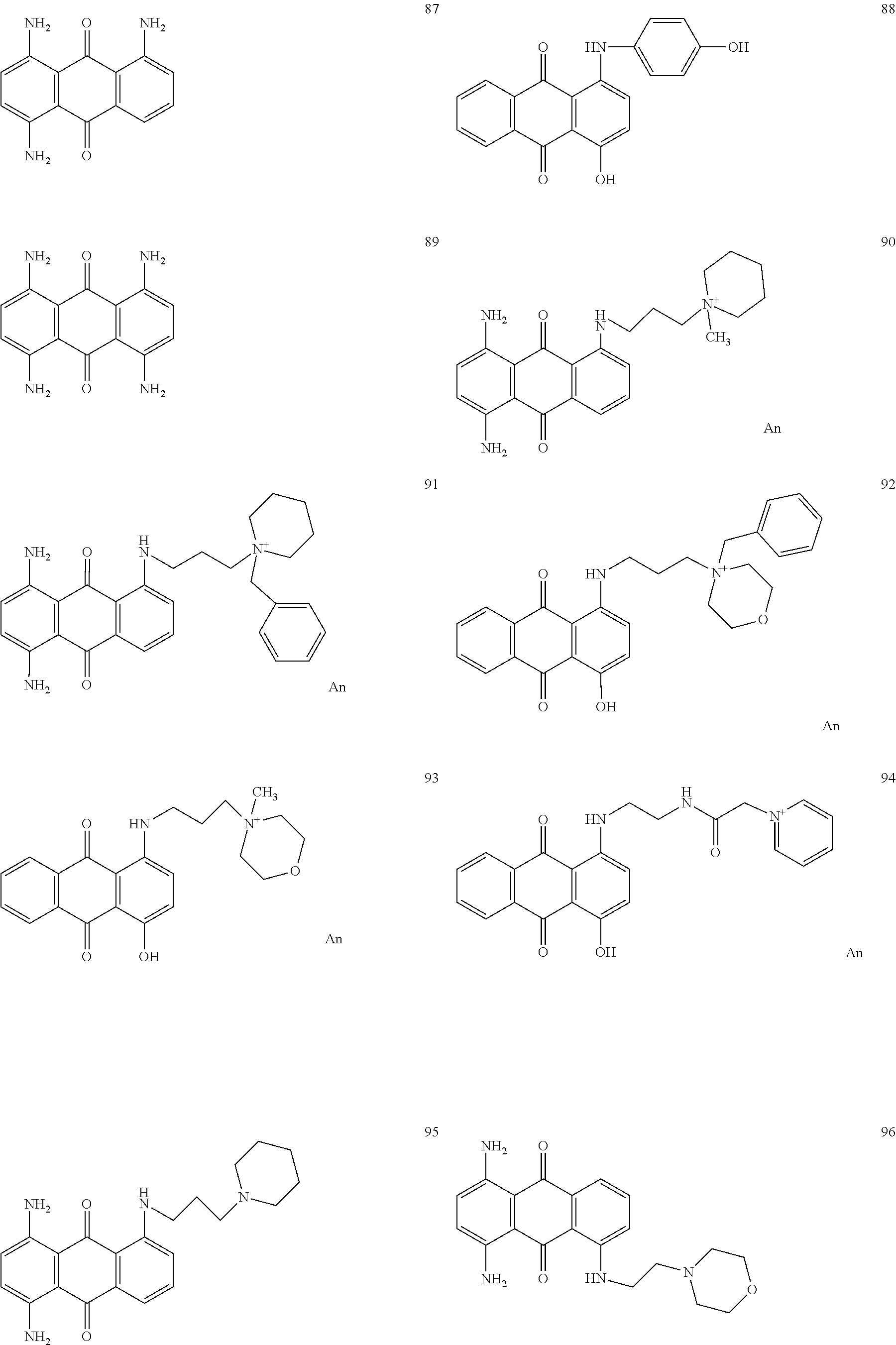 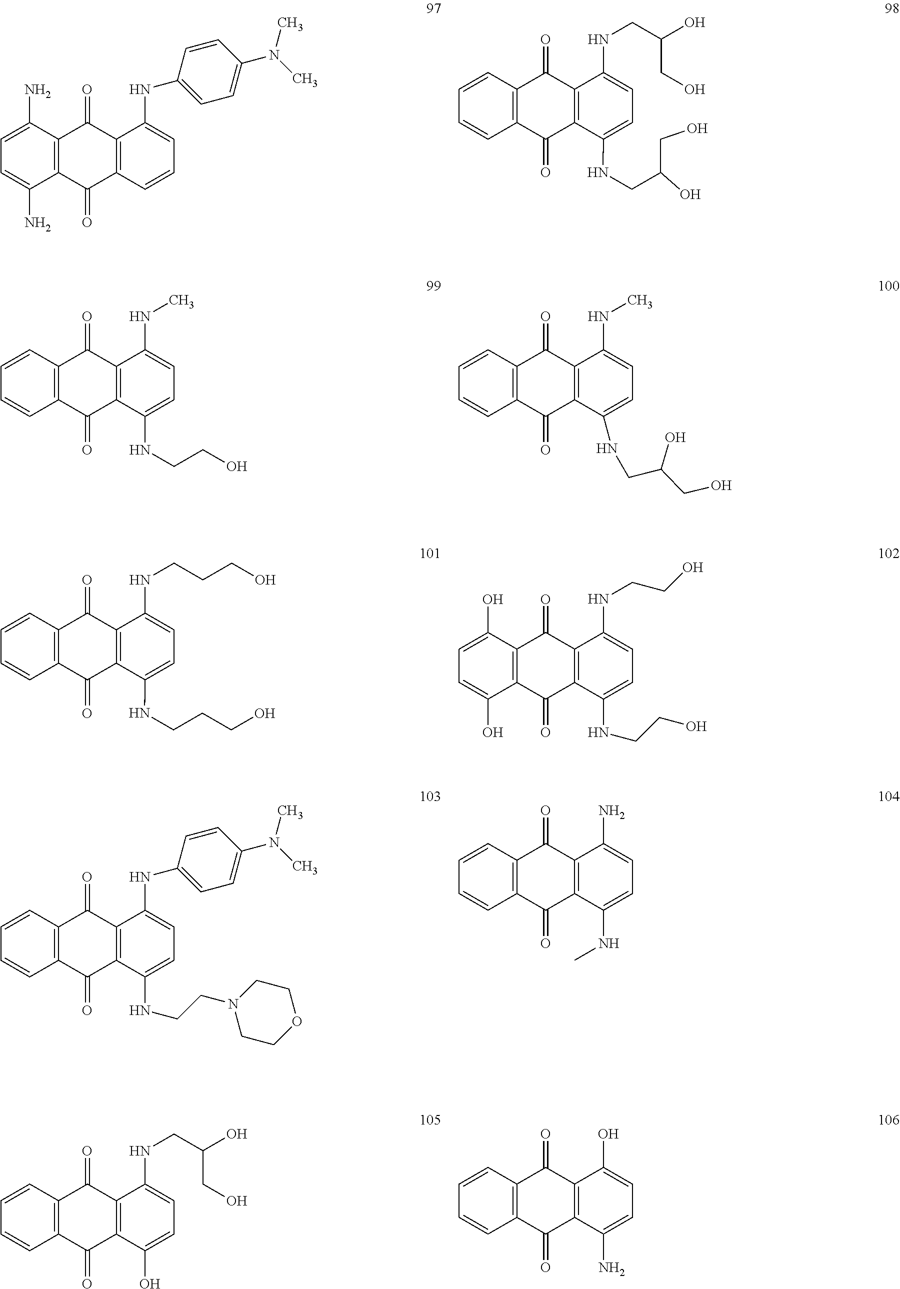 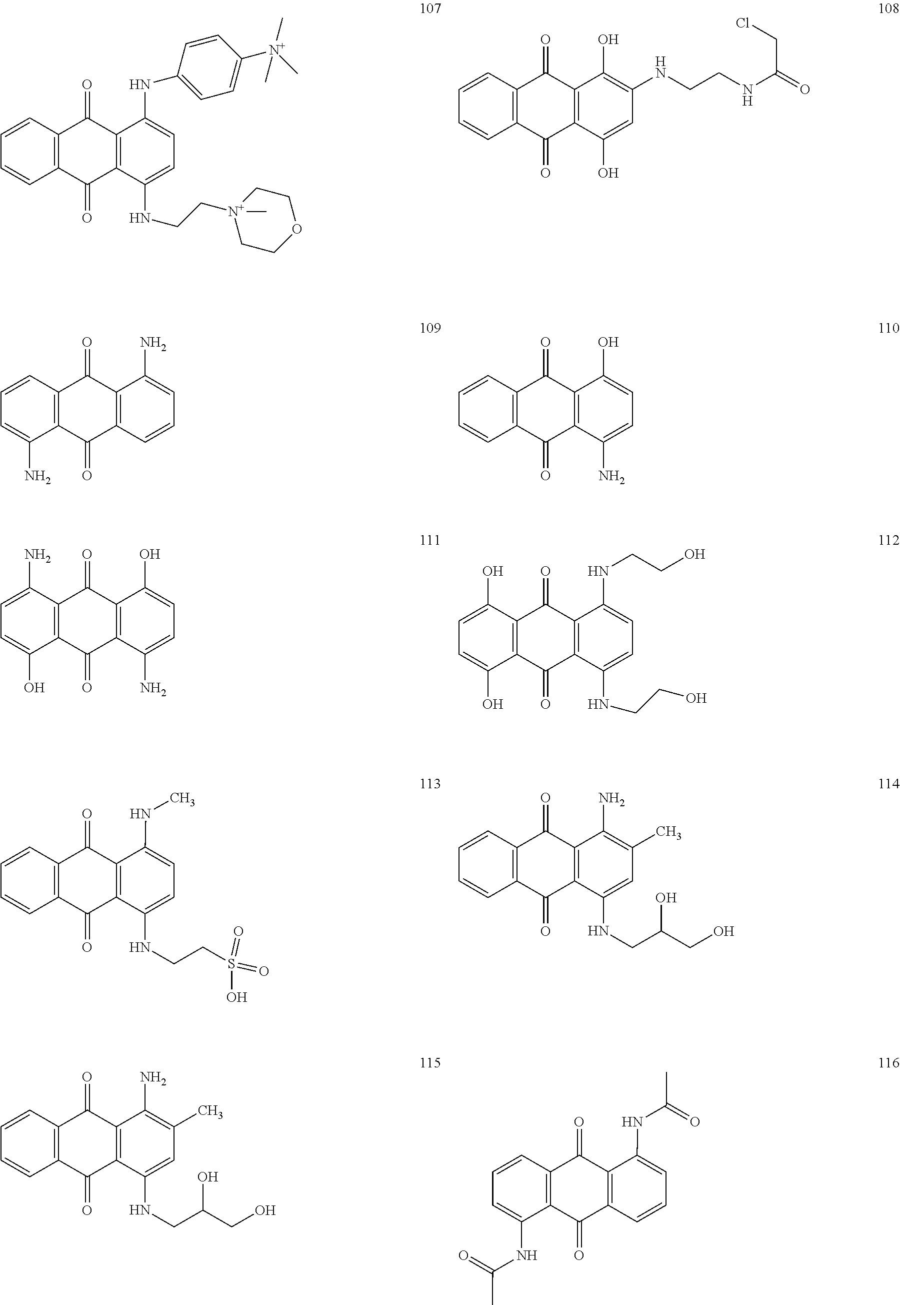 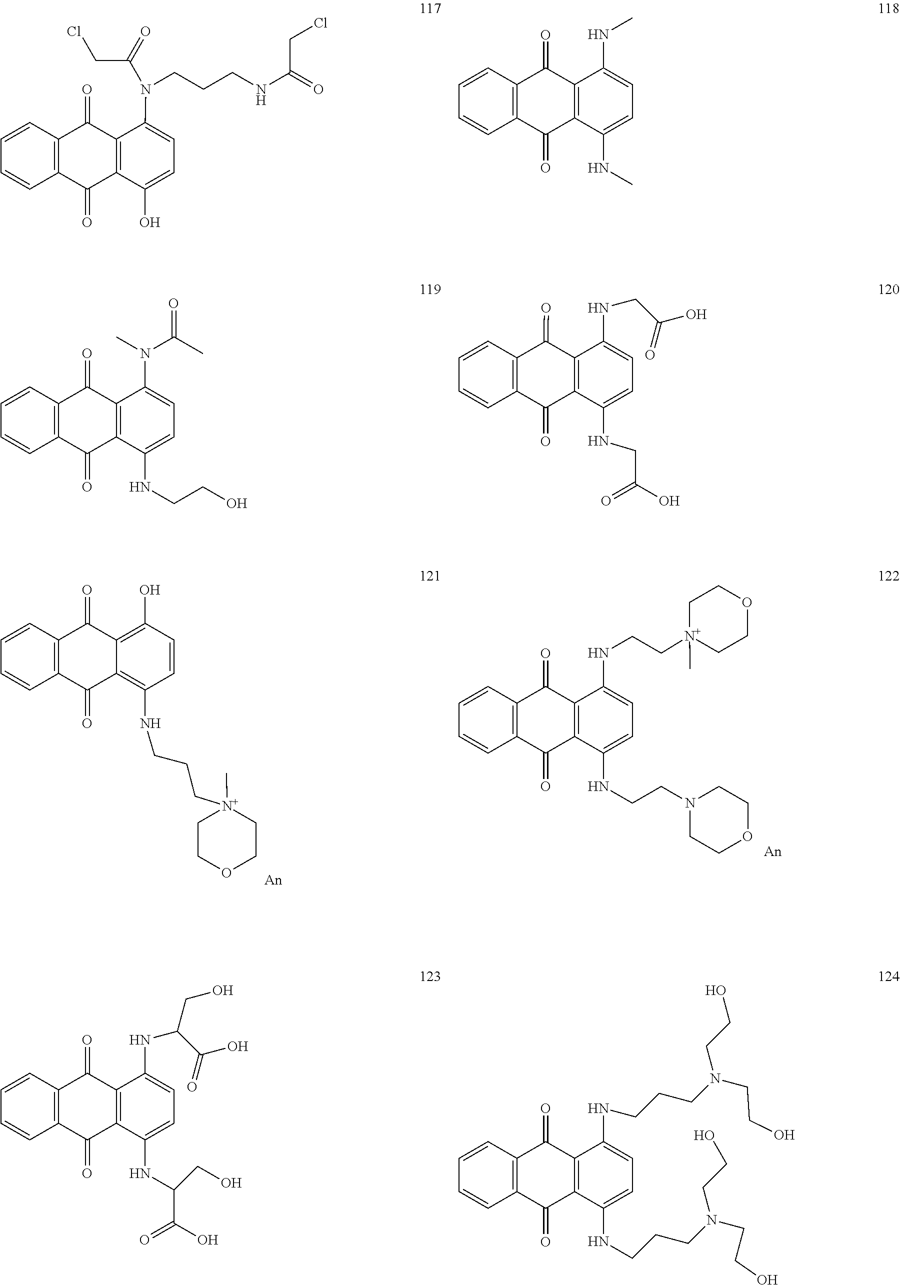 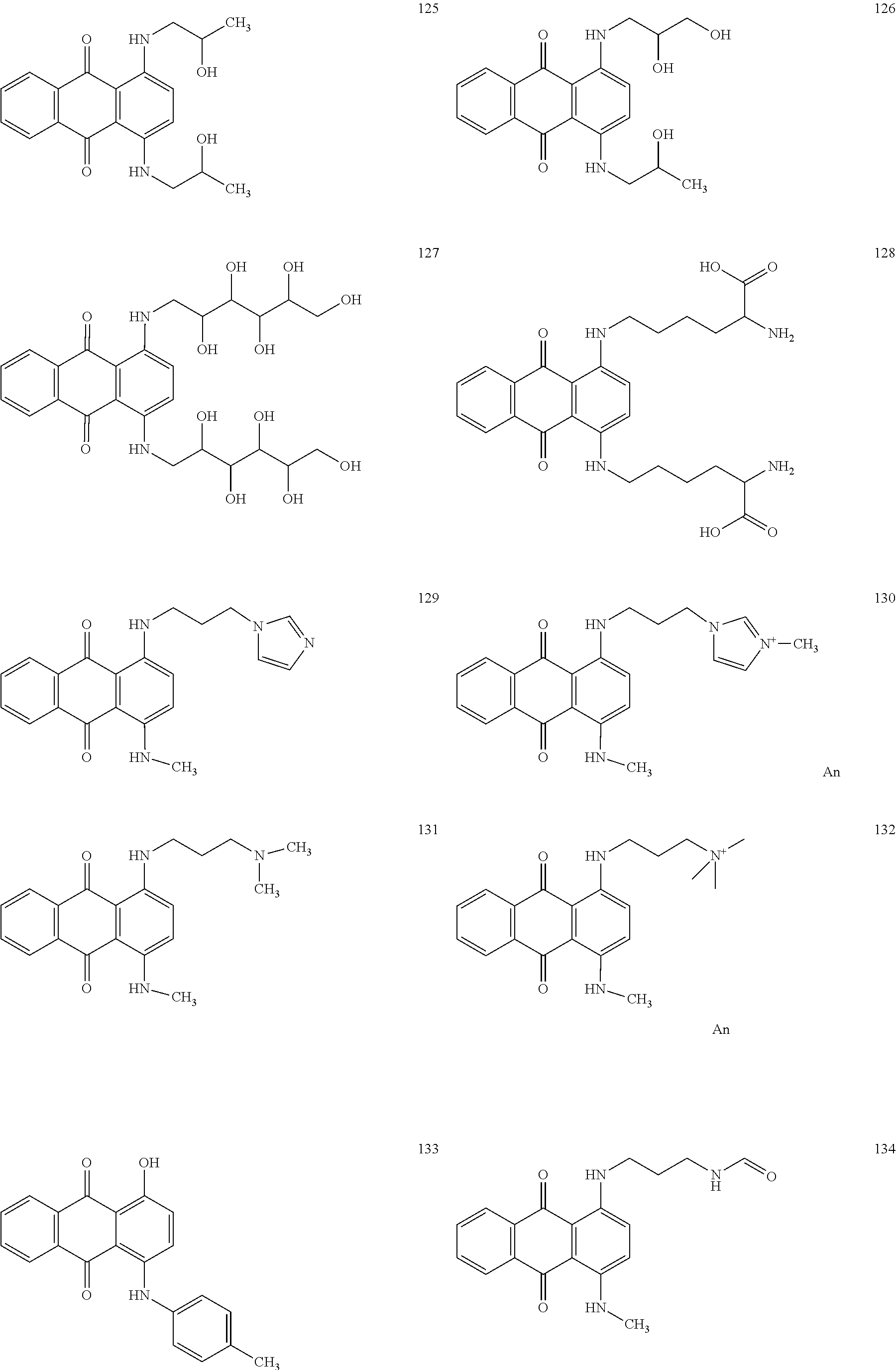 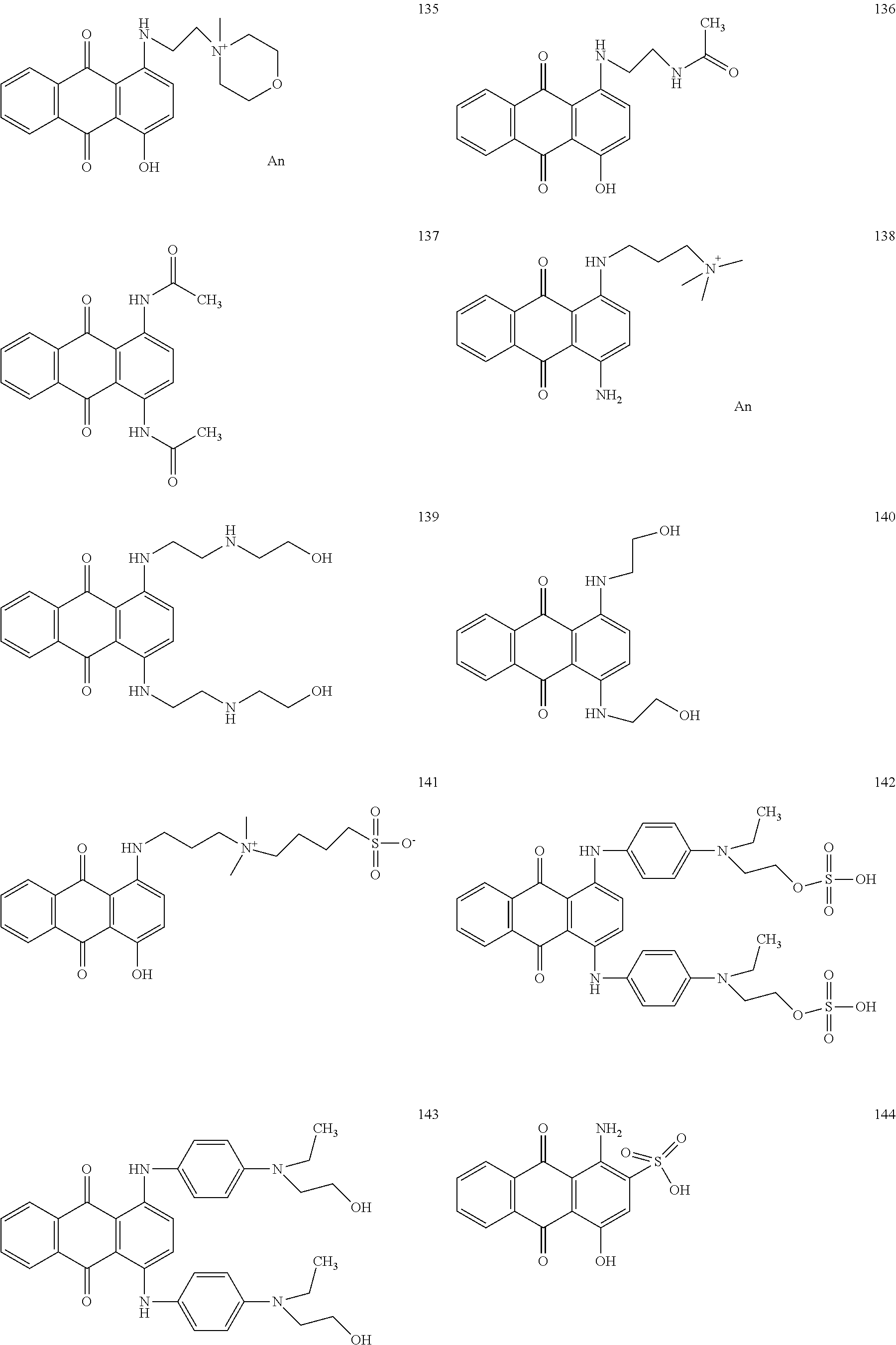 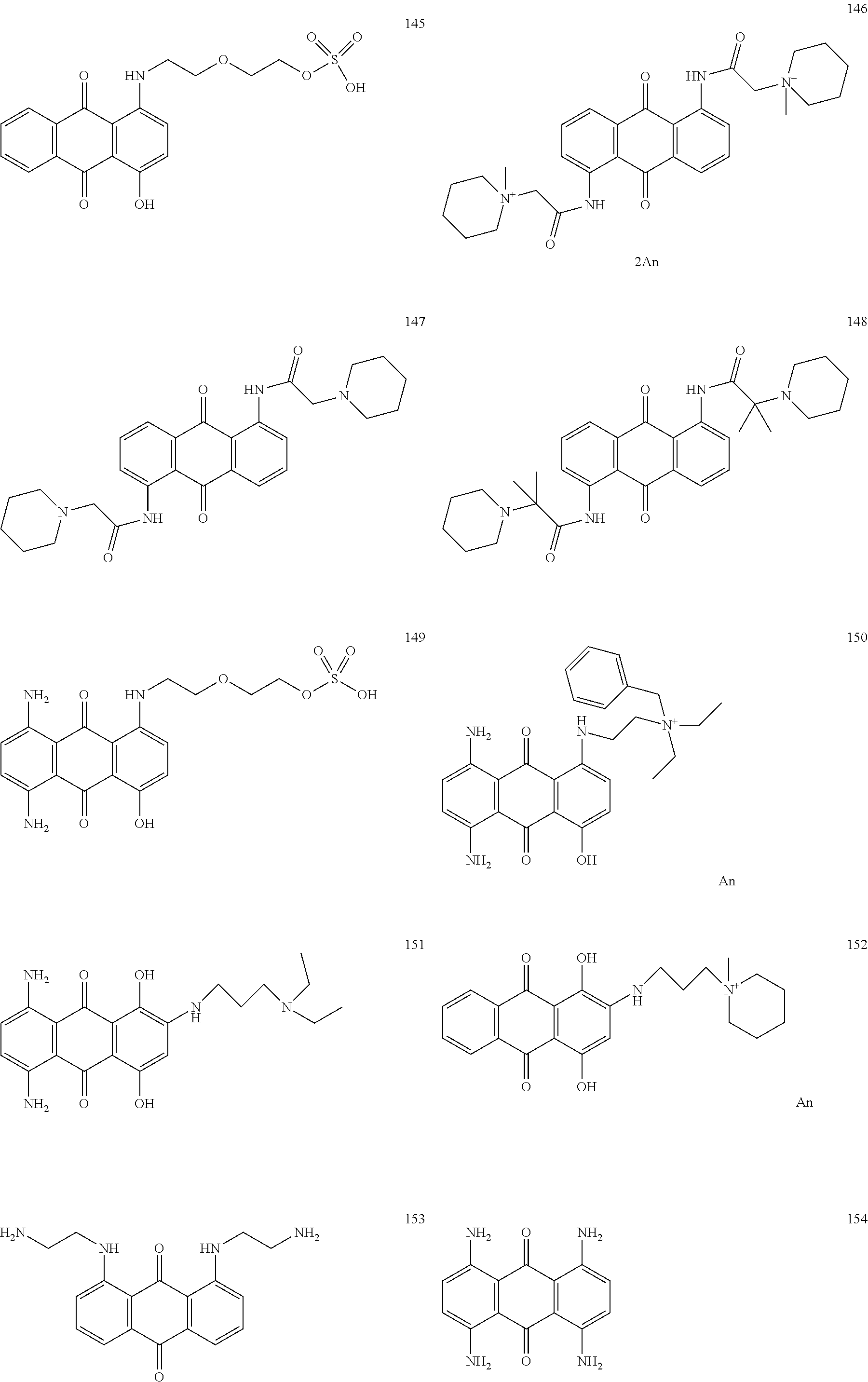 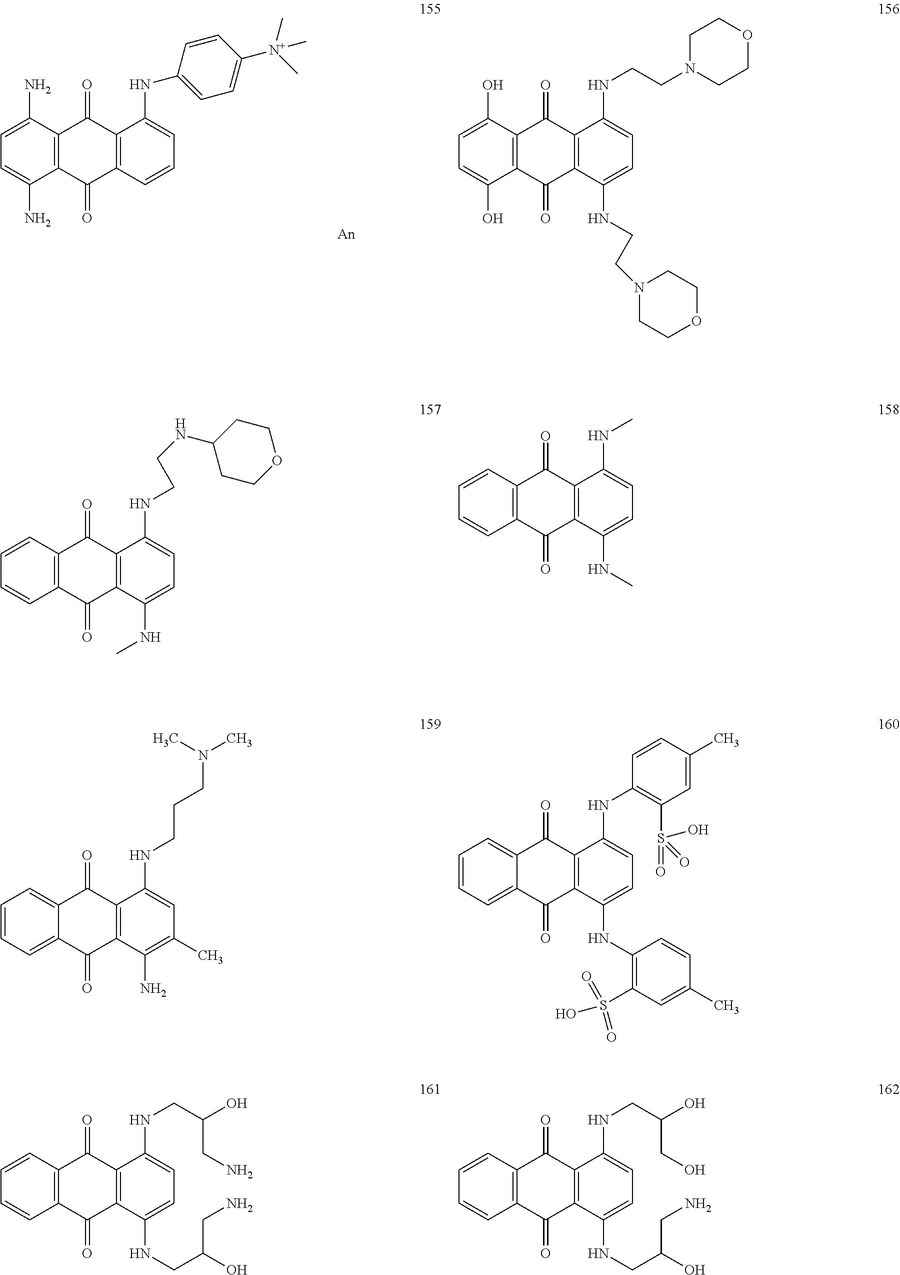 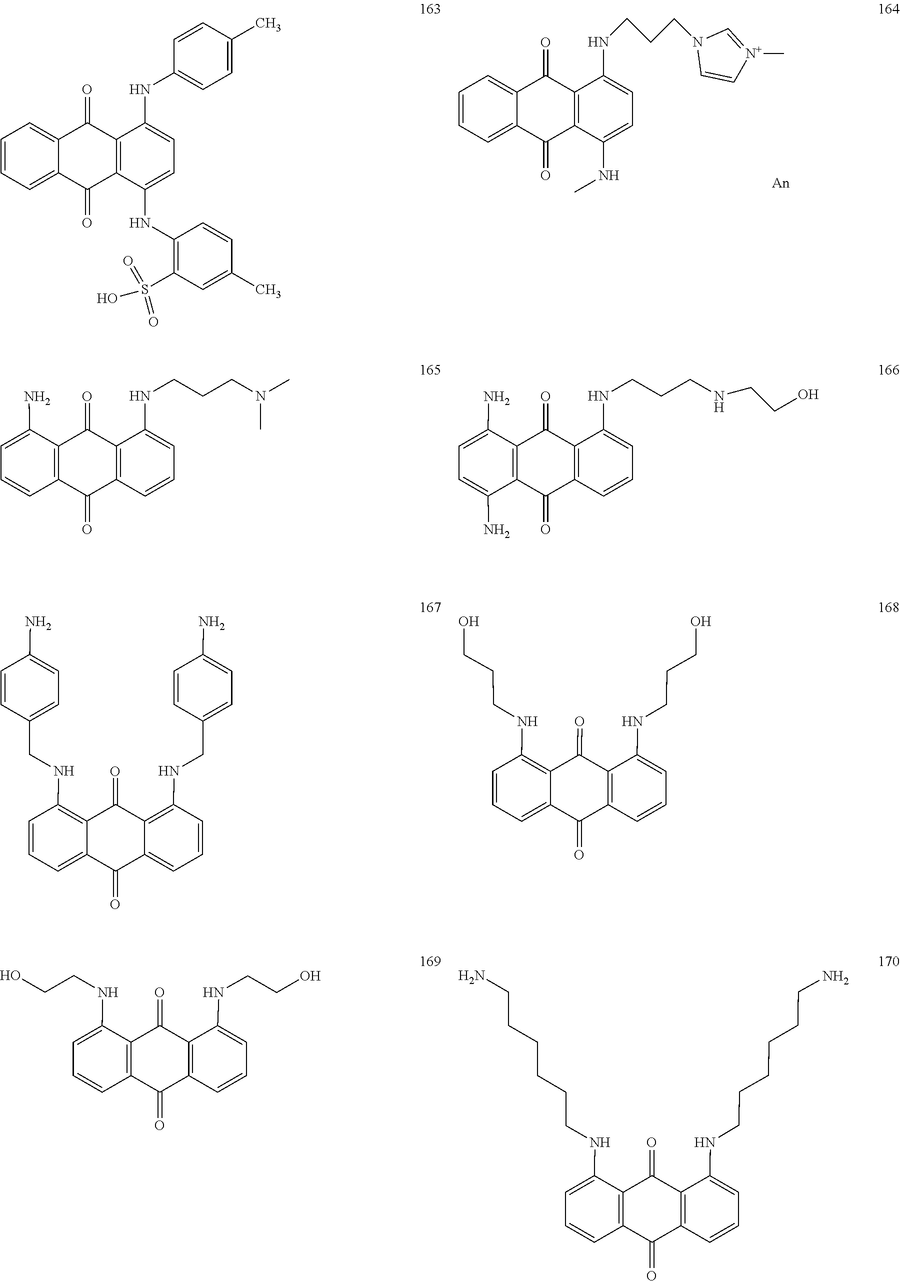 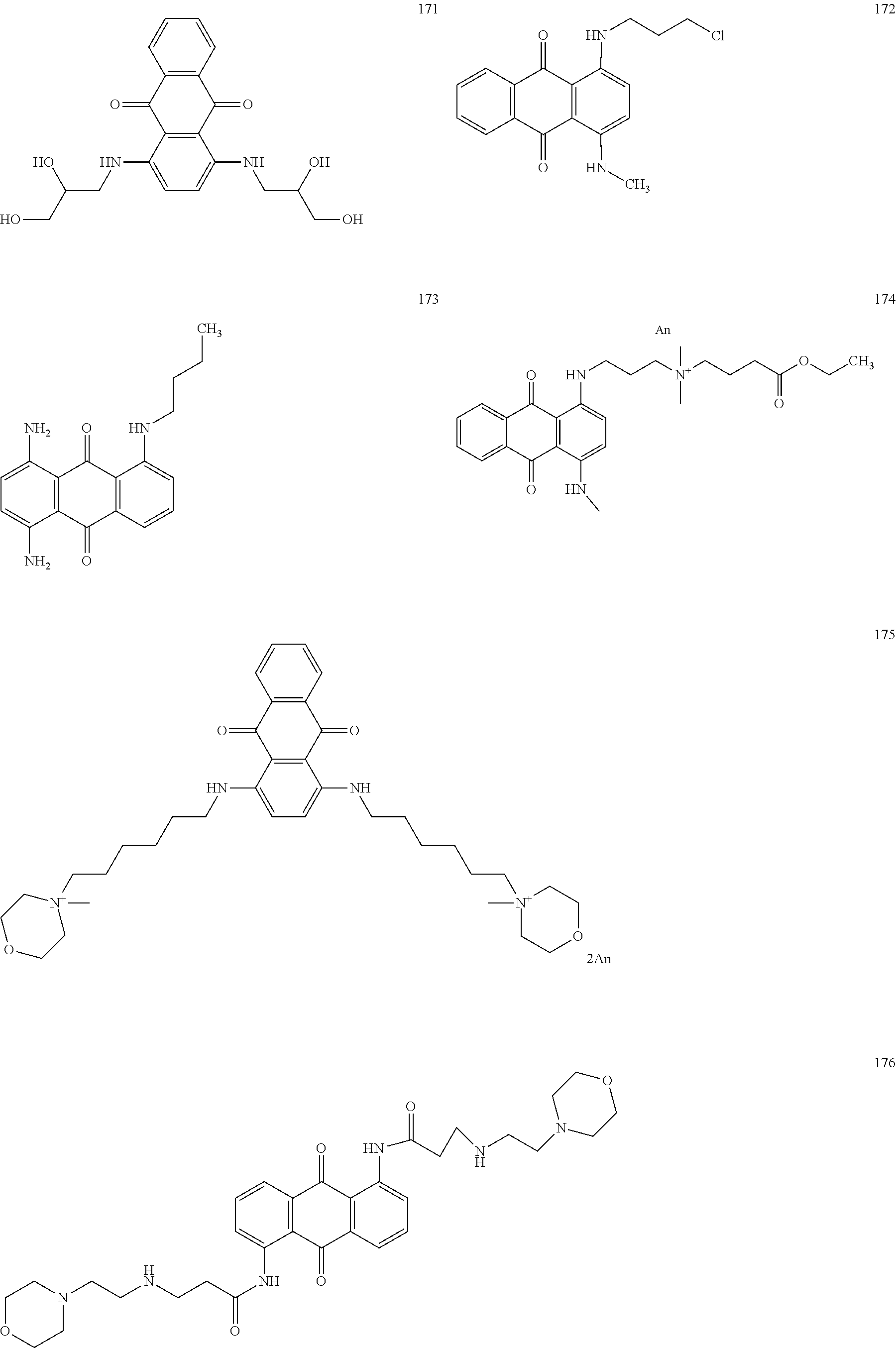 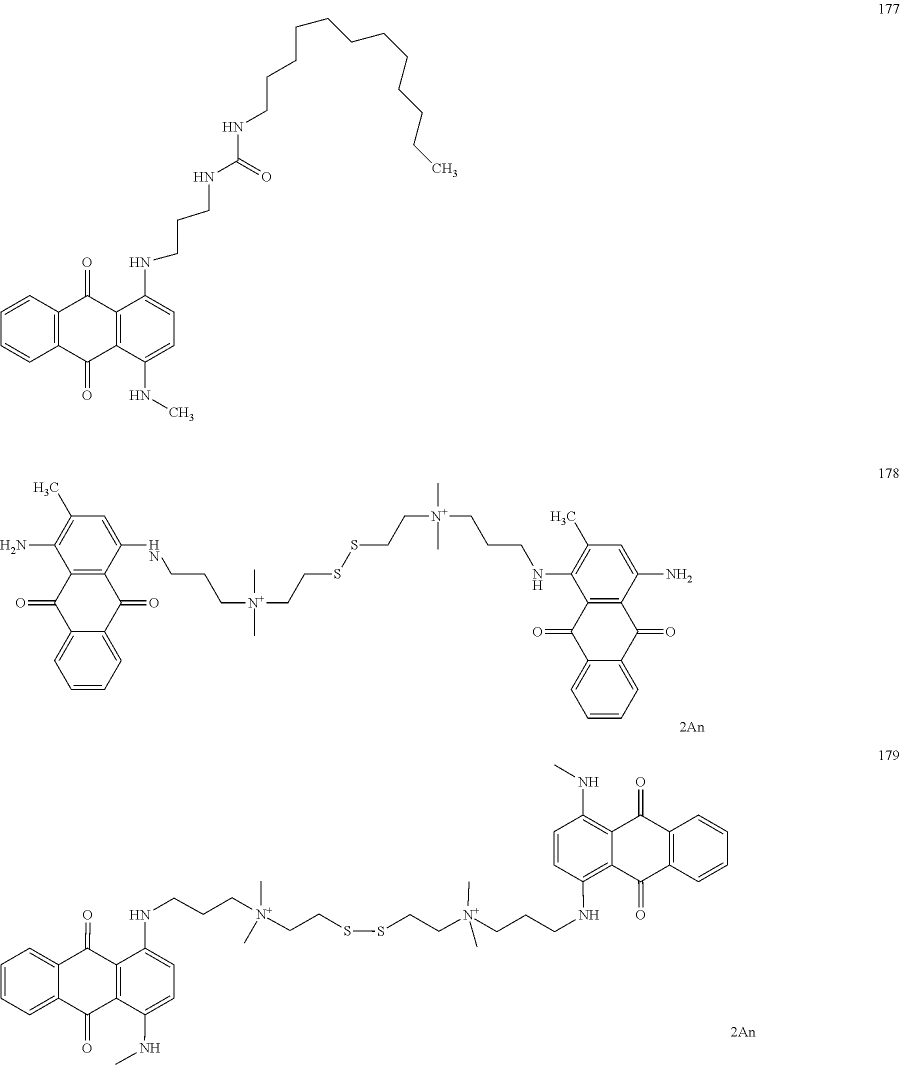 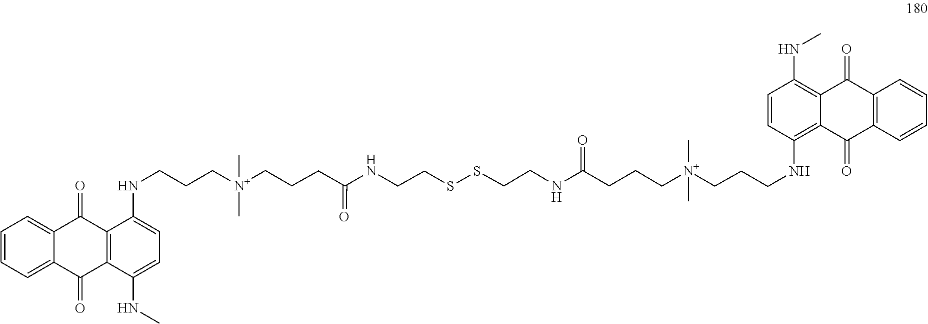
wherein An, which may be identical or different, represents an anionic counterion chosen from mesylate, tosylate, or halide.

40. The process according to claim 28, wherein the at least one cationic (poly)methine fluorescent dye is chosen from direct dyes chosen from cyanin dyes, styryl/hemicyanin dyes, naphthalimide dyes, or mixtures thereof.

41. The process according to claim 28, wherein the at least one cationic (poly)methine fluorescent dye is chosen from dyes which absorb light in the yellow, orange and red range.

42. The process according to claim 28, wherein the at least one cationic (poly)methine fluorescent dye is chosen from dyes which bear at least one cationic chromophore chosen from formulae (III), (IV), (IIIa), or (IVa) below:
W⁺—[C(R^{c})═C(R^{d})]_{m′}—Ar′-(*)Q⁻ (I)
Ar—[C(R^{d})═C(R^{c})]_{m′}—W′⁺-(*)Q⁻ (IV),
wherein in formulae (III) and (IV):
W⁺ represents a cationic heteroaryl group comprising a quaternary ammonium optionally substituted with one or more (C₁-C₈)alkyl groups optionally substituted with one or more hydroxyl groups;
W′⁺ represents a divalent heteroaryl radical as defined for W⁺;
Ar represents an aryl group, optionally substituted with i) one or more halogen atoms such as chlorine or fluorine; ii) one or more (C₁-C₈)alkyl; iii) one or more hydroxyl groups; iv) one or more (C₁-C₈)alkoxy groups; v) one or more hydroxy(C₁-C₈)alkyl groups, vi) one or more amino or (di)(C₁-C₈)alkylamino groups, vii) with one or more acylamino groups; viii) one or more heterocycloalkyl groups;
Ar′ is a divalent aryl radical;
m′ represents an integer between 1 and 4 inclusive;
R^{c} and R^{d}, which may be identical or different, represent a hydrogen atom or optionally a substituted (C₁-C₈)alkyl, or alternatively R^{c} is contiguous with W or W′ and/or R^{d} is contiguous with Ar or Ar′ and form, with the atoms that bear them, a (hetero)cycloalkyl;
Q⁻ is an organic or mineral anionic counterion;
(*) represents the part of the fluorescent chromophore that is bonded to the rest of the dye, 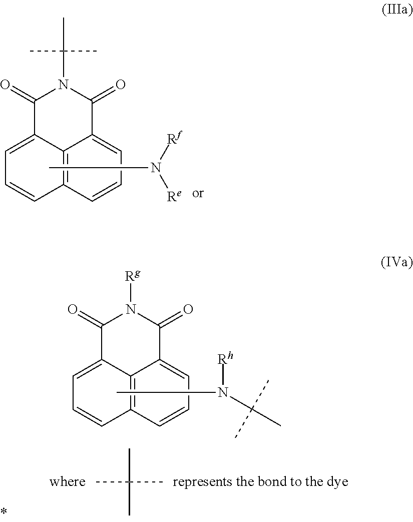
wherein in formulae (IIIa) and (IVa) R^{e}, R^{f}, R^{g} and R^{h}, which may be identical or different, represent a hydrogen atom or a (C₁-C₆)alkyl group which is optionally substituted.

43. The process according to claim 28, wherein the at least one cationic (poly)methine fluorescent dye is chosen from the dyes of formulae (V), (VI), or (VII): 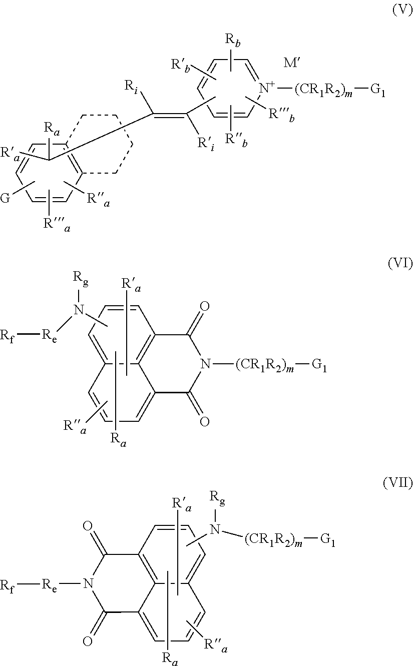
organic or mineral acid or base salts thereof, optical isomers, geometrical isomers thereof, tautomers thereof, or solvates thereof;
wherein:
R₁ and R₂, which may be identical or different, represent a hydrogen atom or a C₁-C₆ alkyl group;
G₁ represents a hydrogen atom or a group chosen from NH₂ and OH;
Rₐ, R′ₐ, R″ₐ, R′″ₐ, R_{b}, R′_{b}, R″_{b}, and R′″_{b}, which may be identical or different, represent a) a hydrogen atom, b) a halogen atom, a group chosen from: c) amino, d) (C₁-C₄)alkylamino, e) (C₁-C₄)dialkylamino, f) cyano, g) carboxyl —C(O)OH or carboxylate —C(O)O⁻, Q⁺, h) hydroxy —OH or alkoxide —O⁻Q⁺, i) (poly)halo(C₁-C₆)alkyl j) acylamino, k) (C₁-C₆)alkoxy, l) (C₁-C₆)alkylthio, m) (poly)hydroxy(C₂-C₄)alkoxy, n) (C₁-C₆)alkylcarbonyloxy, o) (C₁-C₆)alkoxycarbonyl, p) (C₁-C₆)alkylcarbonylamino, q) acylamino, r) carbamoyl, s) (C₁-C₆)alkylsulfonylamino, t) aminosulfonyl, u) —SO₃H or sulfonate —SO₃⁻, Q⁺ or v) (C₁-C₆)alkyl optionally substituted with a group chosen from (C₁-C₆)alkoxy, hydroxyl, cyano, carboxyl, amino, (di)(C₁-C₄)alkylamino, or alternatively the two alkyl radicals borne by the nitrogen atom of the amino group form a 5- to 7-membered heterocycle optionally comprising another nitrogen or non-nitrogen heteroatom;
or alternatively two groups Rₐ and R′ₐ; R_{b} and R′_{b}, borne by two adjacent carbon atoms, together form a benzo or indeno ring, a fused heterocycloalkyl or fused heteroaryl group; the benzo, indeno, heterocycloalkyl or heteroaryl ring being optionally substituted with a halogen atom, an amino, (C₁-C₄)alkylamino, (C₁-C₄)dialkylamino, nitro, cyano, carboxyl, hydroxyl or trifluoromethyl group, an acylamino, (C₁-C₄)alkoxy (poly)hydroxy(C₁-C₄)alkoxy, (C₁-C₄)alkylcarbonyloxy, (C₁-C₄)alkoxycarbonyl or (C₁-C₄)alkylcarbonylamino radical, an acylamino, carbamoyl or alkoxyalkylsulfonylamino radical, an aminosulfonyl radical, or a (C₁-C₆)alkyl radical optionally substituted with: a group chosen from (C₁-C₆)alkoxy, hydroxyl, cyano, carboxyl, amino, (C₁-C₄)alkylamino, or (C₁-C₄)dialkylamino, or alternatively the two alkyl radicals borne by the nitrogen atom of the amino group form a 5- to 7-membered heterocycle optionally comprising another nitrogen or non-nitrogen heteroatom;
or alternatively, two groups Rᵢ and Rₐ; and/or a group R′ᵢ and R′ₐ together form a fused (hetero)cycloalkyl;
R_{g} represents a hydrogen atom, a (hetero)aryl(C₁-C₄)alkyl group or a (C₁-C₆)alkyl group that is optionally substituted;
Rₑ represents a covalent bond, a linear or branched, optionally substituted (C₁-C₈)alkylene or (C₂-C₈)alkenylene hydrocarbon-based chain
R_{f} represents a hydrogen atom, a (C₁-C₄)alkoxy group, an amino group R₃R₄N—, a quaternary ammonium group M′, R₃R₄R₅N⁺— in which R₃, R₄ and R₅, which may be identical or different, represent a hydrogen atom or a (C₁-C₄)alkyl group or R₃R₄N— represents an optionally substituted heteroaryl group, or alternatively M′, R₃R₄R₅N⁺— represents an optionally substituted cationic heteroaryl group;
G represents a group i) —NR_{c}R_{d}, ii) —OR with R representing a) a hydrogen atom, b) an optionally substituted, preferentially unsubstituted (C₁-C₆)alkyl group, c) an optionally substituted (hetero)aryl group, d) an optionally substituted (hetero)aryl(C₁-C₆)alkyl group such as benzyl, e) optionally substituted (hetero)cycloalkyl group, f) optionally substituted (hetero)cycloalkyl(C₁-C₆)alkyl group;
or alternatively when G represents —NR_{c}R_{d}, two groups R_{c} and R′ₐ and/or R_{d} and Rₐ together form a saturated heteroaryl or heterocycle, optionally substituted with one or more (C₁-C₆)alkyl groups;
R_{c} and R_{d}, which may be identical or different, represent a hydrogen atom or a group chosen from: a) optionally substituted (hetero)aryl, b) optionally substituted (hetero)aryl(C₁-C₄)alkyl, c) optionally substituted (hetero)cycloalkyl, d) optionally substituted (hetero)cycloalkyl(C₁-C₄)alkyl, f) (C₂-C₅)alkyl, or g) (C₁-C₈)alkyl which is optionally substituted;
or alternatively two adjacent radicals R_{c} and R_{d} borne by the same nitrogen atom together form an optionally substituted heterocyclic or optionally substituted heteroaryl group;
Rᵢ and R′ᵢ, which may be identical or different, represent a hydrogen atom or a (C₁-C₄)alkyl group; 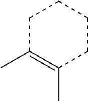
represents a (hetero)aryl group fused to the phenyl ring; or alternatively is absent from the phenyl;
m represents an integer between 1 and 18 inclusive;
M′ represents an anionic counterion, derived from a salt of an organic or mineral acid, or from an organic or mineral base that ensures the electrical neutrality of the molecule;
Q⁺ represents a cationic counterion, derived from a salt of an organic or mineral acid, or from an organic or mineral base that ensures the electrical neutrality of the molecule such as alkali metal, alkaline-earth metal or ammonium;
wherein when the molecule comprises a carboxylate, sulfonate or alkoxide group, then M′ and Q⁺ may be absent to ensure the electrical neutrality of said molecule.

44. The process according to claim 28, wherein the at least one cationic (poly)methine fluorescent dye is chosen from styryl dyes of formula (VIII) below: 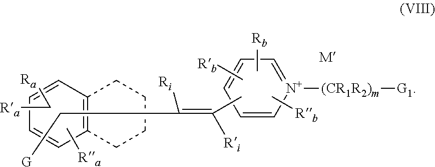
organic or mineral acid or base salts thereof, optical isomers thereof, geometrical isomers thereof, tautomers thereof, or solvates thereof;
wherein
R₁ and R₂, which may be identical or different, represent a hydrogen atom;
Rᵢ and R_{i′}, which may be identical or different, represent a hydrogen atom or a (C₁-C₄)alkyl group;
Rₐ, R′ₐ and R″ₐ, which may be identical or different, represent a hydrogen atom, a halogen atom, or an —OH, —O⁻Q⁺, (C₁-C₆)alkoxy, nitro, or cyano group, wherein Q⁺ represents a cationic counterion, derived from a salt of an organic or mineral acid, or from an organic or mineral base that ensures the electrical neutrality of the molecule;
R_{b}, R′_{b} and R″_{b}, which may be identical or different, represent a hydrogen atom or a (C₁-C₆)alkyl group;
or alternatively two contiguous radicals R_{b} and R′_{b} form, together with the carbon atoms that bear them, a benzo group that is condensed or fused to the pyridinium group, said benzo group optionally substituted;
G represents a group —NR_{c}R_{d} or (C₁-C₆)alkoxy group which is optionally substituted;
G₁ represents a hydrogen atom or an OH or NH₂ group;
Rᵢ and R′ᵢ, which may be identical or different, represent a hydrogen atom or a (C₁-C₄)alkyl group; 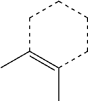
represents an aryl or heteroaryl group fused to the phenyl ring; or alternatively is absent from the phenyl ring;
m represents an integer between 1 and 18 inclusive;
R_{c} and R_{d}, which may be identical or different, represent a hydrogen atom, a (C₂-C₄)alkyl group or a substituted (C₁-C₈)alkyl group; and
M′ represents an anionic counterion, derived from a salt of an organic or mineral acid, or from an organic or mineral base that ensures the electrical neutrality of the molecule;
wherein when the molecule comprises an alkoxide group, then M′ and Q⁺ are optionally absent to ensure the electrical neutrality of said molecule.

45. The process according to claim 28, wherein the at least one cationic (poly)methine fluorescent dye is chosen from the dyes of the compounds of formulae (X), (XI), (XII), or (XIII) below: 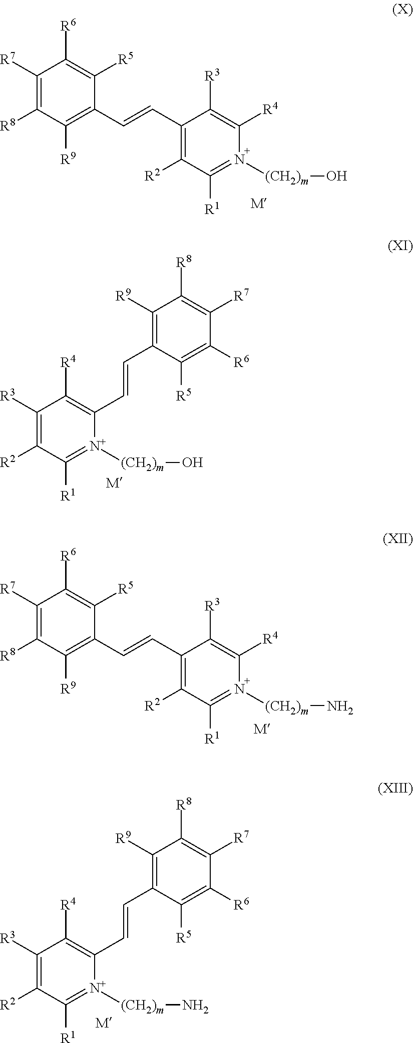
organic or mineral acid salts thereof, optical isomers thereof, geometrical isomers thereof, tautomers thereof, or solvates thereof;
wherein in formula (X), (XI), (XIII), or (XIII):
R¹, R², R³ and R⁴, which may be identical or different, represent a hydrogen atom or a (C₁-C₆)alkyl group;
R⁵, R⁶, R⁷, R⁸ and R⁹, which may be identical or different, represent i) a hydrogen atom or ii) a halogen atom, iii) a group OR in which R represents a hydrogen atom or Q⁺ represents a cationic counterion, derived from a salt of an organic or mineral acid, or from an organic or mineral base that ensures the electrical neutrality of the molecule, or a (C₁-C₃)alkyl group, iv) aryl, v) an aryl(C₁-C₃)alkyl group, vi) cyano, vii) nitro, viii) (C₁-C₃)alkylthio, ix) amino NR¹⁰R¹¹ with R¹⁰ and R¹¹, which may be identical or different, representing a) a hydrogen atom, b) a (C₂-C₄)alkyl group or c) a substituted (C₁-C₈)alkyl group:
cyano,
(C₁-C₃)alkoxy,
hydroxyl, and
(C₁-C₃)alkylcarbonyl;
m represents an integer between 1 and 18 inclusive;
M′ represents an anionic counterion, derived from a salt of an organic or mineral acid, or from an organic or mineral base that ensures the electrical neutrality of the molecule;
wherein when the molecule comprises an alkoxide group, then M′ and Q⁺ are optionally absent to ensure the electrical neutrality of the molecule.

46. The process according to claim 28, wherein the at least one cationic (poly)methine fluorescent dye is chosen from those of formulae (XIV) or (XV) below: 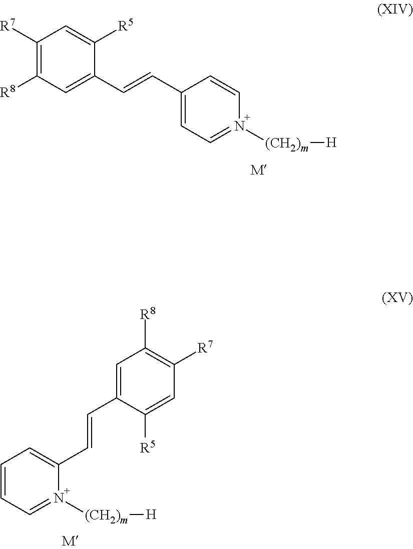
wherein:
R⁵ and R⁸, which may be identical or different, represent a hydrogen atom or a (C₁-C₄)alkoxy group;
R⁷ represents a (C₁-C₄)alkoxy group or NR¹⁰R¹¹ with R¹⁰ and R¹¹, which may be identical or different, representing a) a hydrogen atom, or b) a (C₁-C₈)alkyl group optionally substituted with one or more groups chosen from i) hydroxyl, ii) R—Z—C(X)—Y— with X, Y and Z representing an oxygen or sulfur atom or N(R′), or alternatively X and/or Z represent a bond, R and R′, which may be identical or different, represent a hydrogen atom or a (C₁-C₆)alkyl group, iii) sulfonic SO₃H, iv) sulfonate SO₃⁻, Q⁺, v) carboxylate C(O)O⁻, Q⁺ with Q⁺ representing a cationic counterion;
m represents an integer between 1 and 18 inclusive; and
M′ represents an anionic counterion, derived from a salt of an organic or mineral acid, or from an organic or mineral base that ensures the electrical neutrality of the molecule;
wherein when the molecule comprises an alkoxide group, then M′ and Q⁺ are optionally absent to ensure the electrical neutrality of said molecule.

47. The process according to claim 46, wherein:
the at least one cationic (poly)methine fluorescent dye is chosen from compounds of formula (X), wherein:

| R¹ | R² | R³ | R⁴ | R⁵ | R⁶ | R⁷ | R⁸ | R⁹ | m |
|---|---|---|---|---|---|---|---|---|---|
| H | H | H | H | H | H | H | H | H | 1 |
| H | H | H | H | H | t-Bu | OH | t-Bu | H | 5 |
| H | H | H | H | H | t-Bu | OH | t-Bu | H | 5 |
| H | H | H | H | H | H | NH₂ | H | H | 1 |
| H | H | H | H | H | H | NH₂ | H | OCH₃ | 1 |
| H | H | H | H | H | H | OH | Br | H | 5 |
| H | H | H | H | H | OCH₃ | OH | OCH₃ | H | 5 |
| H | H | H | H | Cl | H | OH | H | Cl | 5 |
| H | H | H | H | H | H | OH | H | H | 10 |
| H | H | H | H | H | OCH₃ | OH | OCH₃ | H | 10 |
| H | H | H | H | H | t-Bu | OH | t-Bu | H | 10 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | t-Bu | OH | t-Bu | H | 10 |
| H | H | H | H | H | H | N(CH₂CH₃)CH₂CH₂OH | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | N(CH₂CH₃)₂ | H | H | 1 |
| H | H | H | H | H | H | n-C₆H₁₃ | H | H | 1 |
| H | H | H | H | H | H | N(CH₂CH₂OH)₂ | H | H | 2 |
| H | H | H | H | H | H | N(n-Bu)₂ | H | H | 2 |
| H | H | H | H | H | OCH₃ | OH | H | H | 10 |
| H | H | H | H | H | H | OC₂H₅OH | H | H | 1 |
| H | H | H | H | H | H | OH | H | H | 1 |
| | | | | | | | | |
|---|---|---|---|---|---|---|---|---|
| H | H | benzo | H | H | H | H | H | 1 |
| H | H | benzo | H | H | N(CH₂CH₂OH)₂ | H | H | 1 |
| H | H | benzo | H | H | N(CH₂CH₂OH)₂ | H | H | 1 |

organic or mineral acid or base salts thereof, optical isomers, geometrical isomers thereof, tautomers thereof, or solvates thereof;
cationic (poly)methine fluorescent dyes of formula (XI), wherein:

| R¹ | R² | R³ | R⁴ | R⁵ | R⁶ | R⁷ | R⁸ | R⁹ | m |
|---|---|---|---|---|---|---|---|---|---|
| CH₃ | H | H | H | OH | H | OCH₃ | H | H | 2 |
| CH₃ | H | H | H | H | H | OCH₃ | OCH₂—Ph | H | 2 |
| CH₃ | H | H | H | H | H | H | H | OCH₃ | 2 |
| CH₃ | H | H | H | F | H | H | H | H | 2 |
| CH₃ | H | H | H | H | H | H | OPh | H | 2 |
| CH₃ | H | H | H | H | H | N(CH₂CH₂OAc)₂ | H | H | 2 |
| CH₃ | H | H | H | OCH₃ | H | OCH₃ | OCH₃ | H | 2 |
| CH₃ | H | H | H | H | H | H | CH₃ | H | 2 |
| CH₃ | H | H | H | H | H | OH | H | H | 2 |
| CH₃ | H | H | H | H | OCH₃ | OCH₃ | OCH₃ | H | 2 |
| CH₃ | H | H | H | H | OCH₃ | OH | OH | H | 2 |
| CH₃ | H | H | H | H | H | OCH₃ | OCH₃ | OCH₃ | 2 |
| CH₃ | H | H | H | H | H | H | OCH₃ | OH | 2 |
| CH₃ | H | H | H | H | H | N(n-butyl)₂ | H | H | 2 |
| CH₃ | H | H | H | H | OCH₃ | OCH₃ | H | H | 2 |
| CH₃ | H | H | H | H | H | H | H | H | 2 |
| CH₃ | H | H | H | H | H | i-propyl | H | H | 2 |
| H | H | H | H | H | H | OH | H | H | 6 |
| H | H | H | H | H | OCH₃ | OCH₃ | OCH₃ | H | 6 |
| H | H | H | H | OH | H | OCH₃ | H | H | 2 |
| H | H | H | H | OCH₃ | H | H | OCH₃ | OCH₃ | 2 |
| H | H | H | H | H | H | H | H | Br | 2 |
| H | H | H | H | H | H | OH | H | H | 2 |
| H | H | H | H | OCH₃ | OCH₃ | OCH₃ | H | H | 6 |
| H | H | H | H | OH | OCH₃ | H | H | H | 6 |
| H | H | H | H | H | OCH₃ | OCH₃ | H | H | 6 |
| H | H | H | H | H | OCH₃ | OCH₃ | H | H | 2 |
| H | H | H | H | H | H | C(O)—OH | H | H | 6 |
| H | H | H | H | H | H | C(O)—OH | H | H | 2 |
| H | H | H | H | H | H | i-propyl | H | H | 2 |
| H | H | H | H | H | H | N(CH₃)CH₂CH₂CN | H | H | 2 |
| H | H | H | H | H | H | OCH₃ | OCH₂Ph | H | 2 |
| H | H | H | H | H | H | H | OPh | H | 2 |
| H | H | H | H | H | H | N(CH₂CH₂C(O)CH₃)₂ | H | H | 2 |
| H | H | H | H | OH | H | OCH₃ | H | H | 6 |
| H | H | H | H | H | OCH₃ | OCH₃ | OCH₃ | H | 2 |
| H | H | H | H | OCH₃ | H | OCH₃ | OCH₃ | H | 6 |
| H | H | H | H | H | H | H | CH₃ | H | 2 |
| H | H | H | H | H | H | N(CH₃)CH₂CH₂OH | H | H | 2 |

organic or mineral acid or base salts thereof, geometrical isomers thereof, tautomers thereof, or solvates thereof;
cationic (poly)methine fluorescent dyes of formulae (X′) or (XI′): 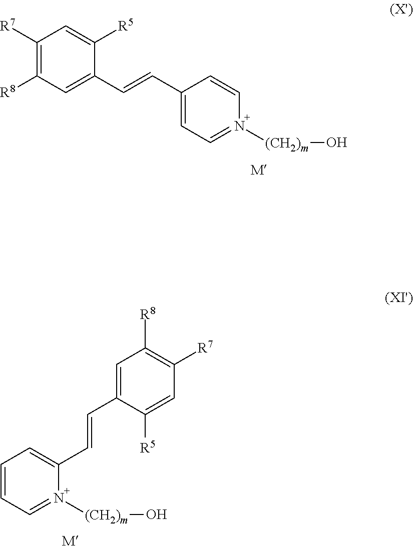
wherein

| R⁵ | R⁷ | R⁸ | m | | R⁵ | R⁷ | R⁸ | M |
|---|---|---|---|---|---|---|---|---|
| H | N(CH₂CH₂OH)₂ | H | 2 | and | OCH₃ | OCH₃ | OCH₃ | 2 |
| H | N(CH₂CH₂OH)₂ | H | 3 | | OCH₃ | OCH₃ | OCH₃ | 3 |
| H | N(CH₂CH₂OH)₂ | H | 4 | | OCH₃ | OCH₃ | OCH₃ | 3 |
| H | N(CH₂CH₂OH)₂ | H | 5 | | OCH₃ | OCH₃ | OCH₃ | 4 |
| H | N(CH₂CH₂OH)₂ | H | 6 | | OCH₃ | OCH₃ | OCH₃ | 5 |
| H | N(CH₂CH₂OH)₂ | H | 8 | | OCH₃ | OCH₃ | OCH₃ | 8 |
| H | N(CH₂CH₂OH)₂ | H | 10 | | OCH₃ | OCH₃ | OCH₃ | 10 |
| H | N(CH₂CH₂OH)₂ | H | 12 | | OCH₃ | OCH₃ | OCH₃ | 12 |
| H | N(CH₂CH₂OH)₂ | H | 14 | | OCH₃ | OCH₃ | OCH₃ | 14 |
| H | N(CH₂CH₂OH)₂ | H | 16 | | OCH₃ | OCH₃ | OCH₃ | 16 |
| H | CH₃CH₂N(CH₂CH₂OH) | H | 2 | | | | | |
| H | CH₃CH₂N(CH₂CH₂OH) | H | 4 | | | | | |

organic or mineral acid or base salts thereof, geometrical isomers thereof, tautomers thereof, or solvates thereof;
cationic (poly)methine fluorescent dyes of formula (XII) wherein:

| R¹ | R² | R³ | R⁴ | R⁵ | R⁶ | R⁷ | R⁸ | R⁹ | m |
|---|---|---|---|---|---|---|---|---|---|
| H | H | H | H | H | H | OCH₃ | OCH₃ | OCH₃ | 2 |
| H | H | H | H | OH | OCH₃ | H | H | H | 2 |
| H | H | H | H | H | H | H | H | H | 2 |
| H | H | H | H | H | OCH₃ | OCH₃ | H | H | 2 |
| H | H | H | H | OH | H | OH | H | H | 6 |
| H | H | H | H | OCH₃ | H | OCH₃ | OCH₃ | H | 6 |
| H | H | H | H | H | H | OH | H | H | 6 |
| H | H | H | H | OCH₃ | H | H | H | F | 2 |
| H | H | H | H | H | H | C(O)—OH | H | H | 2 |
| H | H | H | H | H | H | Isopropyl | H | H | 2 |
| H | H | H | H | H | H | N(CH₂CH₂C(O)CH₃)₂ | H | H | 2 |
| H | H | H | H | OH | H | OCH₃ | H | H | 2 |
| H | H | H | H | H | H | OH | H | H | 2 |
| H | H | H | H | H | OCH₃ | OH | OH | H | 2 |
| H | H | H | H | H | CH₃ | OCH₂Ph | CH₃ | H | 2 |
| H | H | H | H | H | OCH₃ | OCH₃ | OCH₃ | H | 2 |
| H | H | H | H | H | OCH₃ | OCH₃ | OCH₃ | H | 6 |
| H | H | H | H | H | H | N(CH₃)₂ | H | H | 6 |
| H | H | H | H | H | H | OCH₃ | OCH₃ | OCH₃ | 6 |
| H | H | H | H | H | H | Phenyl | H | H | 6 |
| H | H | H | H | H | OCH₃ | OCH₃ | H | H | 6 |
| H | H | H | H | H | H | C(O)—OH | H | H | 6 |
| H | H | H | H | H | H | N(n-Butyl)₂ | H | H | 2 |
| H | H | H | H | H | H | OCH₃ | OCH₃ | H | 3 |
| H | H | H | H | H | H | OCH₃ | OCH₃ | H | 2 |
| H | H | H | H | H | H | OCH₃ | OCH₃ | H | 5 |
| H | H | H | H | H | H | OCH₃ | H | H | 3 |
| H | H | H | H | H | H | N(CH₃)₂ | H | H | 3 |
| H | H | H | H | H | H | H | H | H | 3 |

organic or mineral acid or base salts thereof, optical isomers thereof, geometrical isomers thereof, tautomers thereof, or solvates thereof;
cationic (poly)methine fluorescent dyes of formula (XIII), wherein:

| R¹ | R² | R³ | R⁴ | R⁵ | R⁶ | R⁷ | R⁸ | R⁹ | m |
|---|---|---|---|---|---|---|---|---|---|
| CH₃ | H | H | H | OH | H | OCH₃ | H | H | 2 |
| CH₃ | H | H | H | H | H | OCH₃ | OCH₂—Ph | H | 2 |
| CH₃ | H | H | H | H | H | H | H | OCH₃ | 2 |
| CH₃ | H | H | H | F | H | H | H | H | 2 |
| CH₃ | H | H | H | H | H | H | OPh | H | 2 |
| CH₃ | H | H | H | H | H | N(CH₂CH₂OAc)₂ | H | H | 2 |
| CH₃ | H | H | H | OCH₃ | H | OCH₃ | OCH₃ | H | 2 |
| CH₃ | H | H | H | H | H | H | CH₃ | H | 2 |
| CH₃ | H | H | H | H | H | OH | H | H | 2 |
| CH₃ | H | H | H | H | OCH₃ | OCH₃ | OCH₃ | H | 2 |
| CH₃ | H | H | H | H | OCH₃ | OH | OH | H | 2 |
| CH₃ | H | H | H | H | H | OCH₃ | OCH₃ | OCH₃ | 2 |
| CH₃ | H | H | H | H | H | H | OCH₃ | OH | 2 |
| CH₃ | H | H | H | H | H | N(n-butyl)₂ | H | H | 2 |
| CH₃ | H | H | H | H | OCH₃ | OCH₃ | H | H | 2 |
| CH₃ | H | H | H | H | H | H | H | H | 2 |
| CH₃ | H | H | H | H | H | i-propyl | H | H | 2 |
| H | H | H | H | H | H | OH | H | H | 6 |
| H | H | H | H | H | OCH₃ | OCH₃ | OCH₃ | H | 6 |
| H | H | H | H | OH | H | OCH₃ | H | H | 2 |
| H | H | H | H | OCH₃ | H | H | OCH₃ | OCH₃ | 2 |
| H | H | H | H | H | H | H | H | Br | 2 |
| H | H | H | H | H | H | OH | H | H | 2 |
| H | H | H | H | H | H | N(CH₃)₂ | H | H | 6 |
| H | H | H | H | OCH₃ | OCH₃ | OCH₃ | H | H | 6 |
| H | H | H | H | OH | OCH₃ | H | H | H | 6 |
| H | H | H | H | H | OCH₃ | OCH₃ | H | H | 6 |
| H | H | H | H | H | OCH₃ | OCH₃ | H | H | 2 |
| H | H | H | H | H | H | C(O)—OH | H | H | 6 |
| H | H | H | H | H | H | C(O)—OH | H | H | 2 |
| H | H | H | H | H | H | i-propyl | H | H | 2 |
| H | H | H | H | H | H | N(CH₃)CH₂CH₂CN | H | H | 2 |
| H | H | H | H | H | H | OCH₃ | OCH₂Ph | H | 2 |
| H | H | H | H | H | H | H | OPh | H | 2 |
| H | H | H | H | H | H | N(CH₂CH₂C(O)CH₃)₂ | H | H | 2 |
| H | H | H | H | OH | H | OCH₃ | H | H | 6 |
| H | H | H | H | H | OCH₃ | OCH₃ | OCH₃ | H | 2 |
| H | H | H | H | OCH₃ | H | OCH₃ | OCH₃ | H | 6 |
| H | H | H | H | H | H | H | CH₃ | H | 2 |
| H | H | H | H | H | H | N(CH₃)CH₂CH₂OH | H | H | 2 |
| CH₃ | H | H | H | H | H | N(CH₃)₂ | H | H | 2 |

organic or mineral acid or base salts thereof, geometrical isomers thereof, tautomers thereof, or the solvates thereof;
cationic (poly)methine fluorescent dyes of formulae (XII′) or (XIII′) below: 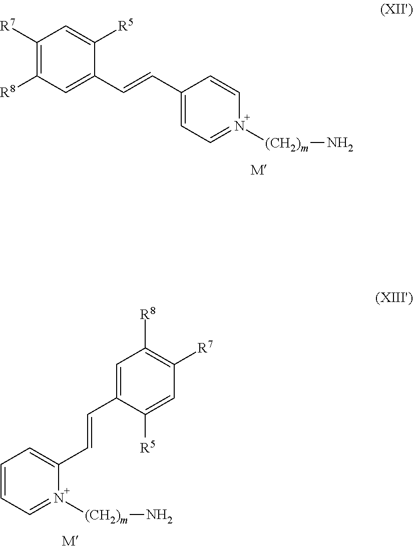
wherein:

| R⁵ | R⁷ | R⁸ | m | | R⁵ | R⁷ | R⁸ | m |
|---|---|---|---|---|---|---|---|---|
| H | N(CH₂CH₂OH)₂ | H | 2 | and | OCH₃ | OCH₃ | OCH₃ | 2 |
| H | N(CH₂CH₂OH)₂ | H | 3 | | OCH₃ | OCH₃ | OCH₃ | 3 |
| H | N(CH₂CH₂OH)₂ | H | 4 | | OCH₃ | OCH₃ | OCH₃ | 3 |
| H | N(CH₂CH₂OH)₂ | H | 5 | | OCH₃ | OCH₃ | OCH₃ | 4 |
| H | N(CH₂CH₂OH)₂ | H | 6 | | OCH₃ | OCH₃ | OCH₃ | 5 |
| H | N(CH₂CH₂OH)₂ | H | 8 | | OCH₃ | OCH₃ | OCH₃ | 8 |
| H | N(CH₂CH₂OH)₂ | H | 10 | | OCH₃ | OCH₃ | OCH₃ | 10 |
| H | N(CH₂CH₂OH)₂ | H | 12 | | OCH₃ | OCH₃ | OCH₃ | 12 |
| H | N(CH₂CH₂OH)₂ | H | 14 | | OCH₃ | OCH₃ | OCH₃ | 14 |
| H | N(CH₂CH₂OH)₂ | H | 16 | | OCH₃ | OCH₃ | OCH₃ | 16 |

organic or mineral acid or base salts thereof, geometrical isomers thereof, tautomers thereof, or solvates thereof;
cationic (poly)methine fluorescent dyes chosen from the following compounds: 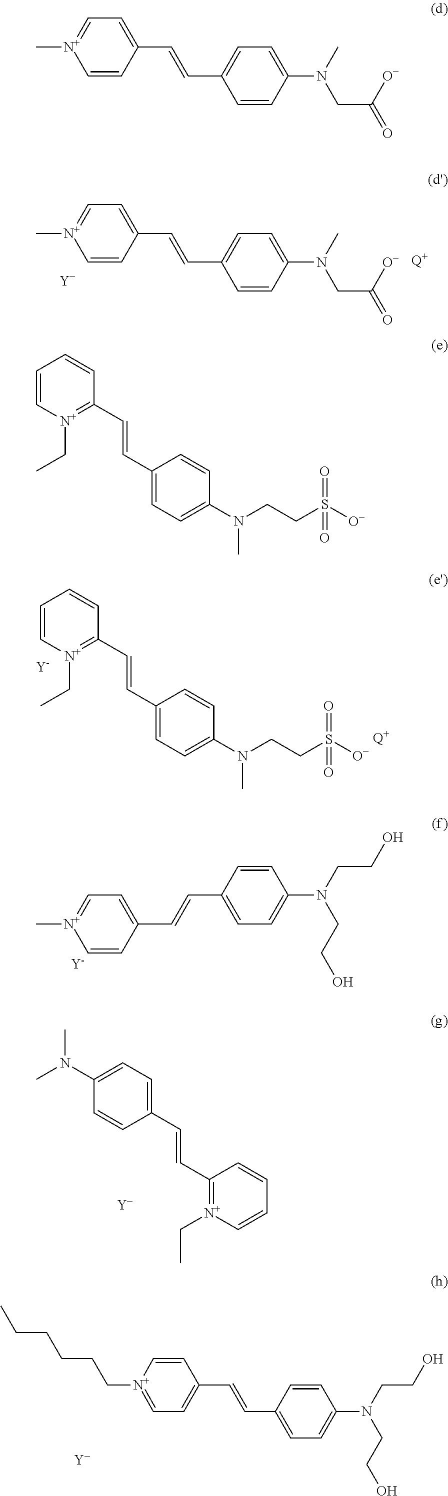
organic or mineral acid or base salts thereof, geometrical isomers thereof, tautomers thereof, or the solvates thereof.

48. The process according to claim 28, wherein the process further comprises applying to said keratin fibers a cosmetic composition comprising c) one or more reducing agents.

49. The process according to claim 28, wherein the process does not use any reducing agent.

50. The process according to claim 28, wherein the at least one anthraquinone compound and the at least one cationic (poly)methine fluorescent direct dye are applied together to the keratin fibers.

51. The process according to claim 28, wherein the process comprises at least two successive steps:
a step of applying to the keratin fibers a cosmetic composition comprising (b) at least one cationic (poly)methine fluorescent dye, followed by,
a step of applying to the keratin fibers a cosmetic composition comprising (a) at least one or more anthraquinone dye of formula (I) and/or (II).

52. The process according to claim 28, wherein the process comprises at least two successive steps:
a step of applying to the fibers a cosmetic composition comprising (a) at least one anthraquinone dye of formula (I) and/or (II), followed by
a step of applying to the keratin fibers a cosmetic composition comprising (b) at least one cationic (poly)methine fluorescent dye.

53. The process according to claim 28, wherein the pH of the cosmetic composition is between 6 and 11 inclusive.

54. A cosmetic composition comprising (a) at least one anthraquinone dye chosen from the compounds of formulae (I) and/or (II) below, optical isomers thereof, geometrical isomers thereof, tautomers thereof, solvates thereof, or mixtures thereof: 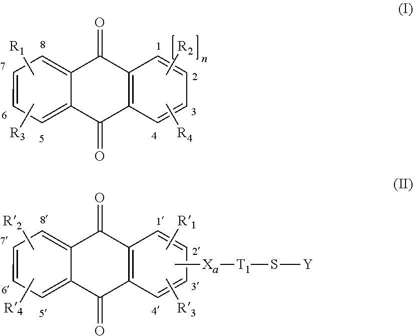
wherein in formula (I) and/or (II):
Xₐ, which may be identical or different, represents a bond, a heteroatom or a group chosen from an oxygen or sulfur atom, N(Rₐ), CO, SO, SO₂, or combinations thereof, with Rₐ representing a hydrogen atom or a (C₁-C₆)alkyl group optionally substituted with one or more hydroxyl groups;
Y represents: i) a hydrogen atom; ii) an alkali metal; iii) an alkaline-earth metal; iv) an ammonium group: N⁺R^{a}R^{b}R^{g}R^{d} or a phosphonium group: P⁺R^{a}R^{b}R^{g}R^{d} with R^{a}R^{b}, R^{g} and R^{d}, which may be identical or different, representing a hydrogen atom or a (C₁-C₄)alkyl group; or v) a thiol-function protecting group; or vi) the group (III) below: 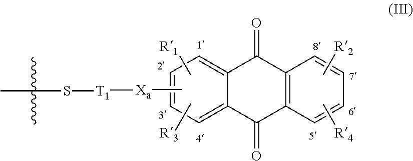
R₁, R₂, R₃, R₄, R′₁, R′₂, R′₃ and R′₄, which may be identical or different, represent an atom or a group chosen from:
hydrogen;
halogen such as bromine and chlorine,
hydroxyl,
C₁-C₄ alkoxy,
hydroxysulfonyl (—SO₃H) or sulfonate (—SO₃⁻, M⁺), with M⁺ representing a cationic counterion;
optionally substituted C₁-C₆ alkyl,
—NR₅R₆ in which R₅ and R₆, which may be identical or different, represent an atom or radical chosen from: i) hydrogen, ii) (C₁-C₄)alkylcarbonyl, iii) arylsulfonyl such as phenylsulfonyl (—SO₂Ph), iv) Het-ALK-C(O)— with Het representing a heterocycloalkyl group which is optionally substituted and ALK represents a (C₁-C₆)alkylene group optionally substituted with one or more hydroxyl or (di)(hydroxy)(C₁-C₄)(alkyl)amino groups; v) optionally substituted aryl, vi) optionally substituted aryl(C₁-C₄)alkyl, viii) optionally substituted C₁-C₂₀ alkyl, optionally interrupted with one or more heteroatoms and/or with one or more groups comprising at least one heteroatom;
a group of formula (a) below:
—N(R₇)—X₁—W₁ (a)
wherein in formula (a):
R₇ represents a hydrogen or a C₁-C₄ alkyl radical,
X₁ represents a divalent radical chosen from C₁-C₂₀ alkylene optionally interrupted with one or more heteroatoms or groups chosen from oxygen, nitrogen, sulfur, CO, SO, SO₂, arylene, or combinations thereof;
W₁ represents a cationic radical chosen from: 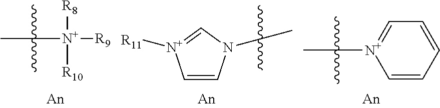
with R₈, R₉, R₁₀ and R₁₁, which may be identical or different, representing a C₁-C₆ alkyl group, a benzyl radical, a C₁-C₆ alkyl sulfonate radical; the radicals R₈ and R₉ optionally form, with the nitrogen atom to which they are attached, a saturated or unsaturated, optionally substituted 5- to 7-membered heterocycle, optionally comprising another non-nitrogen heteroatom;
n is an integer ranging from 1 to 3;
T₁ represents a linear or branched divalent hydrocarbon-based chain comprising from 1 to 20 carbon atoms, optionally interrupted with one or more heteroatoms or groups, or combinations thereof, chosen from oxygen, sulfur, N(R_{b}), C(O), —N⁺(R₈)(R₉)-An, optionally cationic and optionally substituted heteroaryl, An with R₈ and R₉, which may be identical or different, represent a C₁-C₆ alkyl radical; R_{b} representing a hydrogen atom or a (hydroxy)(C₁-C₄)alkyl group; [symbol]
being the part of the bond that is connected to the rest of the molecule; and
(b) at least one cationic (poly)methine fluorescent dye.
